(19) United States
(12) Patent Application Publication
(10) Pub. No.: US 2024/0108633 A1
(43) Pub. Date: Apr. 4, 2024

(54) METHOD FOR PREVENTING OR TREATING DISEASE OR CONDITION ASSOCIATED WITH ANTITUMOR AGENT

(71) Applicant: ONQUALITY PHARMACEUTICALS CHINA LTD., Shanghai (CN)

(72) Inventors: Wenxi LI, Shanghai (CN); Shiyi ZHANG, Shanghai (CN); Qing YOU, Shanghai (CN); Linan YANG, Shanghai (CN); Zhaoyu WU, Shanghai (CN)

(73) Assignee: ONQUALITY PHARMACEUTICALS CHINA LTD., Shanghai (CN)

(21) Appl. No.: 18/259,716

(22) PCT Filed: Dec. 28, 2021
(86) PCT No.: PCT/CN2021/141972

§ 371 Date: Jun. 28, 2023

(30) Foreign Application Priority Data
Dec. 29, 2020 (CN) 202011602295.1

(51) Int. Cl. A61K 31/55; A61K 9/00; A61K 31/437; A61K 31/4985; A61K 31/506; A61K 31/519; A61K 31/5377; A61K 45/06; A61P 17/00

(52) U.S. Cl. CPC A61K 31/55; A61K 9/0014; A61K 31/437; A61K 31/4985; A61K 31/506; A61K 31/519; A61K 31/5377; A61K 45/06; A61P 17/00

(57) ABSTRACT

A JAK inhibitor may be used in preparation of a medicament. The medicament may be used for preventing and/or treating a disease or condition associated with an antitumor agent in a subject. A pharmaceutical composition may include the JAK inhibitor as may a kit. The JAK inhibitor may include a compound of formula (I) or a pharmaceutically acceptable salt thereof:

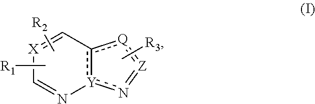

Q, X, Y, and Z being independently N or C, and R₁, R₂ and R₃ being independently a 5-6-membered aromatic ring, 5-6-membered heteroaromatic ring, 5-6-membered cycloalkyl, 5-6-membered heterocycloalkyl, amino, or amido, the aromatic ring, heteroaromatic ring, cycloalkyl, and/or heterocycloalkyl being optionally substituted by a substituent.

### CROSS-REFERENCE TO RELATED APPLICATIONS

This application is a National Stage Application and claims priority under 35 U.S.C. § 371 to Patent Cooperation Treaty application PCT/CN2021/141972, filed Dec. 28, 2021, which claims the benefit of Chinese patent application CN2020116022951 filed Dec. 29, 2020. Priority is claimed to these applications and the disclosures of these prior applications are considered part of the disclosure of this application and to the extent allowed the entire contents of the aforementioned applications are incorporated herein.

### TECHNICAL FIELD

The present application relates to the field of biomedicine, and particularly to a use of a JAK inhibitor in preparing a medicament which is used for preventing or treating diseases or disorders associated with antitumor agents in a subject.

### BACKGROUND

Chemotherapy, radiotherapy and surgery are the most commonly used methods for the treatment of tumors in clinical practice. Chemotherapy is not highly selective, while killing tumor cells, it will cause damage to normal cells with significant side effects. Compared with traditional anti-tumor regimens, targeted therapy targets specific targets on tumor cells (such as a specific gene mutation), immunotherapy uses the body's immune system to attack tumor cells, but because it does not completely distinguish tumor cells and normal cells, or causes abnormal activation of the immune system and still cause side effects, such as, myelosuppression, gastrointestinal toxicity, nephrotoxicity, or hepatotoxicity, thereby adversely affect the efficacy of treatment, and serious adverse events may be life-threatening and shorten the survival of patients.

Mutation or overexpression of epidermal growth factor receptor (EGFR) has been found to be associated with a variety of cancers, and patients suffering from such tumors can be treated by EGFR-inhibiting therapy (e.g., administering EGFR inhibitor). However, this type of therapy will cause severe side effects (especially, in skin, facial organs, and gastrointestinal tract). It has been reported that cutaneous side effects occur in greater than 50% of patients treated with EGFR inhibitors (e.g., see Heidary et al., Journal of the American Academy of Dermatology, 58 (4): 545, 2008), for example, rash. Rash caused by EGFR-inhibiting therapy can result in medicament withdrawal or dose reduction, and can compromise the patient's life quality.

There has been no successful therapeutic regimen in the art for controlling rash caused by EGFR-inhibiting therapy. Thus, there is an urgent need for therapeutic regimens capable of controlling such rashes successfully.

### SUMMARY OF THE INVENTION

In one aspect, the present application provides a use of a JAK inhibitor in preparing a medicament, the medicament is used for preventing or treating diseases or disorders associated with antitumor agents in a subject.

In another aspect, the present application provides a use of a pharmaceutical composition in the preparation of a medicament for preventing or treating a disease or disorder associated with an antitumor agent in a subject, wherein the pharmaceutical composition comprises a JAK inhibitor, and a buffer.

In another aspect, the present application provides a use of a pharmaceutical composition in the preparation of a medicament for preventing or treating a disease or disorder associated with an antitumor agent in a subject, wherein the pharmaceutical composition comprises a JAK inhibitor, and an excipient.

In some embodiments, the JAK inhibitor comprises one or more selected from the following group: a JAK1 inhibitor, a JAK2 inhibitor, a JAK3 inhibitor and a TYK-2 inhibitor.

In some embodiments, the JAK inhibitor comprises an inhibitor that reduces the expression of JAK, and/or an inhibitor that reduces the activity of JAK.

In some embodiments, the JAK inhibitor acts directly on a JAK protein and/or a nucleic acid encoding a JAK protein.

In some embodiments, the JAK inhibitor comprises a small molecular JAK inhibitor, a protein macromolecule that binds specifically to JAK, an RNAi that inhibits expression of a JAK protein and/or an antisense oligonucleotide that inhibits expression of a JAK protein.

In some embodiments, the small molecular JAK inhibitor comprises a small molecular JAK inhibitor that binds reversibly to JAK, a small molecular JAK inhibitor that binds irreversibly to JAK and/or a small molecular JAK inhibitor that binds specifically to mutant JAK.

In some embodiments, the small molecular JAK inhibitor has a molecular weight that is less than or equal to 2000 Daltons, less than or equal to 1500 Daltons, less than or equal to 1200 Daltons, less than or equal to 1000 Daltons, less than or equal to 900 Daltons, less than or equal to 800 Daltons, less than or equal to 700 Daltons, less than or equal to 600 Daltons, less than or equal to 500 Daltons, less than or equal to 400 Daltons, less than or equal to 300 Daltons, less than or equal to 200 Daltons and/or less than or equal to 100 Daltons.

In some embodiments, the JAK inhibitor comprises ruxolitinib, tofacitinib, oclacitinib, fedratinib, peficitinib, upadacitinib, barictinib, fligotinib, delgocitinib, decernotinib, cerdulatinib, lestaurtinib, pacritinib, momelotinib, gandotinib, abrocitinib, solcitinib, SHR-0203, itacitinib, PF-06651600, BMS-986165, cucurbitacin I, CHZ868, TD-1473, zotiraciclib, alkotinib, jaktinib, AZD-4205, DTRMHS-07, KL130008, WXSH-0150, TQ05105, WXFL10203614, GLPG0634, CEP-33779, R₃₄₈, ritlecitinib and/or brepocitinib.

In some embodiments, the JAK inhibitor comprises tasocitinib, deucravacitinib, INCB-039110, izencitinib, entrectinib, ivarmacitinib, deuruxolitinib, adelatinib, NDI-034858, nezulcitinib, ATI-01777, TD-8236, INCB-054707, ropsacitinib, AGA-201, ATI50001, gusacitinib, cerdulatinib, roniciclib, AT-9283, FMX-114, OST-122, TT-00420, repotrectinib, INCB-052793, CT-340, BMS-911543, ilginatinib, BGB-23339, ICP-332, ESK-001, SYHX-1901, VTX-958, TLL-018, CEE-321, CJ-15314, TD-5202, ABBV-712, GLPG-3667, CPL-116, AZD-4604, TAS-8274, MAX-40279, TD-3504, KN-002, AZD-0449, R-548, AC-410, spebrutinib, ONX-0805, AEG-41174, XL-019, CR-4, WP-1066, GDC-0214, INCB-047986, PF-06263276, R-333, AZD-1480, tozasertib, CS-12192, and/or AC-1101.

In some embodiments, the JAK inhibitor comprises peficitinib hydrobromide, fedratinib hydrochloride, tasocitinib citrate, ruxolitinib phosphate, INCB-039110 adipate, momelotinib dihydrochloride, upadacitinib tartrate, jaktinib dihydrochloride monohydrate, ivarmacitinib su lfate, zotiraciclib citrate.

In some embodiments, the JAK inhibitor comprises a compound containing at least one aromatic ring or heteroaromatic ring.

In some embodiments, the JAK inhibitor comprises a compound as shown in Formula I or a pharmaceutically acceptable salt thereof:

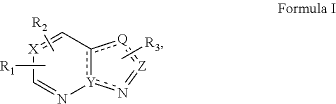

wherein, X is N or C, Y is N or C, Z is N or C, Q is N or C, R₁, R₂ and R₃ are each independently selected from the following group: 5-6-membered aromatic ring, 5-6-membered heteroaromatic ring, 5-6-membered cycloalkyl, 5-6-membered heterocycloalkyl, amino and amido, wherein, the aromatic ring, heteroaromatic ring, cycloalkyl and/or heterocycloalkyl are optionally substituted by a substituent.

In some embodiments, in Formula I, the X is N, the Y is C, the Z is C, and the Q is C.

In some embodiments, in Formula I, the X, Y, Z and Q are all N.

In some embodiments, in Formula I, the X is N, the Y is N, the Z is C, and the Q is C.

In some embodiments, in Formula I, the X is C, the Y is N, the Z is C, and the Q is N.

In some embodiments, in Formula I, the X is C, the Y is C, the Z is N, and the Q is C.

In some embodiments, the R₁ and R₂ are each independently selected from hydrogen atom,

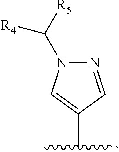

benzene ring, C₁-C₃ alkyl and

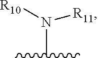

wherein, R₄ is selected from cyclopentyl, cyclobutyl and azetidinyl, the substituent is piperidine, cyanogroup, carbonyl, sulfonyl, the piperidine is further substituted by a substituent, the sulfonyl is further substituted by an alkyl; the R₅ is C₁-C₆ alkyl, the alkyl is further substituted by cyanogroup;

The benzene ring is optionally substituted by acyl, halogen, hydroxyl, C₁-C₃ alkyl, the acyl and alkyl are further optionally substituted by C₃-C₅ cycloalkyl, C₃-C₅ heterocycloalkyl or C₁-C₃ alkyl, the cycloalkyl and heterocycloalkyl are further optionally substituted by C₁-C₃ alkyl;

The R₁₀ and R₁₁ are each independently selected from hydrogen atom, C₁-C₃ alkyl, or 4-10-membered ring, and the ring is a monocyclic ring or a bicyclic ring, the ring is further substituted by amino, sulfonyl, hydroxyl, alkynyl, acyl or C₁-C₃ alkyl, or, the R₁₀ and R₁₁ form a ring.

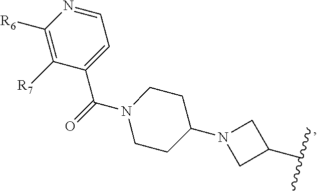

In some embodiments, the R₄ is wherein the R₆ is selected from —CF₃, —CHF₂, —CH₂F and —CH₃, wherein the R₇ is selected from hydrogen atom or fluorine atom.

In some embodiments, the R₄ is selected from cycloalkyl,

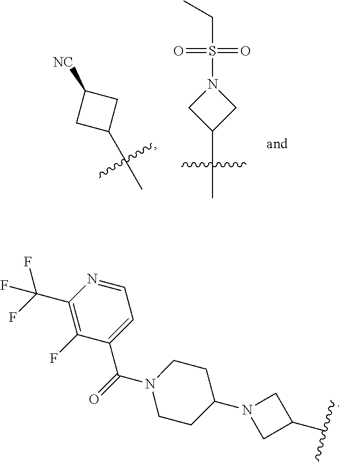

In some embodiments, the R₁ and R₂ are each independently selected from hydrogen atom or benzene ring, the benzene ring is optionally substituted by acyl, halogen, hydroxyl, C₁-C₃ alkyl, the acyl is further optionally substituted by azetidinyl, the azetidinyl is further optionally substituted by methyl.

In some embodiments, the R₁ and R₂ are each independently selected from the following group: hydrogen atom,

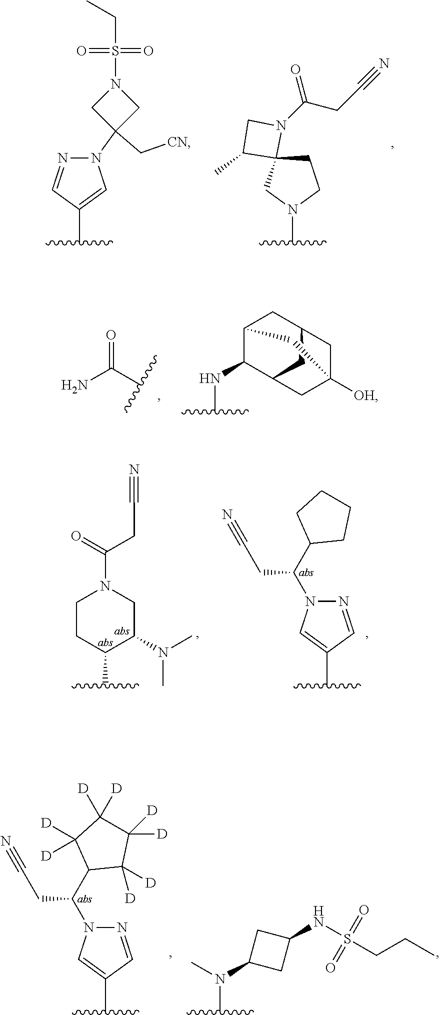

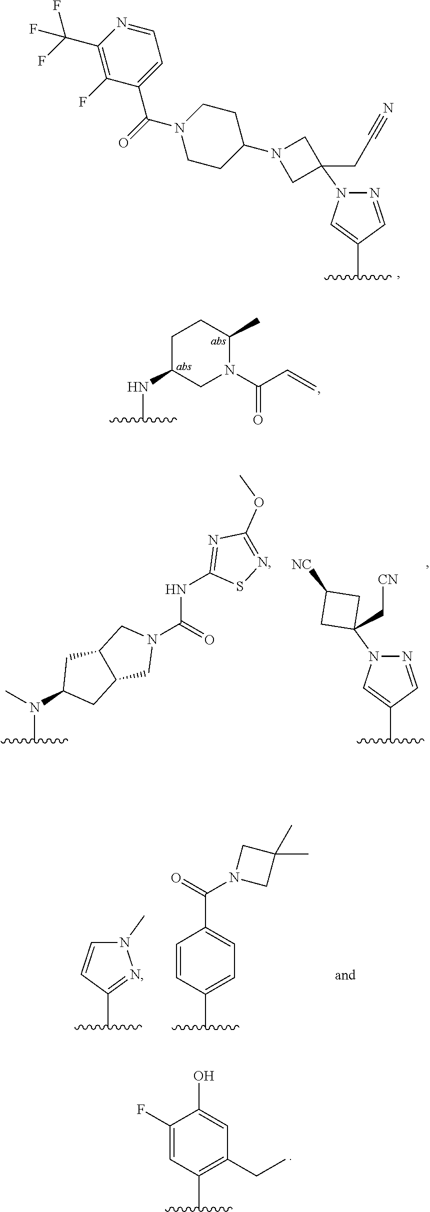

In some embodiments, the R₃ is selected from amido and 5-10 membered aromatic ring, the aromatic ring may be a bicyclic ring and may be substituted by a cyclic group or a chain group.

In some embodiments, the R₃ is amido, the amido is optionally substituted by cyclobutyl or cyclopropyl.

In some embodiments, the R₃ is any one selected from the following group:

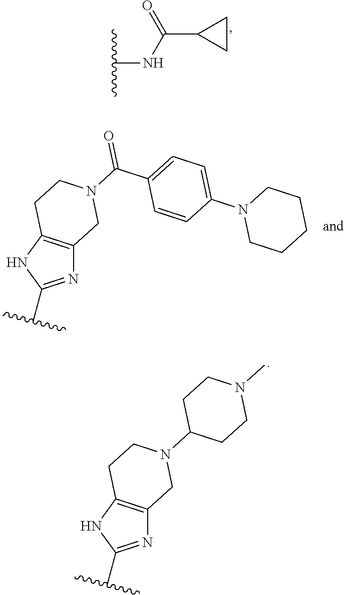 [SMILES: *NC(=O)C1CC1 ; *c1nc2c([nH]1)CCN(C(=O)c1ccc(N3CCCCC3)cc1)C2 ; *c1nc2c([nH]1)CCN(C1CCN(C)CC1)C2]

In some embodiments, JAK inhibitor comprises any one or more of compounds I-1 to I-15:

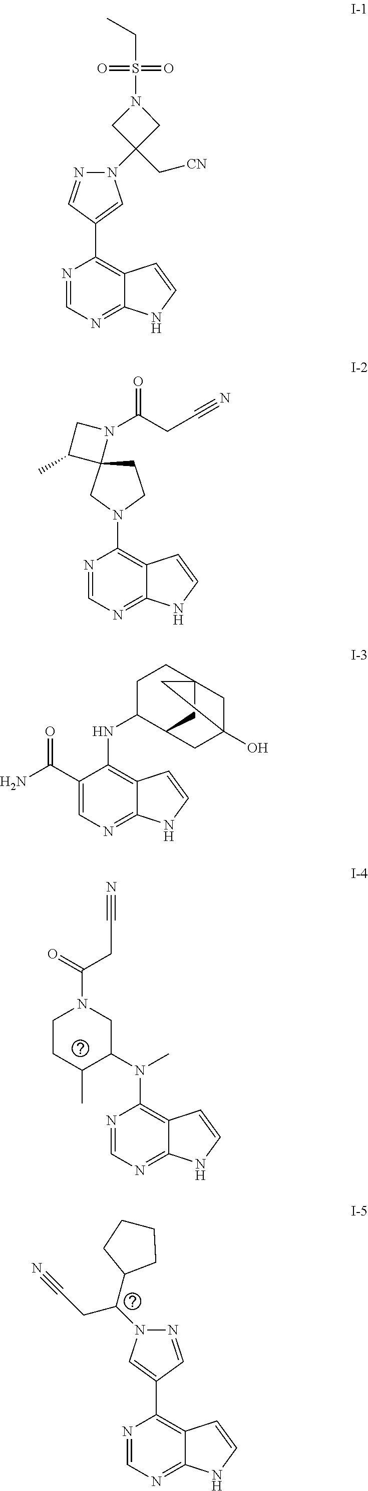 [SMILES: CCS(=O)(=O)N1CC(CC#N)(n2cc(-c3ncnc4[nH]ccc34)cn2)C1 ; C[C@@H]1CN(C(=O)CC#N)[C@@]12CCN(c1ncnc3[nH]ccc13)C2 ; NC(=O)c1cnc2[nH]ccc2c1NC1CCC23C[C@@H]1CC(O)(C2)C3 ; CC1CCN(C(=O)CC#N)CC1N(C)c1ncnc2[nH]ccc12 ; N#CCC(C1CCCC1)n1cc(-c2ncnc3[nH]ccc23)cn1]

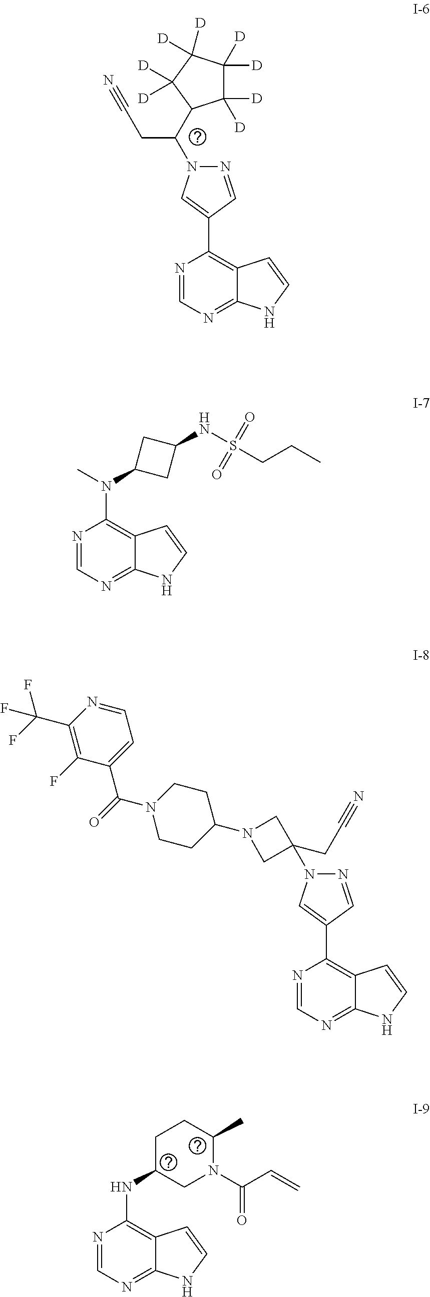 [SMILES: [2H]C1([2H])C(C(CC#N)n2cc(-c3ncnc4[nH]ccc34)cn2)C([2H])([2H])C([2H])([2H])C1([2H])[2H] ; CCCS(=O)(=O)N[C@H]1C[C@@H](N(C)c2ncnc3[nH]ccc23)C1 ; N#CCC1(n2cc(-c3ncnc4[nH]ccc34)cn2)CN(C2CCN(C(=O)c3ccnc(C(F)(F)F)c3F)CC2)C1 ; C=CC(=O)N1C[C@@H](Nc2ncnc3[nH]ccc23)CC[C@H]1C]

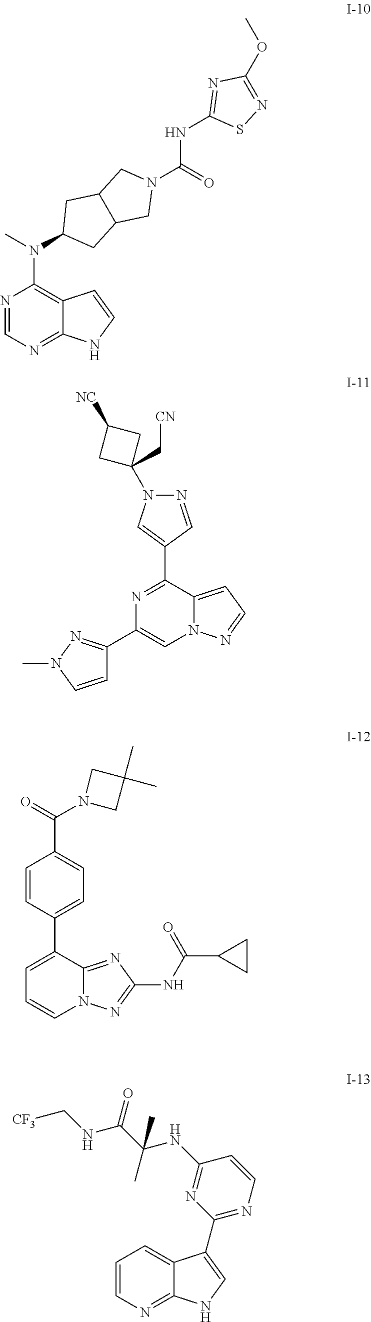

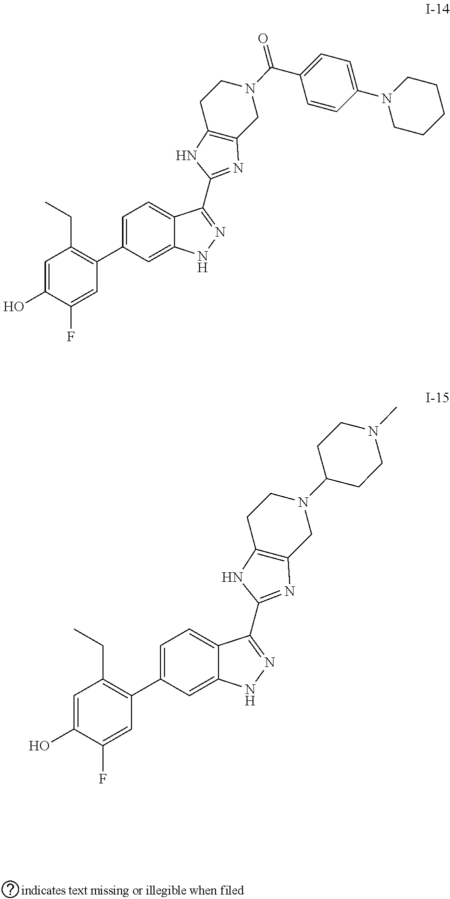 [SMILES: CCc1cc(O)c(F)cc1-c1ccc2c(-c3nc4c([nH]3)CCN(C(=O)c3ccc(N5CCCCC5)cc3)C4)n[nH]c2c1 ; CCc1cc(O)c(F)cc1-c1ccc2c(-c3nc4c([nH]3)CCN(C3CCN(C)CC3)C4)n[nH]c2c1]

In some embodiments, the JAK inhibitor comprises a compound as shown in Formula II:

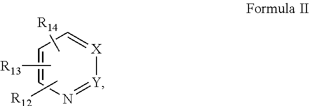

wherein, the X and Y are each independently selected from C or N, and the R₁₂, R₁₃, R₁₄ each independently comprises a group selected from the following group: hydrogen, protium, deuterium, tritium, C₁-C₅ alkyl, halogen, alkoxy, amino, amido, sulfonamido, linear alkyl, cycloalkyl, heterocycloalkyl, alkenyl, alkynyl, aryl and heteroaryl.

In some embodiments, in the compound as shown in Formula II, the X is C, and the Y is N.

In some embodiments, in the compound as shown in Formula II, the X is N, and the Y is C.

In some embodiments, the JAK inhibitor comprises a structure as shown in Formula II-a:

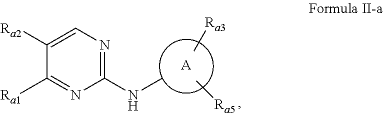

wherein, the Rₐ₁ and Rₐ₂ comprise any substituents as allowed by valance, ring A is aromatic ring or heteroaromatic ring which is optionally substituted by Rₐ₃ and/or Rₐ₅, the Rₐ₃ and Rₐ₅ are each independently selected from: hydrogen atom, cycloalkyl, heterocycloalkyl, aryl, heteroaryl, alkoxy, or, there is a methyl group between the ring A and —NH—.

In some embodiments, the Rₐ₁ is selected from: hydrogen atom, aryl which is optionally substituted by a substituent, heteroaryl which is optionally substituted by a substituent, cycloalkyl which is optionally substituted by a substituent, heterocycloalkyl which is optionally substituted by a substituent, the substituent comprises hydrogen, halogen, alkyl, cyanogroup, sulfonyl, amido.

In some embodiments, the Rₐ₁ is selected from 4-10-membered aromatic cyclyl, 4-10-membered aromatic heterocyclyl, 4-10-membered cycloalkyl, 4-10-membered heterocycloalkyl, the cyclyl is further substituted by amido, the amido is further substituted by cyanogroup, C₁-C₆ alkyl or 5-6-membered heterocyclyl.

In some embodiments, the Rₐ₁ is any one selected from the following group:

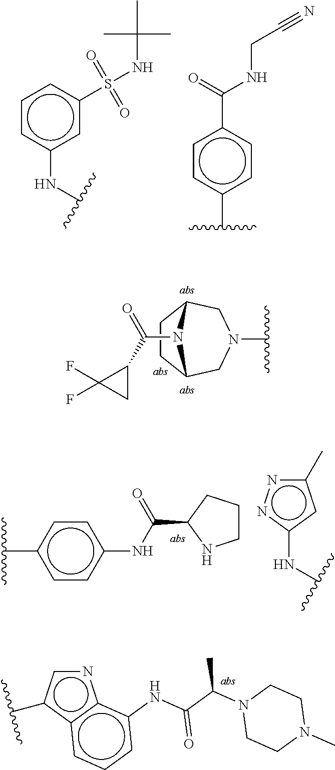

In some embodiments, the Rₐ₂ is selected from: hydrogen, C₁-C₃ alkyl and halogen.

In some embodiments, the Rₐ₂ is selected from: hydrogen, methyl and chlorine.

In some embodiments, the ring A is selected from benzene ring or imidazole ring, the benzene ring or imidazole ring is optionally substituted by C₁-C₃ alkyl, 5-6-membered heterocycloalkyl, 5-6-membered heteroaryl or halogen, the alkyl or ring is further substituted by hydroxyl.

In some embodiments, the Rₐ₁ and Rₐ₅ are each independently selected from the following group: hydrogen atom, methyl, methoxy,

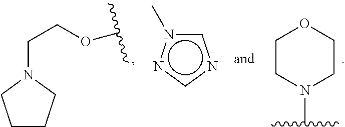

In some embodiments, the R₁₂, R₁₃, R₁₄ are each independently selected from the following group:

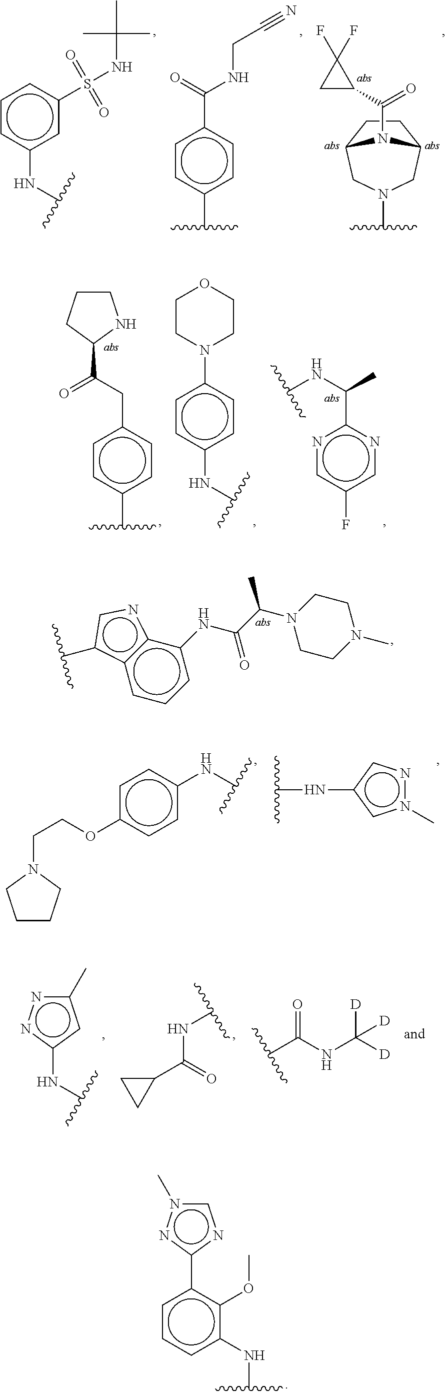

In some embodiments, the JAK inhibitor comprises one or more of compounds II-1 to II-7:

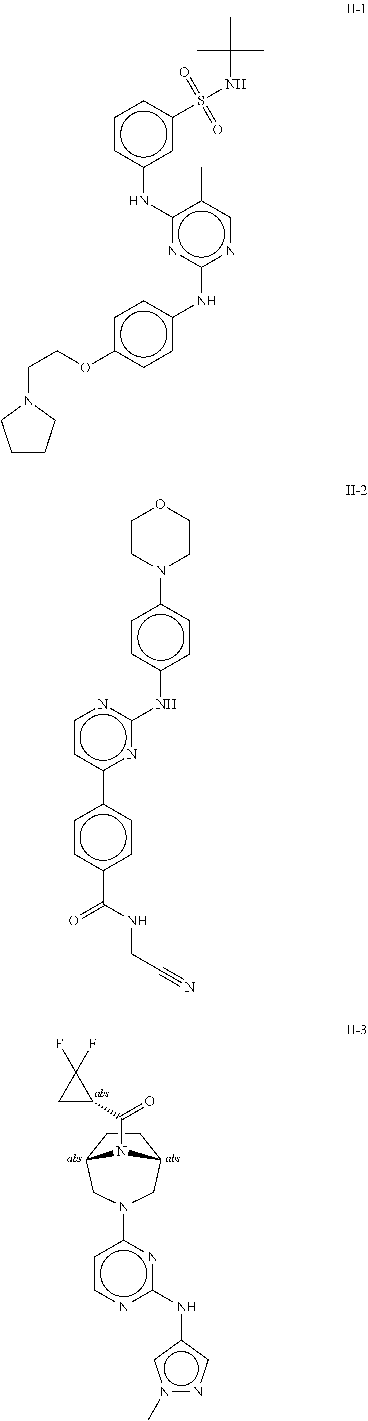

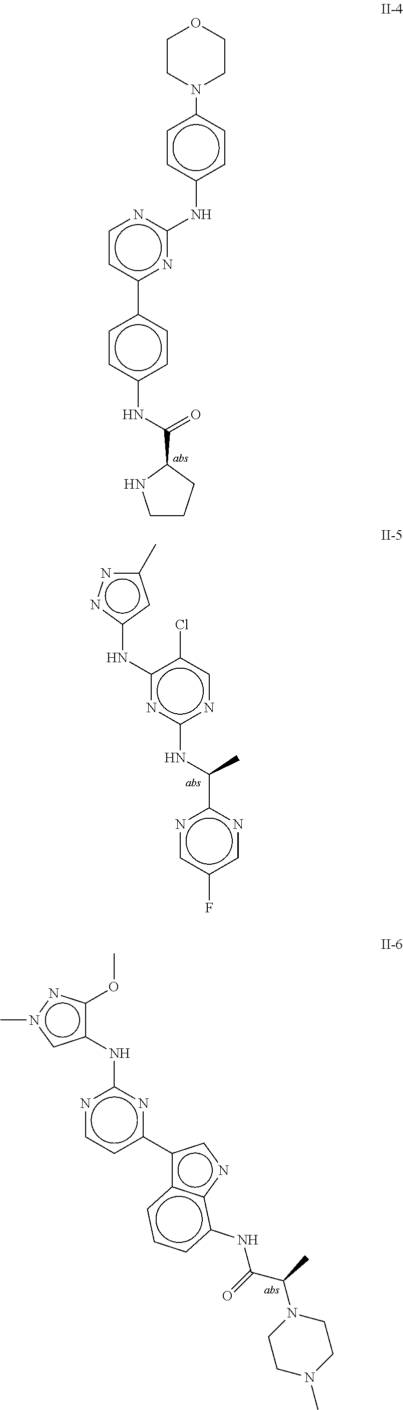

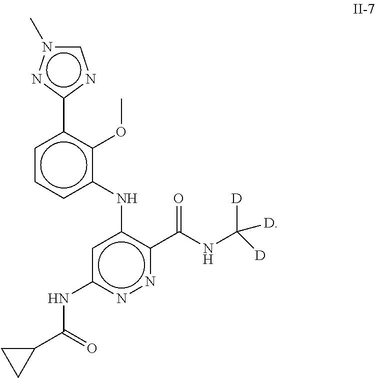

In some embodiments, the JAK inhibitor comprises a compound as shown in Formula III:

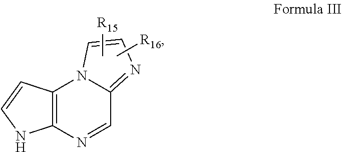

wherein the R₁₅ and R₁₆ are each independently selected from hydrogen atom, cycloalkyl which is optionally substituted by a substituent, heterocycloalkyl which is optionally substituted by a substituent, aryl which is optionally substituted by a substituent and heteroaryl which is optionally substituted by a substituent, the substituent is selected from: amido, alkyl, cycloalkyl, heterocycloalkyl, cyanogroup, amino, hydroxyl and halogen.

In some embodiments, the R₁₅ or the R₁₆ is 4-10-membered heterocycloalkyl, and the heterocycloalkyl is optionally substituted by amido or C₁-C₆ alkyl, the amido is further substituted by C₁-C₆ alkyl, the alkyl is further substituted by halogen.

In some embodiments, the R₁₅ or the R₁₆ is each independently hydrogen atom or

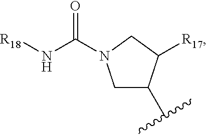

wherein, the R₁₇ and R₁₈ are each independently C₁-C₆ alkyl, and the alkyl is substituted by halogen.

In some embodiments, the R₁₅ and R₁₆ are each independently selected from hydrogen atom and

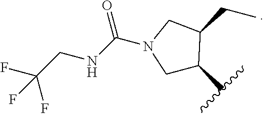 [SMILES: *[C@H]1CN(C(=O)NCC(F)(F)F)C[C@H]1CC]

In some embodiments, the JAK inhibitor comprises a compound III-1:

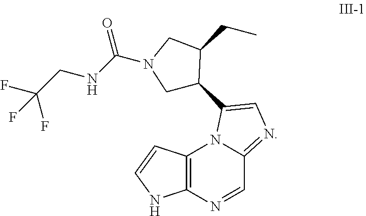 [SMILES: CC[C@@H]1CN(C(=O)NCC(F)(F)F)C[C@@H]1c1cnc2cnc3[nH]ccc3n12]

In some embodiments, the JAK inhibitor comprises a structure as shown in Formula IV:

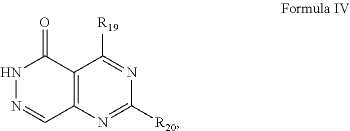

wherein, the R₁₉ and R₂₀ are each independently selected from hydrogen atom, nitro, 4-10-membered cycloalkyl, 4-10-membered heterocycloalkyl, 4-10-membered aryl and 4-10-membered heteroaryl, wherein the nitro, cycloalkyl, heterocycloalkyl, aryl, heteroaryl are further optionally substituted by cyanogroup, alkyl, cycloalkyl, heterocycloalkyl or hydroxyl.

In some embodiments, the R₁₉ is nitro, the nitro is optionally substituted by a substituted benzene ring.

In some embodiments, the substituted benzene ring is substituted by piperidine, the piperidine is further optionally substituted by hydroxyl.

In some embodiments, the R₂₀ is piperidinyl, the piperidinyl is optionally substituted by C₁-C₃ alkyl, the alkyl is further optionally substituted by cyanogroup or hydroxyl.

In some embodiments, the R₂₀ is

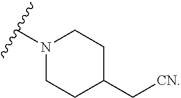 [SMILES: *N1CCC(CC#N)CC1]

In some embodiments, the JAK inhibitor comprises a compound IV-1:

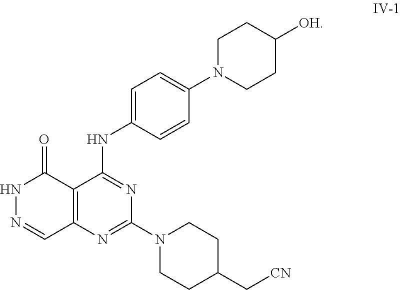 [SMILES: N#CCC1CCN(c2nc(Nc3ccc(N4CCC(O)CC4)cc3)c3c(=O)[nH]ncc3n2)CC1]

In some embodiments, the concentration of the JAK inhibitor in the medicament is 0.01%-10%.

In some embodiments, the antitumor agent comprises a small molecule compound, a small molecule conjugate, a protein and/or a polynucleotide.

In some embodiments, the antitumor agent comprises a targeted therapeutic agent and/or an immunotherapeutic agent.

In some embodiments, the antitumor agent is a targeted therapeutic agent.

In some embodiments, the targeted therapeutic agent comprises a small molecule compound and/or an antibody or an antigen-binding fragment thereof.

In some embodiments, the antibody comprises a monoclonal antibody, a polyspecific antibody, a chimeric antibody, a humanized antibody, a fully human antibody and/or an antibody-drug conjugate.

In some embodiments, the antigen-binding fragment comprises a Fab, Fab′, F(ab)₂, FIT fragment, F(ab′)2, scFv, di-scFv and/or dAb.

In some embodiments, the targeted therapeutic agent targets molecules inside tumor cells, on the cell surface and/or in the tumor microenvironment.

In some embodiments, the targeted therapeutic agent targets proteins and/or nucleic acid molecules of tumor cells.

In some embodiments, the targeted therapeutic agent targets a tumor antigen.

In some embodiments, the targeted therapeutic agent targets EGFR, ALK, MEK, VEGFR, FGFR, PDGFR, ABL, BTK, KIT, AKT, TORC, HER2, HER3, HER4, PI3K, CDK, JAK, ROS1, RET, MET, KRAS, BRAF, BCRP, NTRK, RAS, MSI, PR/ER, BCR/ABL, HDAC, FAK, PYK2, CD20, PD-L1, and/or BRCA1/2, or their mutants.

In some embodiments, the targeted therapeutic agent comprises a hormone therapy, a signal transduction inhibitor, a gene expression modulator, an apoptosis inducer, an angiogenesis inhibitor and and/or a toxin delivery molecule.

In some embodiments, the targeted therapeutic agent is a tyrosine kinase inhibitor.

In some embodiments, the targeted therapeutic agent is an EGFR inhibitor, a MEK inhibitor, an ALK inhibitor, a BTK inhibitor, a PI3K inhibitor, an AKT inhibitor, a VEGFR inhibitor, a mTOR inhibitor, a HDAC inhibitor, a KIT inhibitor, a FGFR inhibitor, a FAK inhibitor, a BCRP inhibitor, an EGFR/cMET inhibitor and/or a SRC inhibitor, and combinations thereof.

In some embodiments, the targeted therapeutic agent is an EGFR inhibitor.

In some embodiments, the targeted therapeutic agent is a VEGFR inhibitor.

In some embodiments, the VEGFR inhibitor is selected from the following group: sulfatinib, anlotinib hydrochloride, tivozanib, lenvatinib, apatinib, intedanib, ponatinib, axitinib, vandetanib, pazopanib hydrochloride and/or sorafenib.

In some embodiments, the targeted therapeutic agent is a FGFR inhibitor.

In some embodiments, the targeted therapeutic agent is a ALK inhibitor.

In some embodiments, the targeted therapeutic agent is a mTOR inhibitor.

In some embodiments, the mTOR inhibitor is selected from the following group: zotarolimus, sirolimus, everolimus and/or temsirolimus.

In some embodiments, the targeted therapeutic agent is a BTK inhibitor.

In some embodiments, the BTK inhibitor is selected from the following group: orelabrutinib, tirabrutinib hydrochloride, zanubrutinib, acalabrutinib, ibrutinib, dasatinib, pirtobrutinib, tolebrutinib, rilzabrutinib, fenebrutinib and/or evobrutinib.

In some embodiments, the targeted therapeutic agent is a MEK inhibitor.

In some embodiments, the MEK inhibitor is selected from the following group: selumetinib sulfate, binimetinib, cobimetinib, trametinib and/or GSK-1120212.

In some embodiments, the targeted therapeutic agent is a PI3K inhibitor.

In some embodiments, the PI3K inhibitor is selected from the following group: umbralisib, alpelisib, duvelisib, copanlisib hydrochloride, idelalisib, zandelisib, buparlisib, enzastaurin hydrochloride, paxalisib, leniolisib, rigosertib, dactolisib, nortriptyline and/or parsaclisib.

In some embodiments, the targeted therapeutic agent is a AKT inhibitor.

In some embodiments, the AKT inhibitor comprises ipatasertib.

In some embodiments, the targeted therapeutic agent is an EGFR/cMET inhibitor.

In some embodiments, the targeted therapeutic agent is a BRAF inhibitor.

In some embodiments, the BRAF inhibitor is selected from the following group: tepotinib, dabrafenib, vemurafenib and/or encorafenib.

In some embodiments, the targeted therapeutic agent comprises a BRAF inhibitor and a MEK inhibitor.

In some embodiments, the targeted therapeutic agent comprises dabrafenib and trametinib.

In some embodiments, the targeted therapeutic agent targeting CD20 is rituximab.

In some embodiments, the antitumor agent is an immunotherapeutic agent.

In some embodiments, the immunotherapeutic agent can alter an immune response in a subject.

In some embodiments, the immunotherapeutic agent can enhance an immune response in a subject.

In some embodiments, the immunotherapeutic agent is an immune checkpoint inhibitor, a modified immune cell and/or a vaccine.

In some embodiments, the immunotherapeutic agent is an antibody.

In some embodiments, the immunotherapeutic agent is a PD-1 inhibitor, a PD-L1 inhibitor and/or a CTLA-4 inhibitor.

In some embodiments, the antitumor agent is selected from the following group: afatinib, dacomitinib, osimertinib, EA1045, gefitinib, almonertinib, pyrotinib, brigatinib, neratinib, olmutinib, bosutinib, icotinib, vandetanib, lapatinib, alflutinib, BPI-7711, mobocertinib, dovitinib, zorifertinib, varlitinib, orelabrutinib, tirabrutinib, zanubrutinib, acalabrutinib, ibrutinib, dasatinib, pirtobrutinib, tolebrutinib, rilzabrutinib, fenebrutinib, evobrutinib, selumetinib, binimetinib, cobimetinib, trametinib, regorafenib, GSK-1120212, alpelisib, duvelisib, copanlisib, idelalisib, nortriptyline, inavolisib, dactolisib, apitolisib, parsaclisib, buparlisib, rigosertib, enzastaurin, paxalisib, leniolisib, ipatasertib, zotarolimus, sirolimus, everolimus, temsirolimus, sorafenib, apatinib, lenvatinib, sunitinib, cabozantinib, axitinib, nintedanib, brivanib, vatalanib, fruquintinib, dabrafenib, vemurafenib, encorafenib, pazopanib, crizotinib, panobinostat, erlotinib, rituximab, panitumumab, cetuximab, ticilimumab, erfonrilimab, BA-3071, MEDI-5752, defactinib, zalifrelimab, cadonilimab, BCD-217, ipilimumab, tremelimumab, quavonlimumab, atezolizumab, durvalumab, camrelizumab, tislelizumab, sintilimab, toripalimab, pembrolizumab, nivolumab, amivantamab, MCLA-129, EMB-01, LY3164530, Roche Glycart anti-EGFR/cMet, Genentech Anti-met/EGFR, Samsung Anti-EGFR/cMet, Merck serono Anti-cMet/EGFR and GB263, and their combinations.

In some embodiments, the disease or disorder comprises a cutaneous disease or disorder and/or a subcutaneous tissue disease or disorder.

In some embodiments, the cutaneous disease or disorder comprises alopecia, body odor, bullous dermatitis, dry skin, eczema, erythema multiforme, erythroderma, lipoatrophy, hair color changes, abnormal hair texture, hirsutism, hyperhidrosis, hyperkeratosis, hypertrichosis, hypohidrosis, hyperlipidemia, nail changes, nail discoloration, nail loss, nail bumps, skin pain, hand-foot syndrome, photosensitivity, pruritus, purpura, acneiform rash, maculopapular rash, scalp pain, skin atrophy, skin hyperpigmentation, skin hypopigmentation, skin induration, skin ulcers, Stevens-Johnson syndrome, subcutaneous emphysema, telangiectasia, toxic epidermal necrosis, rash and/or urticaria.

In some embodiments, the disease or disorder comprises a disease or disorder associated with the combination of two or more antitumor agents.

In some embodiments, the disease or disorder comprises a disease or disorder associated with the antitumor agent in combination with one or more other therapies.

In some embodiments, the disease or disorder comprises a disease or disorder associated with EGFR dysfunction.

In some embodiments, the disease or disorder comprises EGFR dysfunction-associated rash.

In some embodiments, EGFR dysfunction-associated rash comprises EGFR inhibition-associated rash.

In some embodiments, EGFR dysfunction-associated rash comprises immune rash and/or non-immune rash.

In some embodiments, EGFR dysfunction-associated rash comprises EGFR dysfunction-associated acne vulgaris, EGFR dysfunction-associated acne rosacea, EGFR dysfunction-associated pruritus rash, EGFR dysfunction-associated acneiform rash, EGFR dysfunction-associated cellulitis, EGFR dysfunction-associated Lyme disease, EGFR dysfunction-associated allergic reaction, EGFR dysfunction-associated hidradenitis suppurativa, EGFR dysfunction-associated hives, EGFR dysfunction-associated dermatitis, EGFR dysfunction-associated cradle cap, EGFR dysfunction-associated purpura, EGFR dysfunction-associated Pityriasis rosea, EGFR dysfunction-associated erythema, EGFR dysfunction-associated shingles, EGFR dysfunction-associated bruise and/or EGFR dysfunction-associated xanthelasma, EGFR dysfunction-associated melanoma, EGFR dysfunction-associated basal cell carcinoma, EGFR dysfunction-associated squamous cell carcinoma, EGFR dysfunction-associated Kaposi's sarcoma, EGFR dysfunction-associated erythema annulare centrifugum.

In some embodiments, the severity grading of said rash is Grade 1 or above, Grade 2 or above, Grade 3 or above, Grade 4 or above, or Grade 5, as evaluated in accordance with NCI-CTCAE V5.0.

In some embodiments, EGFR inhibition-associated rash comprises EGFR inhibitor administration-associated rash.

In some embodiments, the EGFR inhibitor comprises a medicament for treating a cancer.

In some embodiments, the EGFR inhibitor acts directly on an EGFR protein and/or a nucleic acid encoding an EGFR protein.

In some embodiments, the EGFR inhibitor comprises a small molecular EGFR inhibitor, a protein macromolecule that binds specifically to EGFR, an RNAi that inhibits expression of an EGFR protein and/or antisense oligonucleotide that inhibits expression of an EGFR protein.

In some embodiments, the small molecular EGFR inhibitor comprises a small molecular EGFR inhibitor that binds reversibly to EGFR, a small molecular EGFR inhibitor that binds irreversibly to EGFR and/or a small molecular EGFR inhibitor that binds specifically to mutant EGFR.

In some embodiments, the EGFR inhibitor comprises cetuximab, gefitinib, erlotinib, icotinib, sapitinib, afatinib, lapatinib, vandetanib, neratinib, brigatinib, panitumumab, necitumumab, nimotuzumab, tesevatinib, allitinib, theliatinib, rociletinib, canertinib, AZD3759, YZJ-0318, neptinib, naquotinib, PF-06747775, SPH1188-11, poziotinib, epitinib, varlitinib, alflutinib, HM61713, CK-101, pyrotinib, larotinib, HS-10296, AP32788, simotinib, GMA204, virlitinib, yinlitinib, nazartinib, olmutinib, osimertinib, dacomitinib, abivertinib, EA1045, lazertinib, mobocertinib, savolitinib, almonertinib, trastuzumab, tepotinib, irbinitinib, cemiplimab, pyrotinib, mereletinib, bosutinib, befotertinib, poziotinib, BPI-7711, SKLB-1028, famitinib, dovitinib and/or zorifertinib.

In some embodiments, the EGFR inhibitor is administered in combination with one or more other therapies.

In some embodiments, the subject comprises a cancer patient.

In some embodiments, the subject has been, is being, and/or will be administered with the EGFR inhibitor.

In some embodiments, the medicament does not substantially affect the therapeutic effect of the EGFR inhibitor.

In some embodiments, the medicament is prepared for local administration.

In some embodiments, the site of the local administration is not the occurrence site of cancer or potential metastatic site of cancer.

In some embodiments, the medicament is prepared for topical administration.

In some embodiments, the medicament is prepared for transdermal administration.

In some embodiments, the dosage form of the medicament comprises cream, lotion, gel, ointment, oleamen, spray, liposome preparation, liniment and/or aerosol.

In some embodiments, the medicament further comprises one or more other active ingredients.

In another aspect, the present application provides a use of the JAK inhibitor in preparing a medicament, which is used for preventing or treating disease or disorder associated with antitumor agent.

In another aspect, the present application provides a use of the JAK inhibitor in preparing a medicament, which is used for preventing or treating rash.

In another aspect, the present application provides a method of preventing or treating disease or disorder associated with antitumor agent, comprising administering to a subject in need thereof the JAK inhibitor as described.

In another aspect, the present application provides a method of preventing or treating EGFR dysfunction-associated rash, comprising administering to a subject in need thereof the JAK inhibitor as described.

In some embodiments, the subject has been, is being, and/or will be administered with the EGFR inhibitor.

In another aspect, the present application provides a method of preventing or treating disease or disorder associated with antitumor agent, comprising administering to a subject in need thereof the JAK inhibitor as described in the use.

In another aspect, the present application provides a method of preventing or treating rash, comprising administering to a subject in need thereof the JAK inhibitor as described in the use.

In another aspect, the present application provides a pharmaceutical combination or a kit, comprising: 1) an antitumor agent; and 2) said JAK inhibitor.

In some embodiments, the antitumor agent and the JAK inhibitor are not mixed with each other.

In some embodiments, the antitumor agent and the JAK inhibitor are each independently present in a separate container.

In some embodiments, the JAK inhibitor is prepared for local administration.

In some embodiments, the site of the local administration is not the occurrence site of cancer or potential metastatic site of cancer.

In some embodiments, the JAK inhibitor is prepared for topical administration.

In some embodiments, the JAK inhibitor is prepared for transdermal administration.

In some embodiments, the JAK inhibitor is prepared as cream, lotion, gel, ointment, oleamen, spray, liposome preparation, liniment and/or aerosol.

In some embodiments, the JAK inhibitor in 2) can preventing or treating the disease or disorder associated with administration of the antitumor agent in 1).

In some embodiments, the JAK inhibitor in 2) does not substantially affect the therapeutic effect of the antitumor agent in 1).

In some embodiments, the JAK inhibitor in 2) is administered before, simultaneously with, or after administration of the antitumor agent in 1).

In another aspect, the present application provides a pharmaceutical combination or a kit, comprising: 1) an EGFR inhibitor; and 2) the JAK inhibitor.

In some embodiments, the EGFR inhibitor and the JAK inhibitor are not mixed with each other.

In some embodiments, the EGFR inhibitor and the JAK inhibitor are each independently present in a separate container.

In some embodiments, the JAK inhibitor is prepared for local administration.

In some embodiments, the site of the local administration is not the occurrence site of cancer or potential metastatic site of cancer.

In some embodiments, the JAK inhibitor is prepared for topical administration.

In some embodiments, the JAK inhibitor is prepared for transdermal administration.

In some embodiments, the JAK inhibitor is prepared as cream, lotion, gel, ointment, oleamen, spray, liposome preparation, liniment and/or aerosol.

In some embodiments, the JAK inhibitor in 2) can preventing or treating the disease or disorder associated with administration of the EGFR inhibitor in 1).

In some embodiments, the JAK inhibitor in 2) does not substantially affect the therapeutic effect of the EGFR inhibitor in 1).

In some embodiments, the JAK inhibitor in 2) is administered before, simultaneously with, or after administration of the EGFR inhibitor in 1).

In another aspect, the present application provides a method, comprising the following steps: monitoring diseases or disorders of a subject who has been administered with an antitumor agent; when the monitoring shows that the subject develops diseases or disorders associated with administration of the antitumor agent, administering the JAK inhibitor as described in the use to the subject.

In some embodiments, the method further comprises keep monitoring the diseases or disorders associated with antitumor agent, and optionally reducing or withdrawing the antitumor agent.

In some embodiments, the severity grading of the disease or disorder associated with antitumor agent increases after administration of the antitumor agent.

In some embodiments, the antitumor agent does not comprise the JAK inhibitor.

In some embodiments, cancer is treated by administering the antitumor agent.

In some embodiments, the site of rash is different from the site of cancer.

In some embodiments, the JAK inhibitor is locally administered to the subject.

In some embodiments, the JAK inhibitor is locally administered to a site substantially containing no cancer cells of the subject.

In some embodiments, the JAK inhibitor is administered to a non-cancerous site of the subject.

In another aspect, the present application provides a method, comprising the following steps: monitoring rashes of a subject who has been administered with an EGFR inhibitor; when the monitoring shows that the subject develops rashes associated with administration of the EGFR inhibitor, administering the JAK inhibitor as described in the use to the subject.

In some embodiments, the method further comprises keep monitoring the rashes, and optionally reducing or withdrawing the EGFR inhibitor.

In some embodiments, the severity grading of the rash increases after administration of the EGFR inhibitor.

In some embodiments, before administration of the EGFR inhibitor, the subject does not suffer from the rash.

In some embodiments, the EGFR inhibitor does not comprise the JAK inhibitor.

In some embodiments, cancer is treated by administering the EGFR inhibitor.

In some embodiments, the site of rash is different from the site of cancer.

In some embodiments, the JAK inhibitor is locally administered to the subject.

In some embodiments, the JAK inhibitor is locally administered to a site substantially containing no cancer cells of the subject.

In some embodiments, the JAK inhibitor is administered to a non-cancerous site of the subject.

Other aspects and advantages of the present application will be readily apparent to those skilled in the art from the following detailed description. Only the exemplary embodiments of the present application are shown and described in the following detailed description. As will be appreciated by those skilled in the art, the disclosure of the present application allows persons skilled in the art to modify the disclosed embodiments without departing from the spirit and scope of the invention involved in the present application. Accordingly, the drawings and the description in the specification of the present application are merely exemplary, and not restrictive.

### BRIEF DESCRIPTION OF THE DRAWINGS

Specific features of the invention involved in the present application are set forth in the appended claims. The features and advantages of the invention involved in the present application can be better understood by referring to the exemplary embodiments detailed hereinafter and the accompanying drawings. A brief description of the drawings is as follows:

FIG. 1: shows the photographs of left side, back side, and right side of a rat model where rashes are caused by the EGFR inhibitor of the present application.

FIG. 2: shows the photographs of left side, back side, and right side of typical rats in the control group and the JAK inhibitor group in examples 1 of the present application.

FIG. 3: shows grading results of rashes in the control group and the JAK inhibitor group in examples 1 of the present application.

FIG. 4: shows grading results of rashes in the control group and the JAK inhibitor group in examples 2 of the present application.

FIG. 5: shows the photographs of left side, back side, and right side of typical rats in the control group and the JAK inhibitor group in examples 3 of the present application.

FIG. 6: shows grading results of rashes in the control group and the JAK inhibitor group in examples 3 of the present application.

FIG. 7: shows grading results of rashes in the control group and the JAK inhibitor group in examples 4 of the present application.

FIG. 8: shows the photographs of left side, back side, and right side of typical rats in other dermatologic medicament group and the JAK inhibitor group in examples 5 of the present application.

FIG. 9: shows grading results of rashes in other dermatologic medicament group and the JAK inhibitor group in examples 5 of the present application.

### DETAILED DESCRIPTION

The embodiments of the present invention are described below by way of specific examples, and persons skilled in the art can readily appreciate other advantages and effects of the present invention from the disclosure of the present specification.

### DETAILED DESCRIPTION OF THE INVENTION

Use

The present application provides a use of a JAK inhibitor in the preparation of a medicament for preventing or treating a disease or disorder associated with an antitumor agent in a subject.

In another aspect, the present application also provides a use of a pharmaceutical composition in the preparation of a medicament for preventing or treating a disease or disorder associated with an antitumor agent in a subject, wherein the pharmaceutical composition comprises JAK inhibitor, and buffer.

In another aspect, the present application also provides a use of a pharmaceutical composition in the preparation of a medicament for preventing or treating a disease or disorder associated with an antitumor agent in a subject, wherein the pharmaceutical composition comprises JAK inhibitor, and excipient.

Disease or Disorder Associated with an Antitumor Agent

The present application provides a method for preventing or treating a disease or disorder associated with an antitumor agent. In some embodiments, the disease or disorder associated with an antitumor agent includes side effects associated with an antitumor agent. For example, the disease or disorder associated with the antitumor agent may mean that the disease or disorder is caused by the administration of one or more antitumor agents, and the disease or disorder develops or aggravates after the administration of the antitumor agent.

Where no prophylaxis or treatment is practiced, the disease or disorder will appears or worsens about 1 hour later, about 2 hours later, about 3 hours later, about 4 hours later, about 5 hours later, about 6 hours later, about 7 hours later, about 8 hours later, about 9 hours later, about 10 hours later, about 11 hours later, about 12 hours later, about 1 day later, about 2 days later, about 4 days later, about 7 days later, about 2 weeks later, about 3 weeks later, about 1 month later, about 2 months or more after the administration of the antitumor agent.

For example, the disease or disorder associated with the antitumor agent may include a cutaneous disease or disorder. For example, the disease or disorder associated with the antitumor agent may include subcutaneous tissue disease or disorder. For example, the disease or disorder disease or disorder associated with the antitumor agent may include alopecia, body odor, bullous dermatitis, dry skin, eczema, erythema multiforme, erythroderma, lipoatrophy, hair color changes, abnormal hair texture, hirsutism, hyperhidrosis, hyperkeratosis, hypertrichosis, hypohidrosis, hyperlipidemia, nail changes, nail discoloration, nail loss, nail bumps, skin pain, hand-foot syndrome, photosensitivity, pruritus, purpura, acneiform rash, maculopapular rash, scalp pain, skin atrophy, skin hyperpigmentation, skin hypopigmentation, skin induration, skin ulcers, Stevens-Johnson syndrome, subcutaneous emphysema, telangiectasia, toxic epidermal necrosis, rash and/or urticaria.

For example, the disease or disorder associated with the antitumor agent may be rash.

For example, the disease or disorder disease or disorder associated with the antitumor agent may include alopecia associated with the antitumor agent, body odor associated with the antitumor agent, bullous dermatitis associated with the antitumor agent, dry skin associated with the antitumor agent, eczema associated with the antitumor agent, erythema multiforme associated with the antitumor agent, erythroderma associated with the antitumor agent, lipoatrophy associated with the antitumor agent, hair color changes associated with the antitumor agent, abnormal hair texture associated with the antitumor agent, hirsutism associated with the antitumor agent, hyperhidrosis associated with the antitumor agent, hyperkeratosis associated with the antitumor agent, hypertrichosis associated with the antitumor agent, hypohidrosis associated with the antitumor agent, hyperlipidemia associated with the antitumor agent, nail changes associated with the antitumor agent, nail discoloration associated with the antitumor agent, nail loss associated with the antitumor agent, nail bumps associated with the antitumor agent, skin pain associated with the antitumor agent, hand-foot syndrome associated with the antitumor agent, photosensitivity associated with the antitumor agent, pruritus associated with the antitumor agent, purpura associated with the antitumor agent, acneiform rash associated with the antitumor agent, maculopapular rash associated with the antitumor agent, scalp pain associated with the antitumor agent, skin atrophy associated with the antitumor agent, skin hyperpigmentation associated with the antitumor agent, skin hypopigmentation associated with the antitumor agent, skin induration associated with the antitumor agent, skin ulcers associated with the antitumor agent, Stevens-Johnson syndrome associated with the antitumor agent, subcutaneous emphysema associated with the antitumor agent, telangiectasia associated with the antitumor agent, toxic epidermal necrosis associated with the antitumor agent, rash and/or urticaria associated with the antitumor agent.

For example, the disease or disorder associated with the antitumor agent may include disease or disorder associated with EGFR dysfunction. For example, the disease or disorder associated with EGFR dysfunction may include EGFR dysfunction-associated rash.

In the present application, the antitumor agent may include a small molecule compound, a small molecule conjugate, a protein (such as antibody) and/or a polynucleotide (such as DNA or RNA).

For example, the antitumor agent may be a targeted therapeutic agent.

For example, the targeted therapeutic agent may include small molecule compound. For example, the targeted therapeutic agent may include antibody or fragment of antigen binding thereof. For example, the antibody may include monoclonal antibody, polyspecific antibody, chimeric antibody, humanized antibody, fully human antibody and/or antibody-drug conjugate. For example, the fragment of antigen binding may include Fab, Fab′, F(ab)₂, Fv fragment, F(ab′)2, scFv, di-scFv and/or dAb. For example, the targeted therapeutic agent targets molecules inside tumor cells, on the cell surface and/or in the tumor microenvironment. For example, the targeted therapeutic agent targets proteins and/or nucleic acid molecules of tumor cells. For example, the targeted therapeutic agent targets a tumor antigen. For example, the targeted therapeutic agent targets EGFR, ALK, MEK, VEGFR, FGFR, PDGFR, ABL, BTK, KIT, AKT, TORC, HER2, HER3, HER4, PI3K, CDK, JAK, ROS1, RET, MET, KRAS, BRAF, BCRP, NTRK, RAS, MSI, PR/ER, BCR/ABL, HDAC, FAK, PYK2, CD20, PD-L1, and/or BRCA1/2, or their mutants.

For example, the targeted therapeutic agent may include a hormone therapy, a signal transduction inhibitor, a gene expression modulator, an apoptosis inducer, an angiogenesis inhibitor and and/or a toxin delivery molecule.

For example, the targeted therapeutic agent may be a tyrosine kinase inhibitor. For example, the targeted therapeutic agent may be an EGFR inhibitor, a MEK inhibitor, an ALK inhibitor, a BTK inhibitor, a PI3K inhibitor, an AKT inhibitor, a VEGFR inhibitor, a mTOR inhibitor, a HDAC inhibitor, a KIT inhibitor, a FGFR inhibitor, a FAK inhibitor, a BCRP inhibitor, an EGFR/cMET inhibitor and/or a SRC inhibitor, and combinations thereof.

For example, the targeted therapeutic agent may be an EGFR inhibitor. For example, the EGFR inhibitor include a small molecular EGFR inhibitor, a protein macromolecule that binds specifically to EGFR, an RNAi that inhibits expression of an EGFR protein and/or antisense oligonucleotide that inhibits expression of an EGFR protein. For example, the small molecular EGFR inhibitor include a small molecular EGFR inhibitor that binds reversibly to EGFR, a small molecular EGFR inhibitor that binds irreversibly to EGFR and/or a small molecular EGFR inhibitor that binds specifically to mutant EGFR. For example, the EGFR inhibitor include cetuximab, gefitinib, erlotinib, icotinib, sapitinib, afatinib, lapatinib, vandetanib, neratinib, brigatinib, panitumumab, necitumumab, nimotuzumab, tesevatinib, allitinib, theliatinib, rociletinib, canertinib, AZD3759, YZJ-0318, neptinib, naquotinib, PF-06747775, SPH1188-11, poziotinib, epitinib, varlitinib, alflutinib, HM61713, CK-101, pyrotinib, larotinib, HS-10296, AP32788, simotinib, GMA204, virlitinib, yinlitinib, nazartinib, olmutinib, osimertinib, dacomitinib, abivertinib, EA1045, lazertinib, mobocertinib, savolitinib, almonertinib, trastuzumab, tepotinib, irbinitinib, cemiplimab, pyrotinib, mereletinib, bosutinib, befotertinib, poziotinib, BPI-7711, SKLB-1028, famitinib, dovitinib and/or zorifertinib.

For example, the targeted therapeutic agent can be a VEGFR inhibitor. For example, the VEGFR inhibitor is selected from the following group: sulfatinib, anlotinib hydrochloride, tivozanib, lenvatinib, apatinib, intedanib, ponatinib, axitinib, vandetanib, pazopanib hydrochloride and/or sorafenib.

For example, the targeted therapeutic agent can be a FGFR inhibitor.

For example, the targeted therapeutic agent can be a ALK inhibitor.

For example, the targeted therapeutic agent can be a mTOR inhibitor. For example, the targeted therapeutic agent can be a mTORC inhibitor. For example, the targeted therapeutic agent can be a mTORC1 inhibitor. For example, the targeted therapeutic agent can be a mTORC2 inhibitor. For example, the mTOR inhibitor may be selected from the following group: zotarolimus, sirolimus, everolimus and/or temsirolimus.

For example, the targeted therapeutic agent can be a BTK inhibitor. For example, the BTK inhibitor may be selected from the following group: orelabrutinib, tirabrutinib hydrochloride, zanubrutinib, acalabrutinib, ibrutinib, dasatinib, pirtobrutinib, tolebrutinib, rilzabrutinib, fenebrutinib and/or evobrutinib.

For example, the targeted therapeutic agent can be a MEK inhibitor. For example, the MEK inhibitor may be selected from the following group: selumetinib sulfate, binimetinib, cobimetinib, trametinib and/or GSK-1120212.

For example, the targeted therapeutic agent can be a PI3K inhibitor. For example, the PI3K inhibitor may be selected from the following group: umbralisib, alpelisib, duvelisib, copanlisib hydrochloride, idelalisib, zandelisib, buparlisib, enzastaurin hydrochloride, paxalisib, leniolisib, rigosertib, dactolisib, nortriptyline and/or parsaclisib.

For example, the targeted therapeutic agent can be an AKT inhibitor. For example, the AKT inhibitor can be ipatasertib.

For example, the targeted therapeutic agent can be an EGFR/cMET inhibitor.

For example, the targeted therapeutic agent can be a BRAF inhibitor. For example, the BRAF inhibitor may be selected from the following group: tepotinib, dabrafenib, vemurafenib and/or encorafenib.

For example, the targeted therapeutic agent may include a BRAF inhibitor and a MEK inhibitor. For example, the targeted therapeutic agent may include dabrafenib and trametinib.

For example, the targeted therapeutic agent targeting CD20 can be rituximab.

For example, the immunotherapeutic agent may include an EGFR inhibitor. For example, the immunotherapeutic agent may not include an EGFR inhibitor.

For example, the antitumor agent can be an immunotherapeutic agent. For example, the immunotherapeutic agent can alter an immune response in a subject. For example, the immunotherapeutic agent can enhance an immune response in a subject. For example, the immunotherapeutic agent may be an immune checkpoint inhibitor, a modified immune cell and/or a vaccine. For example, the immunotherapeutic agent can be an antibody. For example, the immunotherapeutic agent may be a PD-1 inhibitor, a PD-L1 inhibitor and/or a CTLA-4 inhibitor.

For example, the immunotherapeutic agent may not include an EGFR inhibitor.

For example, the antitumor agent may be selected from the following group: afatinib, dacomitinib, osimertinib, EA1045, gefitinib, almonertinib, pyrotinib, brigatinib, neratinib, olmutinib, bosutinib, icotinib, vandetanib, lapatinib, alflutinib, BPI-7711, mobocertinib, dovitinib, zorifertinib, varlitinib, orelabrutinib, tirabrutinib, zanubrutinib, acalabrutinib, ibrutinib, dasatinib, pirtobrutinib, tolebrutinib, rilzabrutinib, fenebrutinib, evobrutinib, selumetinib, binimetinib, cobimetinib, trametinib, regorafenib, GSK-1120212, alpelisib, duvelisib, copanlisib, idelalisib, nortriptyline, inavolisib, dactolisib, apitolisib, parsaclisib, buparlisib, rigosertib, enzastaurin, paxalisib, leniolisib, ipatasertib, zotarolimus, sirolimus, everolimus, temsirolimus, sorafenib, apatinib, lenvatinib, sunitinib, cabozantinib, axitinib, nintedanib, brivanib, vatalanib, fruquintinib, dabrafenib, vemurafenib, encorafenib, pazopanib, crizotinib, panobinostat, erlotinib, rituximab, panitumumab, cetuximab, ticilimumab, erfonrilimab, BA-3071, MEDI-5752, defactinib, zalifrelimab, cadonilimab, BCD-217, ipilimumab, tremelimumab, quavonlimumab, atezolizumab, durvalumab, camrelizumab, tislelizumab, sintilimab, toripalimab, pembrolizumab, nivolumab, amivantamab, MCLA-129, EMB-01, LY3164530, Roche Glycart anti-EGFR/cMet, Genentech Anti-met/EGFR, Samsung Anti-EGFR/cMet, Merck serono Anti-cMet/EGFR and GB263, and their combinations.

In the present application, the disease or disorder may comprise a disease or disorder associated with the combination of two or more antitumor agents. In the present application, the disease or disorder may comprise a disease or disorder associated with the antitumor agent in combination with one or more other therapies. In the present application, the disease or disorder may comprise a disease or disorder associated with EGFR dysfunction. In the present application, the disease or disorder may comprise EGFR dysfunction-associated rash.

Rash

The present application provides a method of preventing or treating rash. In the present application, the term “rash” refers to a skin change capable of affecting the color, appearance, or texture of skin. The rash may be localized at only a part of the body, or affect the overall skin. The rash may also comprise urticaria. The rash may be immune rash and/or non-immune rash.

For example, the pathological manifestations of the rash can comprise significant changes in the epidermal growth and/or differentiation of skin, changes in the terminal differentiation of keratinocytes, dense orthokeratosis and epidermal parakeratosis observed in affected and unaffected skin, injuries in sebaceous gland and/or follicular infundibulum, with or without signs of infection, damages in epidermal barrier, epidermal subcorneal laceration, production of cytokines, infiltration of inflammatory cells (e.g., neutrophils, lymphocyte), bacterial infection, capillary telangiectasia, pigmentation and/or dense inflammatory permeability of epithelium.

For example, the clinical manifestations of the rash may be erythema, xeroderma, pruritus, scaly patches, tenderness, burning sensation, cracks, pustule, follicle, ulcer, abscess, red bumps and/or purulent lesions.

For example, the occurrence site of the rash may be epidermis, e.g., including seborrheic areas of skin. For example, the occurrence site of the rash may comprise scalp, face, neck, chest, upper back, limbs, lower back, belly, hip, periodontal area, belly, palms, soles, nails and/or mucosa.

In the present application, the rash may comprise acne vulgaris, papulopustular rash, acne rosacea, pruritus rash (boil), acneiform rash, cellulitis, Lyme disease, allergic reaction, hidradenitis suppurativa, hives, dermatitis, cradle cap, purpura, pityriasis rosea, erythema, shingles, bruise and/or xanthelasma, melanoma, basal cell carcinoma, squamous cell carcinoma, Kaposi's sarcoma, erythema annulare centrifugum, folliculitis, follicular papule, xeroderma, xerotic eczema and/or papillary pustular eruption.

The severity grading of rash may be based on the Common Adverse Event Terminology Criteria (CTCAE) issued by U.S. National Cancer Institute, which is the standard classification and severity grading criteria for adverse events in cancer treatment clinal trials and other oncology settings (NCI-CTCAE V5.0). In some embodiments, the severity grading of epithelial diseases may be Grade 1 or above, Grade 2 or above, Grade 3 or above, Grade 4 or above, or Grade 5, as evaluated in accordance with NCI-CTCAE V5.0.

EGFR Dysfunction

In one aspect, the present application provides a method of preventing or treating EGFR dysfunction-associated rash. In the present application, the term “EGFR” generally refers to Epidermal Growth Factor Receptor, also known as ErbB1 or HER1, that is a 170 kDa transmembrane glycoprotein encoded by c-erbB1 proto-oncogene. EGFR is a member of the human epidermal growth factor receptor (HER) family of receptor tyrosine kinase (RTK), and the family further comprises HER2 (ErbB2), HER3 (ErbB3) and HER4 (ErbB4). EGFR signaling is initiated by ligand binding, and then signal transduction cascade is initiated by inducing conformational changes in the receptor with other ErbB family members, homodimerization or heterodimerization, and trans-autophosphorylation of receptor (see, Ferguson et al., Annu Rev Biophys, 37: 353 to 73, 2008), thereby finally affecting a variety of cell functions (e.g., cell proliferation and survival). The expression of EGFR or the increased kinase activity thereof is associated with a series of human cancers (see, Mendelsohn et al., Oncogene 19: 6550-6565, 2000; GrUnwald et al., J Natl Cancer Inst 95: 851-67, 2003; Mendelsohn et al., Semin Oncol 33: 369-85, 2006). It has been reported that the increased expression of EGFR is found in numerous cancers, such as, glioma, breast cancer, ovarian cancer, cervical cancer, and the like.

In some cases, the EGFR dysfunction-associated rash comprises EGFR inhibition-associated rash. In the present application, the term “EGFR inhibition” comprises the decreased EGFR activity, expression or quantity caused by any reasons (e.g., caused by treatment or the physical conditions of the subject itself). In some embodiments, EGFR inhibition is meant that the EGFR activity or quantity is decreased by at least 10%. In some embodiments, EGFR inhibition is generally meant that the EGFR activity or quantity is decreased by at least 20%, 40%, 50%, 80%, 90%, 95% or more. In some embodiments, the decrease is based on the comparison with the standard value of the same type of subjects (e.g., the same type of healthy persons or the same type of patients). In some embodiments, the decrease is based on the comparison with the value of the same subject earlier.

In some cases, EGFR inhibition is caused by administration of an EGFR inhibitor. In the present application, the term “EGFR inhibitor” generally refers to any EGFR inhibitor that has been known in the art or will be found in the future, including any substance that causes an inhibition of a biological activity associated with the EGFR activity in a subject (including any inhibition of the downstream biological effect caused by the binding of EGFR with its natural ligand(s)) when the substance is administered to the subject. In some embodiments, the EGFR inhibitor comprises any reagent capable of blocking the EGFR activity or any downstream biological effect thereof during its use in the treatment of a cancer.

The EGFR inhibitor may be identified or screened by well-known methods in the art, e.g., by detecting the changes of expression level of EGFR after administering the compound to be tested. The expression level of EGFR may be detected by well-known methods in the art, e.g., immunohistochemistry, PCR, RT-PCR, in situ hybridization, Southern blot, Western blot, Northern blot, spectrophotometry and ELISA, etc.

For example, the EGFR inhibitor is used for treating a cancer in the subject. In the present application, the term “cancer” generally refers to any medical condition, which is mediated by the growth, proliferation, or metastasis of tumors or malignant cells, and causes solid tumors or non-solid tumors (e.g., leukemia).

For example, the EGFR inhibitor may block the kinase activity of an EGFR receptor by directly binding to the intracellular domain of the EGFR receptor; or reduce or block the biological activity of the EGFR receptor by occupying the ligand binding sites or a portion thereof so that the EGFR receptor cannot access its natural ligand; or reduce the EGFR activity by adjusting the dimerization of EGFR polypeptide or adjusting the interaction between the EGFR polypeptide with other proteins to increase the ubiquitination and endocytosis of EGFR.

For example, the EGFR inhibitor may be non-specific EGFR inhibitors, i.e., such inhibitors inhibit other target proteins in addition to EGFR.

For example, the EGFR inhibitor acts directly on an EGFR protein or a nucleic acid encoding an EGFR protein. In some embodiments, the EGFR inhibitor acts directly on an EGFR protein. In the present application, when describing an inhibitor and a target protein, the term “act directly on” means that the inhibitor may directly bind to the target protein without the aid of any other molecules (including covalently binding and non-covalently binding).

For example, the EGFR inhibitor may be a small molecular EGFR inhibitor, a protein macromolecule that binds specifically to EGFR (e.g., an antibody or fragment of antigen-binding thereof) or an RNAi or antisense oligonucleotide that inhibits expression of an EGFR protein. For example, the EGFR inhibitor may be a small molecular EGFR inhibitor or a protein macromolecule that binds specifically to EGFR (e.g., an antibody or fragment of antigen-binding thereof).

In the present application, the term “nucleic acid” generally refers to a polynucleotide molecule consisting of monomeric nucleotides. Nucleic acids comprise ribonucleic acid (RNA), deoxyribonucleic acid (DNA), single-stranded deoxyribonucleic acid (ssDNA), double-stranded deoxyribonucleic acid (dsDNA), short interfering ribonucleic acid (siRNA) and micro-RNA (miRNA). Other non-limiting examples of polynucleotides comprise gene, gene fragment, exon, intron, messenger RNA (mRNA), transfer RNA, ribosome RNA, ribozyme, cDNA, shRNA, single-stranded short or long RNA, recombinant polynucleotide, branched polynucleotide, plasmid, vector, isolated DNA of any sequence, control region, isolated RNA of any sequence, nucleic acid probe and primer. Nucleic acids may be linear or cyclic.

In the present application, the term “RNAi” generally refers to RNA interference technology, which involves a process where exogenous or endogenous double-stranded RNA molecules or small molecular RNAs inhibit the expression or translation of genes by targeting and specifically degrading mRNA.

In the present application, the term “oligonucleotide” generally refers to an oligomer or polymer of ribonucleic acid (RNA) or deoxyribonucleic acid (DNA) or any mimetic or structurally modified nucleic acid thereof. The term comprises naturally oligonucleotides consisting of nucleobases, ribose and covalent nucleoside (backbone), and non-naturally oligonucleotides having similar function.

In the present application, the term “antisense oligonucleotide” generally refers to a single stranded oligocleotide having a nucleobase sequence that may at least partially hybridize with the corresponding region or fragment of the target nucleic acid.

In the present application, the term “small molecular EGFR inhibitor” may comprise a small molecular EGFR inhibitor that binds reversibly to EGFR (e.g., Gefitinib, Erlotinib, Sapitinib and Icotinib), a small molecular EGFR inhibitor that binds irreversibly to EGFR (e.g., Afatinib, Dacomitinib, Lapatinib (e.g., Tykerb®, GW572016 GlaxoSmithKline), Vandetanib (e.g., ZACTIMA™, ZD6474), Lenvatinib, Canertinib, Varlitinib and Neratinib) and/or a small molecular EGFR inhibitor that binds specifically to mutant EGFR (e.g., Osimertinib, Nazartinib, Rociletinib, Olmutinib, Avitinib and EAI045).

The protein macromolecule that binds specifically to EGFR may be an EGFR targeted antibody, an antibody variant, a fusion protein, a derivative or a fragment thereof. In some embodiments, the protein macromolecule that binds specifically to EGFR is an antibody or its fragment of antigen-binding that binds specifically to EGFR.

In the present application, the term “specifically binding”, when used to describe an EGFR inhibitor, generally means that the EGFR inhibitor may recognize EGFR in a complex mixture, and the binding constant of the inhibitor to EGFR is at least 2 times as compared with the binding constant of the inhibitor to other non-specifically binding proteins.

In some cases, the EGFR inhibitor may be administered in combination with one or more other cancer therapies. The other cancer therapies may be conventional means for treating cancers in the art, e.g., cytotoxic anticancer agents, immunotherapy anticancer agents, or hormone therapy anticancer agents. In accordance with the present application, a medicament for treating a cancer may also be used in combination with radiotherapy or surgery therapy. In some embodiments, the EGFR inhibitor and the additional anticancer agents, when used in combination, may be simultaneously administered to a subject, or individually administered at intervals.

EGFR Dysfunction-Associated Rash

The rash of the present application may be EGFR dysfunction-associated rash. In some embodiments, the rash of the present application may be EGFR inhibition-associated rash. In some embodiments, the rash of the present application may be EGFR inhibitor-associated rash. In some embodiments, the rash of the present application may be rash developed after administration of the EGFR inhibitor.

In the present application, the EGFR dysfunction-associated rash may comprise EGFR dysfunction-associated acne vulgaris, EGFR dysfunction-associated papulopustular rash, EGFR dysfunction-associated acne rosacea, EGFR dysfunction-associated pruritus rash, EGFR dysfunction-associated acneiform rash, EGFR dysfunction-associated cellulitis, EGFR dysfunction-associated Lyme disease, EGFR dysfunction-associated allergic reaction, EGFR dysfunction-associated hidradenitis suppurativa, EGFR dysfunction-associated hives, EGFR dysfunction-associated dermatitis, EGFR dysfunction-associated cradle cap, EGFR dysfunction-associated purpura, EGFR dysfunction-associated pityriasis rosea, EGFR dysfunction-associated erythema, EGFR dysfunction-associated shingles, EGFR dysfunction-associated bruise and/or EGFR dysfunction-associated xanthelasma, EGFR dysfunction-associated melanoma, EGFR dysfunction-associated basal cell carcinoma, EGFR dysfunction-associated squamous cell carcinoma, EGFR dysfunction-associated Kaposi's sarcoma, EGFR dysfunction-associated erythema annulare centrifugum, EGFR dysfunction-associated folliculitis, EGFR dysfunction-associated follicular papule, EGFR dysfunction-associated xeroderma, EGFR dysfunction-associated xerotic eczema and/or EGFR dysfunction-associated papillary pustular eruption.

JAK Inhibitor

The present application provides a method of preventing or treating rash, comprising administering a JAK inhibitor.

In the present application, the term “JAK inhibitor” generally refers to a reagent that reduces the expression of Janus kinase 1 (JAK1), Janus kinase 2 (JAK2), Janus kinase 3 (JAK3) or non-receptor protein tyrosine kinase 2 (TYK-2) and/or the kinase activity of at least one of JAK1, JAK2, JAK3 and TYK-2. In some cases, the JAK inhibitor may reduce the expression of JAK1. In some cases, the JAK inhibitor may reduce the expression of JAK2. In some cases, the JAK inhibitor may reduce the expression of JAK3. In some cases, the JAK inhibitor may reduce the expression of TYK-2.

In some cases, the JAK inhibitor may reduce the kinase activity of JAK1. In some cases, the JAK inhibitor may reduce the kinase activity of JAK2. In some cases, the JAK inhibitor may reduce the kinase activity of JAK3. In some cases, the JAK inhibitor may reduce the kinase activity of TYK-2. In some cases, the JAK inhibitor may reduce the kinase activities of JAK1, JAK2, JAK3 and TYK2. In some cases, the JAK inhibitor may reduce the kinase activities of 2 or more (e.g., 3 or 4) of JAK1, JAK2, JAK3 and TYK2. In some cases, the JAK inhibitor may reduce the kinase activity of a single JAK subtype (e.g., JAK1, JAK2, JAK3 or TYK2). In some cases, the JAK inhibitor may reduce the kinase activities of JAK1 and JAK2. In some cases, the JAK inhibitor may reduce the kinase activities of JAK1 and JAK3. In some cases, the JAK inhibitor may reduce the kinase activities of JAK2 and JAK3. In some cases, the JAK inhibitor may reduce the kinase activities of JAK1, JAK2 and JAK3.

In the present application, the JAK inhibitor may comprise inhibitory nucleic acids. In some cases, the JAK inhibitor may comprise antisense nucleotide, ribozyme, small interfering RNA, small hairpin RNA or micro-RNA. In the present application, the inhibitory nucleic acid that can reduce the expression of JAK1, JAK2, JAK3 or TYK2 mRNA in mammalian cells can be synthesized in vitro. These nucleotides can be constructed through chemical synthesis and enzyme-binding reactions using procedures known in the field, and may be modified.

In the present application, the JAK inhibitor may comprise a protein macromolecule that binds specifically to JAK. The protein macromolecule that binds specifically to JAK may be a JAK targeted antibody, an antibody variant, a fusion protein, a derivative or a fragment thereof. In some embodiments, the protein macromolecule that binds specifically to JAK is an antibody or fragment of antigen binding thereof that binds specifically to JAK.

In the present application, the term “specifically binding”, when used to describe a JAK inhibitor, generally means that the JAK inhibitor may recognize JAK in a complex mixture, and the binding constant of the inhibitor to JAK is at least 2 times as compared with the binding constant of the inhibitor to other non-specifically binding proteins.

In the present application, the JAK inhibitor may comprise a small molecular JAK inhibitor. The small molecular JAK inhibitor comprises a small molecular JAK inhibitor that binds reversibly to JAK, a small molecular JAK inhibitor that binds irreversibly to JAK and/or a small molecular JAK inhibitor that binds specifically to mutant JAK.

In some embodiments, the JAK inhibitor may comprise JAK1 and JAK2 inhibitors. For example, the JAK1 and JAK2 inhibitors may comprise, e.g., ruxolitinib (INCB018424), baricitinib (INCB028050 or LY3009104), AZD1480, filgotinib (GLPG0634 or G146034) and/or momelotinib (GS-0387 or CYT387).

In some embodiments, the JAK inhibitor may comprise a JAK1 inhibitor. For example, the JAK1 inhibitor may comprise, e.g., GSK2586184, oclacitinib (PF03394197), upadacitinib, GLG0778, INCB039110, PF04965842 and/or SAR-20347.

In some embodiments, the JAK inhibitor may comprise a JAK2 inhibitor. For example, the JAK2 inhibitor may comprise, e.g., CEP-33779, fedratinib (TG101348, SAR302503), lestaurtinib (CEP-701), pacritinib (SB1518, BMS-911543, XL019, (LY-2784544), R723 and/or Z3.

In some embodiments, the JAK inhibitor may comprise a JAK3 inhibitor. For example, the JAK3 inhibitor may comprise, e.g., decernotinib (VX-509), R348, R256, INCB047986, INCB16562, NVP-BSK805, peficitinib (ASP015K or JNJ-54781532), Tofacitinib (CP-690,500), cucurbitacin I (JSI-124) and/or CHZ868.

In some embodiments, the JAK inhibitor may comprise a TYK2 inhibitor. For example, the TYK2 inhibitor may comprise, e.g., Ndi-031301, BMS-986165, SAR-20347, (4-methoxybenzylidene) malononitrile (tyrphostin A1) and/or triazolopyridine (US 2013/0143915).

In some embodiments, the JAK inhibitor may comprise a pan-JAK inhibitor. In the present application, the term “pan-JAK inhibitor” generally refers to that when IC₅₀ was determined for wild-type human JAK1, wild-type human JAK2, and wild-type human JAK3 using similar test conditions (e.g., the same test conditions), the reagent that has an IC₅₀ of about 500 nM to 4 μM (e.g., about 500 nM to about 2 μM) for each of human JAK1, human JAK2, human JAK3 subtypes. For example, the pan-JAK inhibitor may be a reagent of which the IC₅₀ for wild-type human JAK1, wild-type human JAK2, and wild-type human JAK3 are within ±10% of each other, when each IC₅₀ value is determined under similar test conditions (for example, the same test, e.g., the test for wild-type human JAK1, wild-type human JAK2, and wild-type human JAK3 as described in Kim et, al., J. Med. Chem. 58 (18): 7596-5602, 2015).

In some embodiments, the pan-JAK inhibitor may comprise, e.g., tofacitinib (or CP-690550), cerdulatinib, pyridone 6 (P6), PF-06263276, JAK inhibitor 1 (CAS 457081-03-07) and/or baricitinib.

In some embodiments, the JAK inhibitor may comprise selective JAK1/JAK3 inhibitors. In the present application, the term “selective JAK1/JAK3 inhibitors” generally refers to the reagents of which the IC₅₀ for wild-type human JAK1 and wild-type JAK3 are at least 5 times (e.g., at least 10 times, or at least 20 times) lower than the IC₅₀ for wild-type human JAK2, when IC₅₀ is determined for each of wild-type human JAK1, wild-type human JAK2 and wild-type human JAK3 using similar test conditions (e.g., the same test, for example, the test for wild-type human JAK1, wild-type human JAK2, and wild-type human JAK3 as described in Kim et, al., J. Med. Chem. 58 (18): 7596-5602, 2015).

In some embodiments, the JAK inhibitor may comprise a selective JAK1 inhibitor. In the present application, the term “selective JAK1 inhibitor” generally refers to the reagent of which the IC₅₀ for wild-type human JAK1 is at least 10 times (e.g., at least 20 times) lower than each of the IC₅₀ for wild-type human JAK2 and the IC₅₀ for wild-type human JAK3, when IC₅₀ is determined using similar test conditions (e.g., the same test, for example, the test for wild-type human JAK1, wild-type human JAK2, and wild-type human JAK3 as described in Kim et, al., J. Med. Chem. 58 (18): 7596-5602, 2015).

In some embodiments, the JAK inhibitor may comprise a selective JAK2 inhibitor. In the present application, the term “selective JAK2 inhibitor” generally refers to the reagent of which the IC₅₀ for wild-type human JAK2 is at least 10 times (e.g., at least 20 times) lower than each of the IC₅₀ for wild-type human JAK1 and the IC₅₀ for wild-type human JAK3, when IC₅₀ is determined using similar test conditions (e.g., the same test, for example, the test for wild-type human JAK1, wild-type human JAK2, and wild-type human JAK3 as described in Kim et, al., J. Med. Chem. 58 (18): 7596-5602, 2015).

In the present application, the JAK inhibitor may have a molecular weight less than or equal to 2000 Daltons, less than or equal to 1500 Daltons, less than or equal to 1200 Daltons, less than or equal to 1000 Daltons, less than or equal to 900 Daltons, less than or equal to 800 Daltons, less than or equal to 700 Daltons, less than or equal to 600 Daltons, less than or equal to 500 Daltons, less than or equal to 400 Daltons, less than or equal to 300 Daltons, less than or equal to 200 Daltons and/or less than or equal to 100 Daltons.

In the present application, the JAK inhibitor may comprise ruxolitinib, tofacitinib, oclacitinib, fedratinib, peficitinib, upadacitinib, barictinib, fligotinib, decernotinib, cerdulatinib, lestaurtinib, pacritinib, momelotinib, gandotinib, abrocitinib, solcitinib, SHR-0203, itacitinib, PF-06651600, BMS-986165, abrocitinib, Cucurbitacin I, CHZ868, TD-1473, zotiraciclib, alkotinib, jaktinib, AZD-4205, DTRMHS-07, KL130008, WXSH-0150, TQ05105, WXFL10203614, GLPG0634, CEP-33779, R₃₄₈, ritlecitinib, brepocitinib, tasocitinib, deucravacitinib, INCB-039110, izencitinib, entrectinib, ilvarmacitinib, deuruxolitinib, adelatinib, NDI-034858, nezulcitinib, ATI-01777, TD-8236, INCB-054707, ropsacitinib, AGA-201, ATI50001, gusacitinib, cerdulatinib, roniciclib, AT-9283, FMX-114, OST-122, TT-00420, repotrectinib, INCB-052793, CT-340, BMS-911543, ilginatinib, BGB-23339, ICP-332, ESK-001, SYHX-1901, VTX-958, TLL-018, CEE-321, CJ-15314, TD-5202, ABBV-712, GLPG-3667, CPL-116, AZD-4604, TAS-8274, MAX-40279, TD-3504, KN-002, AZD-0449, R-548, AC-410, spebrutinib, ONX-0805, AEG-41174, XL-019, CR-4, WP-1066, GDC-0214, INCB-047986, PF-06263276, R-333, AZD-1480, tozasertib, CS-12192, and/or AC-1101.

In the present application, the JAK inhibitor may comprise peficitinib hydrobromide, fedratinib hydrochloride, tasocitinib citrate, ruxolitinib phosphate, INCB-039110 adipate, momelotinib dihydrochloride, upadacitinib tartrate, jaktinib dihydrochloride monohydrate, ivarmacitinib sulfate and/or zotiraciclib citrate.

Compounds as Shown in Formula I, Formula II and Formula III

In the present application, the term “alkyl” generally refers to a linear or branched saturated hydrocarbon substituent (e.g., a substituent obtained by dehydrogen from a hydrocarbon) comprising 1-20 carbon atoms; e.g., 1-12 carbon atoms; in other embodiments, the carbon atom number is 1-10; in other embodiments, 1-6 carbon atoms; in other embodiments, 1-4 carbon atoms (e.g., 1, 2, 3 or more carbon atoms). Examples of substituents comprise, e.g., methyl, ethyl, propyl (including n-propyl and iso-propyl), butyl (including n-butyl, iso-butyl, sec-butyl, and tert-butyl), pentyl, iso-pentyl, hexyl, etc. In some cases, the carbon atom number in a hydrocarbon substituent (i.e., alkyl, alkenyl, cycloalkyl, aryl, etc.) is represented by a prefix “Cₐ-C_{b}”, wherein “a” is the lower limit of the carbon atom number, and “b” is the upper limit of the carbon atom number. Therefore, for example, “C₁-C₆ alkyl” refers to an alkyl substituent group comprising 1 to 6 carbon atoms, including linear or branched methyl, ethyl, propyl, butyl, pentyl and hexyl.

In the present application, the term “cycloalkyl” generally refers to a carbocyclyl substituent obtained by dehydrogen from a saturated carbocyclyl ring molecule and having 3-14 carbon atoms. In some embodiments, a cycloalkyl substituent has 3-10 carbon atoms. The cycloalkyl group may be a monocycle that generally comprises 4-7 ring atoms. The cycloalkyl groups comprise cyclopropyl, cyclobutyl, cyclopentyl and cyclohexyl. The cycloalkyl group can also be 2-3 rings fused to each other, which can also be called “bicycloalkyl”. In the present application, the term “cycloalkyl” further comprises a substituent fused to C₆-C₁₀ aromatic ring or 5-10-membered heteroaromatic ring, wherein a group having such a fused cycloalkyl as a substituent bind to the carbon atom in the cycloalkyl. When the fused cycloalkyl group is substituted with one or more substituents, unless otherwise indicated, the one or more substituents are each bonded to a carbon atom in the cycloalkyl. The fused C₆-C₁₀ aromatic ring or 5-10-membered heteroaromatic ring can optionally be further substituted.

In the present application, the term “alkenyl” generally refers to a linear or branched aliphatic hydrocarbyl containing at least one carbon-carbon double bond. “C₁-C₆ alkenyl” refers to alkenyl substituents containing 1 to 6 carbon atoms, including linear or branched vinyl, propenyl, butenyl, pentenyl and hexenyl. In the present application, the term “alkynyl” generally refers to a linear or branched aliphatic hydrocarbyl containing at least one carbon-carbon triple bond. “C₁-C₆ alkynyl” refers to alkynyl substituents containing 1 to 6 carbon atoms, including linear or branched ethynyl, propynyl, butynyl, pentynyl and hexynyl.

In the present application, the term “deuterium” generally refers to a stable form isotope of hydrogen, also known as heavy hydrogen, and commonly indicated with an elemental symbol D or 2H. Its atomic nucleus is consisted of one proton and one neutron. In the present application, the term “hydroxyl” generally refers to a group with a chemical formula of —OH. In the present application, the term “amino” generally refers to a group with a chemical formula of —NH₂. In the present application, the term “cyanogroup” generally refers to a group with a chemical formula of —CN. In the present application, the term “nitro” generally refers to a group obtained by removing one hydroxyl from a nitric acid molecule. In the present application, the term “halogen” generally comprises fluorine, chlorine, bromine and iodine. In the present application, the term “hydrogen” generally refers to a hydrogen substituent, which may be described as —H. In the present application, the term “oxygen” generally refers to an oxygen substituent, which may be described as —O—.

In the present application, the terms “substituents”, “radicals” and “groups” can be used interchangeably.

If a substituent is described as “optionally substituted”, the substituent may be: (1) unsubstituted, or (2) substituted. If a carbon of a substituent is described as optionally substituted with one or more substituents, one or more hydrogens on the carbon (if present) may be substituted with independently selected optional substituents, alone or together. If nitrogen of a substituent is described as optionally substituted with one or more substituents, one or more hydrogens on the nitrogen (if present) may be each substituted with independently selected optional substituents. An exemplary substituent may be described as —NR′R″, wherein R′ and R″, together with the nitrogen atom to which they are attached, can form a heterocyclyl ring comprising one or two heteroatoms independently selected from the group consisting of oxygen, nitrogen, and sulfur, wherein the heterocycloalkyl moiety may be optionally substituted. The heterocyclyl ring formed by R′ and R″, together with the nitrogen atom to which they are attached may be partially or completely saturated, or aromatic.

In the present application, the term “Formula I (or Formula II, Formula III)” may be called as “the compound of Formula I (or Formula II, Formula III), “the compound as shown in Formula I (or Formula II, Formula III)”. Such a term is also defined as comprising all the forms of the compound of Formula I (or Formula II, Formula III), including hydrate, solvate, isomer, crystalline and non-crystalline form, isomorphism, polymorphism, and metabolite. For example, the compound of Formula I (or Formula II, Formula III) or a pharmaceutically acceptable salt thereof may be present in an unsolvated or solvated form. When the binding force of solvent or water thereto is relatively strong, the coordination compound has a definite stoichiometry that is not affected by humidity. However, if the binding force of solvent or water thereto is relatively weak, e. g., in a channel solvate and hygroscopic compound, the content of water/solvent will depend on the humidity and drying conditions. In this case, non-stoichiometry is normal.

The “compound of Formula I (or Formula II, Formula III)” may comprise chiral carbon atom(s). In the present application, the carbon-carbon bonds in the compound of Formula I (or Formula II, Formula III) may be represented by solid lines, solid wedges, or dot wedges. Depicting the bond to a chiral carbon atom with solid lines means that all the potential stereoisomers (e. g., specific enantiomers, racemates, etc.) on the carbon atom are comprised. The compound of the present application may comprise more than one chiral carbon atoms. In these compounds, depicting the bond to a chiral carbon atom with solid lines is intended to indicate that all the potential stereoisomers should be encompassed. For example, unless otherwise indicated, the compound of Formula I (or Formula II, Formula III) may be present as enantiomers, diastereomers, or racemates and mixtures thereof. Depicting the bond to one or more chiral carbon atoms in the compound of Formula I (or Formula II, Formula III) with solid lines and depicting the bond to another chiral carbon atom in the same compound with solid or dot wedges indicate the presence of a mixture of diastereomers.

The compound of the present application may be present as inclusion compounds or other coordination compounds. The present invention encompasses complexes, e.g., inclusion compounds, drug-host inclusion complex, wherein in contrast to the aforesaid solvates, drug and host are present in stoichiometric or non-stoichiometric amount. The present invention further comprises a coordination compound of Formula I (or Formula II, Formula III) comprising two or more organic and/or inorganic components that may be stoichiometric or non-stoichiometric. The resultant complex may be ionized, partially ionized, or unionized.

The stereoisomer of Formula I (or Formula II, Formula III) comprises cis- and trans-isomers, optical isomers, e. g., R and S enantiomers, diastereomers, geometrical isomers, rotamers, conformational isomers and tautomers, the compound of Formula I (or Formula II, Formula III), including the compounds exhibiting one or more of the aforesaid types of isomerisms, and mixtures thereof (e.g., racemate and diastereomer pair). The stereoisomer further comprises acid or base addition salts in which the counter ion has optical activity, e.g., D-lactate or L-lysine, or racemates, e.g., DL-tartrate or DL-arginine.

When any racemate crystallizes, there may be two different types of crystals. The first type is the racemic compounds (true racemates) as described above, wherein a homogeneous form of crystal is produced and comprises two enantiomers in equimolar amounts. The second type is a racemic mixture or agglomerate, wherein two forms of crystals are produced in equimolar amounts, each comprising a single enantiomer.

The compound of Formula I (or Formula II, Formula III) can exhibit tautomerism and structural isomerism. For example, the compound of Formula I (or Formula II, Formula III) may be present in several tautomeric forms, comprising enol and imine forms, and ketone and enamine forms; as well as geometric isomers and their mixtures. All these tautomeric forms are encompassed within the scope of the compound of Formula I (or Formula II, Formula III). Tautomers are present in solution as a mixture of tautomers. In the solid form, one tautomer generally dominates. Even if one tautomer may be described, the present invention also comprises all the tautomers of the compound of Formula I (or Formula II, Formula III).

The present invention further comprises isotope-labeled compounds that are the same as that of Formula I (or Formula II, Formula III) except that one or more atoms thereof are replaced with atoms having atomic mass or mass number different from those found in nature. Isotopes which may be incorporated into the compound of Formula I (or Formula II, Formula III) comprise isotopes of hydrogen, carbon, nitrogen, oxygen, phosphorus, fluorine and chlorine, e. g., but are not limited to: ²H, ³H, ¹³C, ¹⁴C, ¹⁵N, ¹⁸O, ¹⁷O, ³¹P, ³²P, ³⁵S, ¹⁸F, and ³⁶Cl. Some isotope-labeled compounds of Formula I (or Formula II, Formula III), e. g., into which radioisotopes (e. g., ³H and ¹⁴C) are incorporated, may be used for measuring the distribution of drugs and/or substrate tissues due to their easy preparation and detectability. Heavier isotopes, such as, 2H, can provide certain therapeutic advantages due to its greater metabolic stability, e. g., longer half-life in the body or lower dose requirements. The isotope-labeled compound of Formula I (or Formula II, Formula III) generally may be prepared with isotope-labeled reagents instead of non-isotope-labeled reagents.

The compound of the present application may be used as salts derived from inorganic or organic acids. Some compounds have advantages, such as, enhanced drug stability at different temperatures and humidities, or desired solubilities in water/oil due to the physical properties of one or more salts. In some cases, the salts of the compound can also be adjuvants for use in the separation, purification, and/or resolution of the compound.

In the present application, for the group in the chemical formula, wherein [symbol] represents the linking site.

In the present application, the JAK inhibitor comprises a compound as shown in Formula I or a pharmaceutically acceptable salt thereof:

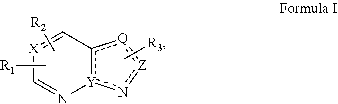

wherein, X may be N or C, Y may be N or C, Z may be N or C, and R₁, R₂ and R₃ may be each independently selected from the following group: 5-6-membered aromatic ring, 5-6-membered heteroaromatic ring, 5-6-membered cycloalkyl, 5-6-membered heterocycloalkyl, amino and amido, wherein, the aromatic ring, heteroaromatic ring, cycloalkyl and/or heterocycloalkyl are optionally substituted by a substituent.

In the present application, in the compound as shown in Formula I, the X may be N, the Y may be C, the Z may be C, and the Q may be C.

In the present application, in the compound as shown in Formula I, the X, Y, Z and Q may all be N.

In the present application, in the compound as shown in Formula I, the X may be N, the Y may be N, the Z may be C, and the Q may be C.

In the present application, in the compound as shown in Formula I, the X may be C, the Y may be N, the Z may be C, and the Q may be N.

In the present application, in the compound as shown in Formula I, the X may be C, the Y may be C, the Z may be N, and the Q may be C.

In the present application, in the compound as shown in Formula I, R₁ and R₂ may be each independently hydrogen atom or

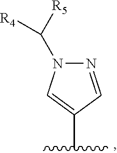

wherein, R₄ may be selected from cyclopentyl, cyclobutyl and azetidinyl, the substituent is piperidine, cyanogroup, carbonyl, sulfonyl, the piperidine may be further substituted by a substituent, the sulfonyl is further substituted by an alkyl.

For example, the R₄ may be

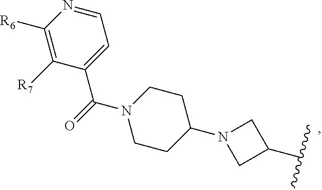

wherein the R₆ may be selected from —CF₃, —CHF₂, —CH₂F and —CH₃, wherein the R₇ may be selected from hydrogen atom or fluorine atom.

For example, the R₄ may be selected from cycloalkyl, cyano-substituted cyclobutyl, sulfonyl-substituted azetidinyl and

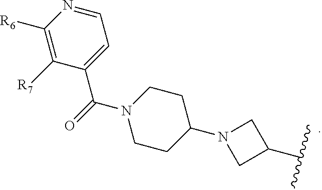

For example, the R₄ may be selected from cycloalkyl,

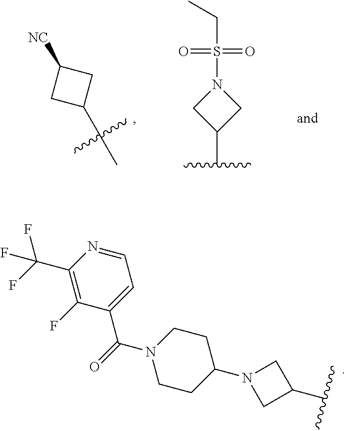

In

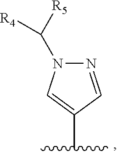

### the R₅ may be —C—CN.

In the present application, the JAK inhibitor may comprise the compound as shown in Formula I, wherein, the R₁ and R₂ may be each independently selected from hydrogen atom,

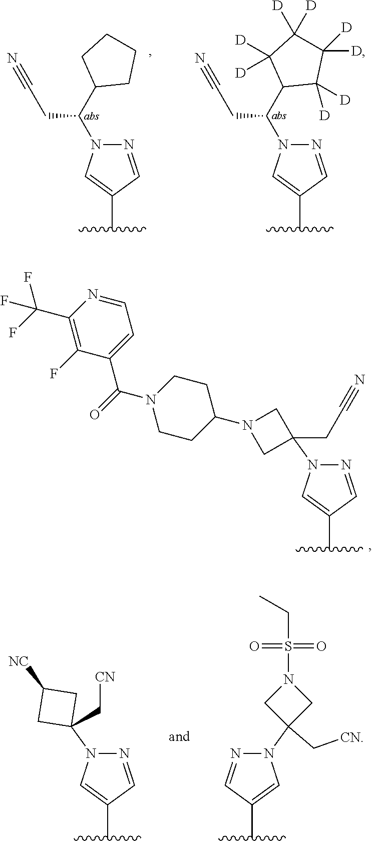

In the present application, the R₁ and R₂ may be each independently selected from hydrogen atom or benzene ring, the benzene ring may be optionally substituted by acyl, halogen, hydroxyl, C₁-C₃ alkyl, the acyl and alkyl may be further optionally substituted by C₃-C₅ cycloalkyl, C₃-C₅ heterocycloalkyl or C₁-C₃ alkyl, the cycloalkyl, heterocycloalkyl may be further optionally substituted by C₁-C₃ alkyl.

For example, R₁ and R₂ may be each independently selected from hydrogen atom or benzene ring, the benzene ring may be optionally substituted by acyl, halogen, hydroxyl, C₁-C₃ alkyl, the acyl may be further optionally substituted by azetidinyl, the azetidinyl may be further optionally substituted by methyl.

For example, R₁ and R₂ may be each independently selected from hydrogen atom,

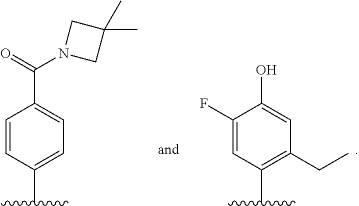 [SMILES: *c1ccc(C(=O)N2CC(C)(C)C2)cc1 ; *c1cc(F)c(O)cc1CC]

In the present application, the R₁ and R₂ may be each independently selected from hydrogen atom, C₁-C₃ alkyl and

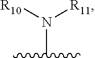

wherein the R₁₀ and Ru may be each independently selected from hydrogen atom, C₁-C₃ alkyl, or 4-10-membered ring, the ring may be optionally substituted by a substituent, and the ring may be a monocyclic ring or a bicyclic ring, the ring may be further substituted by amino, sulfonyl, hydroxyl, alkynyl, acyl or C₁-C₃ alkyl.

Wherein, the R₁₀ and R₁₁ may form a ring.

For example, the R₁ and R₂ may be each independently selected from hydrogen atom, methyl,

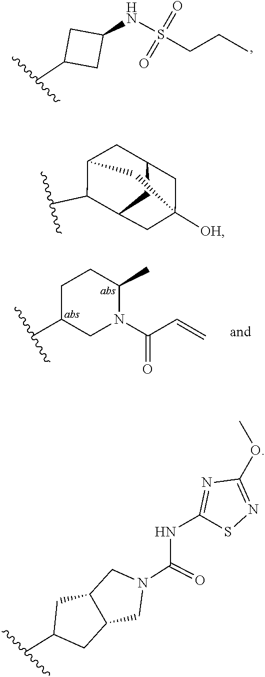

In the present application, the R₁ may be

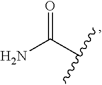 [SMILES: *C(N)=O]

### and the R₂ may be

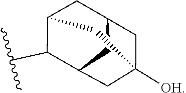 [SMILES: *C1[C@@H]2C[C@@H]3C[C@H]1C[C@@](O)(C3)C2]

In the present application, the R₁ may be

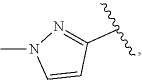 [SMILES: *c1ccn(C)n1]

### and the R₂ may be

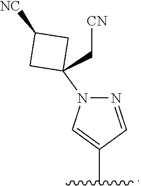 [SMILES: *c1cnn([C@]2(CC#N)C[C@@H](C#N)C2)c1]

In the present application, the R₁ may be hydrogen atom, and the R₂ may be selected from

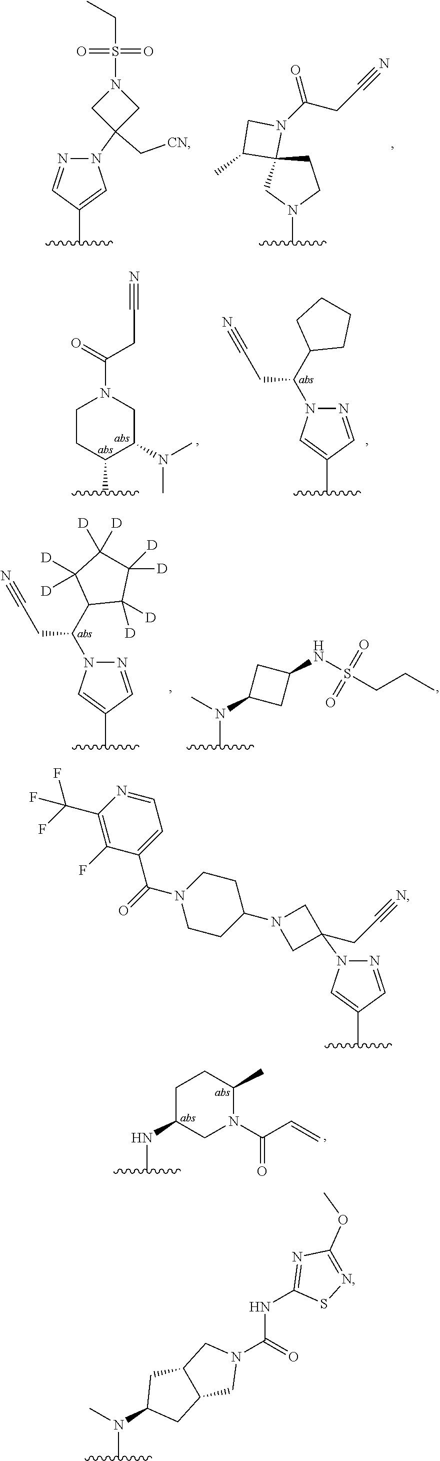

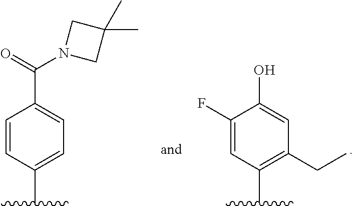 [SMILES: *c1ccc(C(=O)N2CC(C)(C)C2)cc1 ; *c1cc(F)c(O)cc1CC]

In the present application, the R₁ and the R₂ may both be hydrogen atom.

In the present application, the JAK inhibitor may comprise the compound as shown in Formula I, wherein, the R₃ may be selected from amido and 5-10 membered aromatic ring (e.g., 6-membered heteroaromatic ring or 9-membered heteroaromatic ring), the aromatic ring may be a bicyclic ring, and may be substituted by a cyclic group or a chain group.

For example, the R₃ may be amido, the amido may be optionally substituted by cyclobutyl or cyclopropyl. For example, the R₃ may be

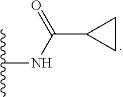 [SMILES: *NC(=O)C1CC1]

For example, the R₃ may be

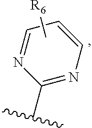

wherein, the R₆ may be

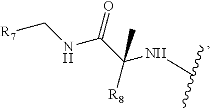

wherein, the R₇ may be selected from —CF₃, —CH₂F, —CH₂F and —CH₃, and R₈ may be selected from C₁-C₃ alkyl. For example, the R₇ may be —CF₃, and the R₈ may be ethyl.

As another example, the R₃ may be

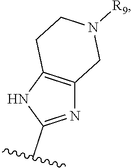

wherein, the R₉ may be 6-membered heterocycle or 6-membered aromatic ring, the ring may be further substituted by C₁-C₃ alkyl. For example, the R₉ may be selected from

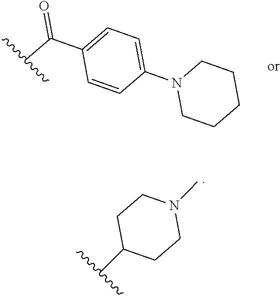

In the present application, the JAK inhibitor may comprise the compound as shown in Formula I, wherein, the compound as shown in Formula I may comprise any one or more of compounds I-1 to 1-15:

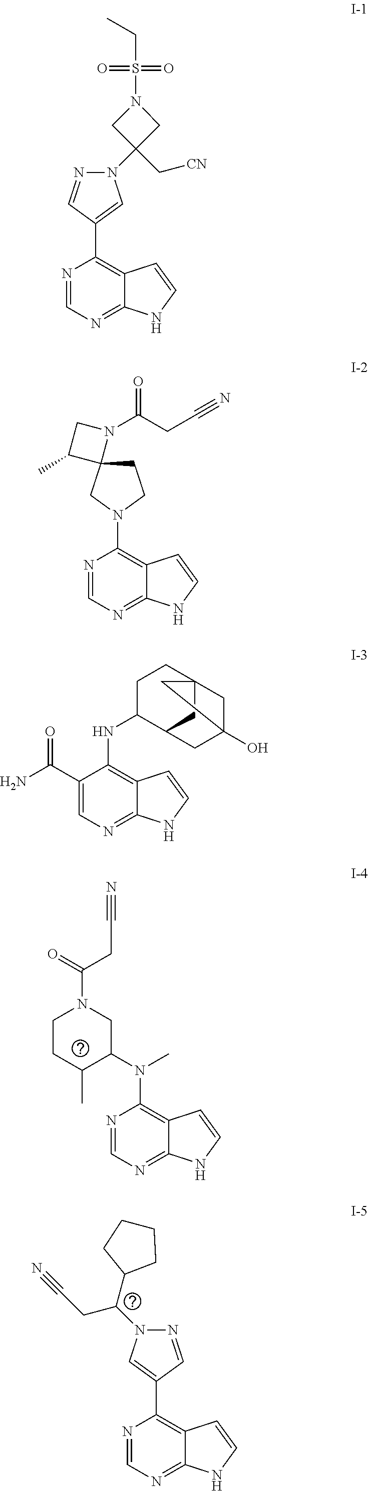 [SMILES: CCS(=O)(=O)N1CC(CC#N)(n2cc(-c3ncnc4[nH]ccc34)cn2)C1 ; C[C@@H]1CN(C(=O)CC#N)[C@@]12CCN(c1ncnc3[nH]ccc13)C2 ; NC(=O)c1cnc2[nH]ccc2c1NC1CCC23C[C@@H]1CC(O)(C2)C3 ; CC1CCN(C(=O)CC#N)CC1N(C)c1ncnc2[nH]ccc12 ; N#CCC(C1CCCC1)n1cc(-c2ncnc3[nH]ccc23)cn1]

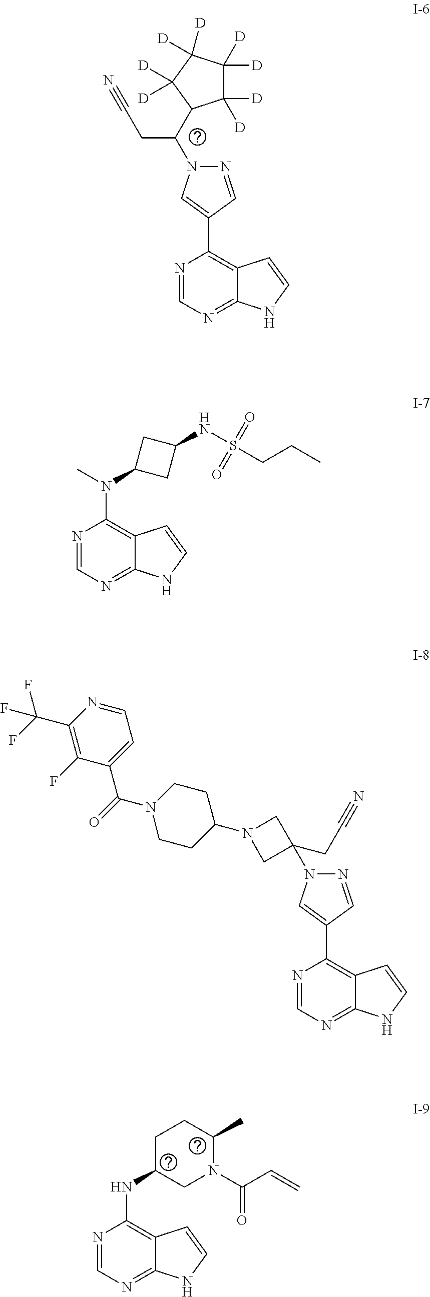 [SMILES: [2H]C1([2H])C(C(CC#N)n2cc(-c3ncnc4[nH]ccc34)cn2)C([2H])([2H])C([2H])([2H])C1([2H])[2H] ; CCCS(=O)(=O)N[C@H]1C[C@@H](N(C)c2ncnc3[nH]ccc23)C1 ; N#CCC1(n2cc(-c3ncnc4[nH]ccc34)cn2)CN(C2CCN(C(=O)c3ccnc(C(F)(F)F)c3F)CC2)C1 ; C=CC(=O)N1C[C@@H](Nc2ncnc3[nH]ccc23)CC[C@H]1C]

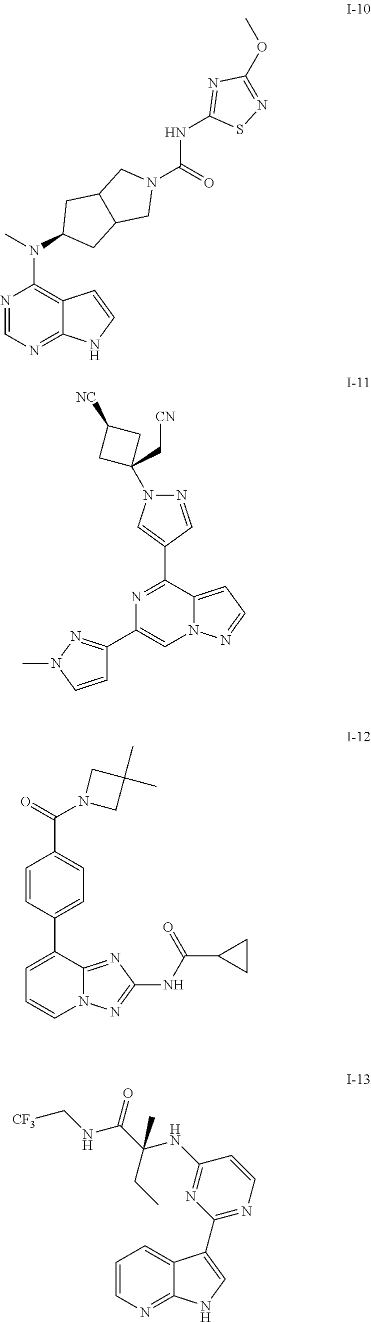

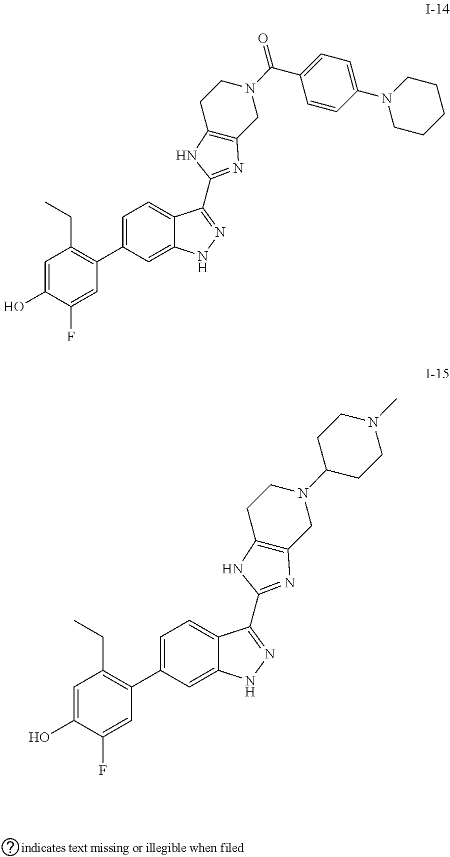 [SMILES: CCc1cc(O)c(F)cc1-c1ccc2c(-c3nc4c([nH]3)CCN(C(=O)c3ccc(N5CCCCC5)cc3)C4)n[nH]c2c1 ; CCc1cc(O)c(F)cc1-c1ccc2c(-c3nc4c([nH]3)CCN(C3CCN(C)CC3)C4)n[nH]c2c1]

In the present application, the JAK inhibitor may comprise a compound as shown in Formula II:

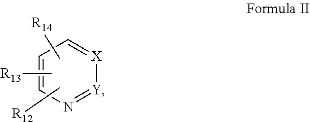

wherein,
the X and Y may be each independently selected from C or N, and the R₁₂, R₁₃, R₁₄ may each independently comprise a group selected from the following group: hydrogen, protium, deuterium, tritium, C₁-C₅ alkyl, halogen, alkoxy, amino, amido, sulfonamido, linear alkyl, cycloalkyl, heterocycloalkyl, alkenyl, alkynyl, aryl and heteroaryl.

For example, in the compound as shown in Formula II, the X may be C, and the Y may be N; or the X may be N, and the Y may be C.

In the present application, the JAK inhibitor may comprise a compound as shown in Formula II-a:

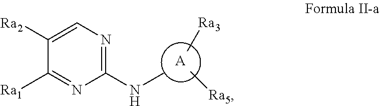

wherein, the Rₐ₁ and Rₐ₂ may comprise any substituents as allowed by valance, ring A may be aromatic ring or heteroaromatic ring optionally substituted by Rₐ₃ and/or Rₐ₅, the Rₐ₃ and Rₐ₅ may be each independently selected from: hydrogen atom, cycloalkyl, heterocycloalkyl, aryl, heteroaryl, alkoxy, or, there is a methyl group between the ring A and —NH—.

For example, the Rₐ₁ may be selected from 4-10-membered aromatic cyclyl, 4-10-membered aromatic heterocyclyl, 4-10-membered cycloalkyl, 4-10-membered heterocycloalkyl, the cyclyl may be further substituted by amido or sulfonyl, the sulfonylamido may be further substituted by cyanogroup, C₁-C₆ alkyl or 5-6-membered heterocyclyl. The ring may be a monocyclic ring or a bicyclic ring.

For example, the Rₐ₁ may be selected from benzene ring, 8-membered heterobicyclic ring and 9-membered heteroaromatic bicyclic ring, wherein the ring may be further substituted, the cyclyl may be further substituted by amido, the amido may be further substituted by C₁-C₃ alkyl, cyclopropyl or 5-membered heterocycle, the C₁-C₃ alkyl, cyclopropyl or 5-membered heterocycle may be further substituted by cyanogroup, fluorine, 6-membered heterocyclyl.

For example, the Rₐ₁ may be amino substituted by 4-10-membered aromatic cyclyl. For example, the Rₐ₁ may be amino substituted by benzene ring or 5-membered heteroaromatic ring, the benzene ring may be further substituted by sulfonyl, the 5-membered heteroaromatic ring may be further substituted by methyl.

For example, in the compound as shown in Formula II-a, the Rₐ₂ may be selected from: hydrogen, C1-C₃ alkyl and halogen.

For example, in the compound as shown in Formula II-a, the Rₐ₂ may be selected from: hydrogen, methyl and chlorine.

In some cases, in the compound as shown in Formula II-a, the ring A may be selected from benzene ring or imidazole ring, the benzene ring or imidazole ring may be optionally substituted by C₁-C₃ alkyl, 5-6-membered heterocycloalkyl, 5-6-membered heteroaryl or halogen, the alkyl or ring may be further substituted by hydroxyl.

In some cases, in the compound as shown in Formula II-a, the Rₐ₁ may be selected from

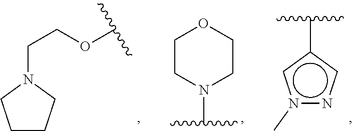

methyl or methoxy, and the Rₐ₅ is hydrogen atom.

In the present application, in the compound as shown in Formula II, the R₁₂, R₁₃, R₁₄ are each independently selected from the following group:

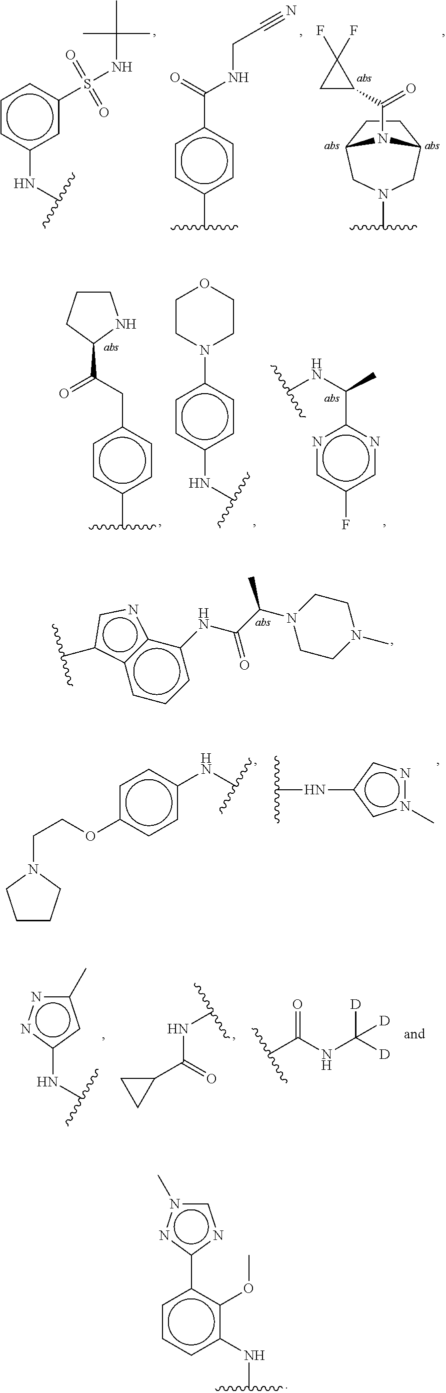

In the present application, the JAK inhibitor may comprise one or more of compounds II-1 to II-7:

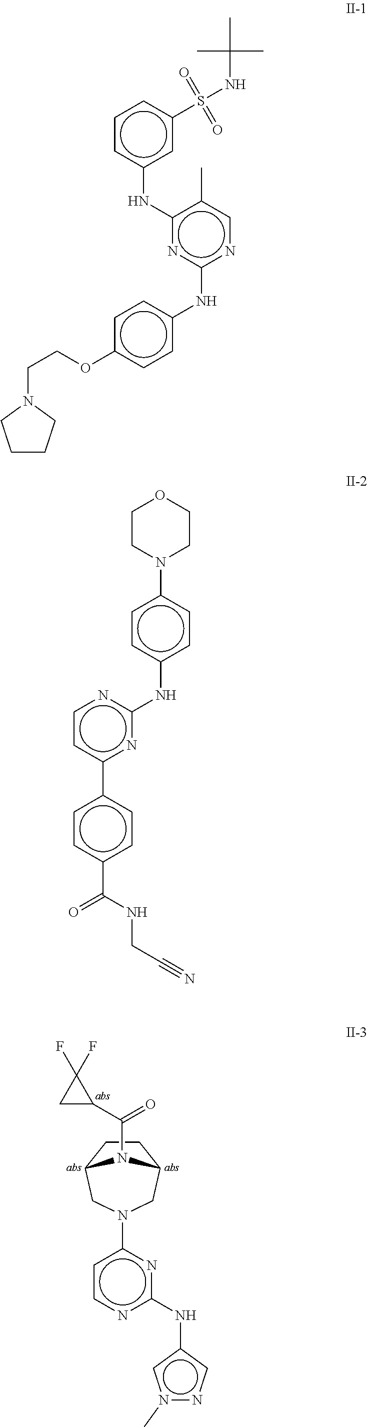

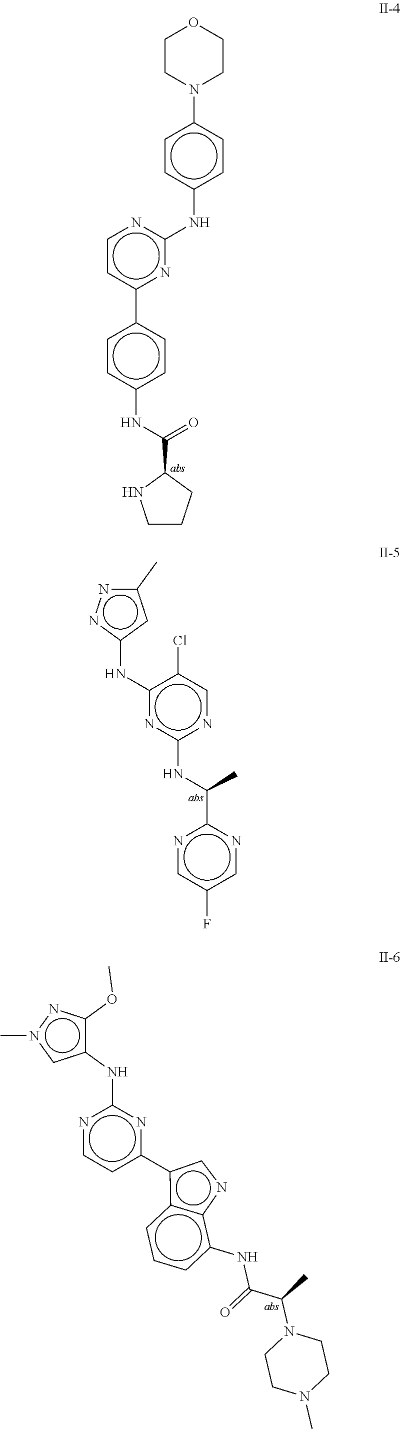

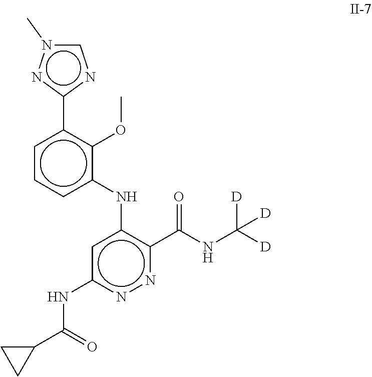

In the present application, the JAK inhibitor may comprise a compound as shown in Formula III:

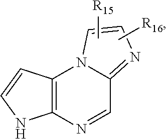

wherein the R₁₅ and R₁₆ are each independently selected from hydrogen atom, cycloalkyl which is optionally substituted by a substituent, heterocycloalkyl which is optionally substituted by a substituent, aryl which is optionally substituted by a substituent and heteroaryl which is optionally substituted by a substituent, the substituent is selected from: amido, alkyl, cycloalkyl, heterocycloalkyl, cyanogroup, amino, hydroxyl and halogen.

For example, the R₁₅ or the R₁₆ may be 4-10-membered heterocycloalkyl, and the heterocycloalkyl is optionally substituted by amido or C₁-C₆ alkyl, the amido may be further substituted by C₁-C₆ alkyl, the alkyl may be further substituted by halogen. For example, the R₁₅ or the R₁₆ may be

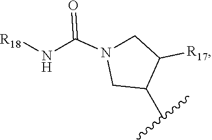

wherein, the R₁₇ and R₁₈ are each independently C₁-C₆ alkyl, and the alkyl may be substituted by halogen. For example, the R₁₇ may be ethyl, and the R₁₈ may be ethyl substituted by fluorine. As another example, the R₁₇ may be ethyl, and the R₁₈ may be —CH₂—CF₃.

For example, one of the R₁₅ or the R₁₆ may be hydrogen atom.

In the present application, the JAK inhibitor may be a compound

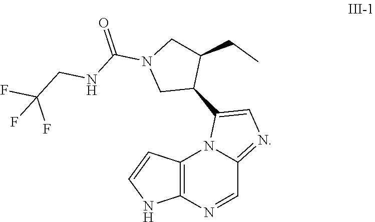 [SMILES: CC[C@@H]1CN(C(=O)NCC(F)(F)F)C[C@@H]1c1cnc2cnc3[nH]ccc3n12]

In the present application, the JAK inhibitor may comprise a compound as shown in Formula IV:

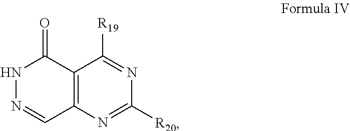

wherein, the R₁₉ and R₂₀ may be each independently selected from hydrogen atom, nitro, 4-10-membered cycloalkyl, 4-10-membered heterocycloalkyl, 4-10-membered aryl and 4-10-membered heteroaryl, wherein the nitro, cycloalkyl, heterocycloalkyl, aryl, heteroaryl may be further substituted by cyanogroup, alkyl, cycloalkyl, heterocycloalkyl or hydroxyl.

For example, the R₁₉ may be nitro, the nitro may be substituted by a substituted benzene ring. As another example, the benzene ring may be substituted by piperidine, the piperidine may be further substituted by hydroxyl. For example, the R₁₉ may be

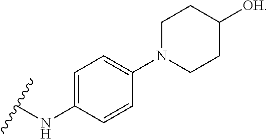 [SMILES: *Nc1ccc(N2CCC(O)CC2)cc1]

For example, the R₂₀ may be piperidinyl, the piperidinyl may be substituted by C₁-C₃ alkyl, the alkyl may be further substituted by cyanogroup or hydroxyl. As another example, the R₂₀ may be piperidinyl, the piperidinyl may be substituted by methyl, the methyl may be further substituted by hydroxyl. As another example, the R₂₀ may be

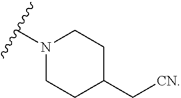 [SMILES: *N1CCC(CC#N)CC1]

In the present application, the JAK inhibitor may be a compound III-1:

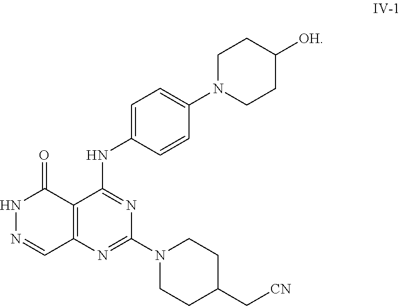 [SMILES: N#CCC1CCN(c2nc(Nc3ccc(N4CCC(O)CC4)cc3)c3c(=O)[nH]ncc3n2)CC1]

Method

The present application provides a method of preventing or treating diseases or disorders associated with antitumor agents, comprising administering to a subject the JAK inhibitor above.

The present application provides a method of preventing or treating EGFR dysfunction-associated rash, comprising administering to a subject the JAK inhibitor above.

The term “prevention” as used herein generally refers to the prevention of occurrence, recurrence, or spread of diseases or one or more symptoms thereof. In the context of the present application, the “prevention” may be interchangeably used with the “preventive treatment”. In some embodiments, the “prevention” generally refers to the treatment of providing a patient suffering from the diseases or disorders as described in the present application with the medicament in accordance with the present application with or without administration of other medicaments as described in the present application prior to the occurrence of any symptoms.

The term “treatment” as used herein generally refers to eliminating or improving one or more symptoms associated with the diseases. In some embodiments, the treatment generally refers to eliminating or ameliorating a disease by administering one or more therapeutic agents to the patients with such disease. In some embodiments, the “treatment” may be administering a medicament in the presence or absence of other therapeutic agent(s) after the occurrence of a particular disease.

The term “subject” as used herein generally refers to a human or non-human animal (including mammals) in need of diagnosing, prognosing, improving, preventing and/or treating diseases, especially those in need of treatment or prevention by using the JAK inhibitors.

In some embodiments, the subject may include a cancer patient.

For example, the cancer patient may have been, is being, and/or will be administered with an antitumor agent. For example, the antitumor agent. may be the antitumor agent. as described herein.

For example, the cancer patient may have been, is being, and/or will be administered with an EGFR inhibitor. For example, the EGFR inhibitor may be the EGFR inhibitor as described herein.

In some embodiments, the subject may be a human or non-human mammal. The non-human mammal may include any mammalian species other than human, e.g., livestocks (e.g., cow, pig, sheep, chick, rabbit or horse), or rodents (e.g., rat and mouse), or primates (e.g., gorilla and monkey), or domestic animals (e.g., dog and cat).

In some embodiments, after administration of the JAK inhibitor of the present application, the severity of the EGFR dysfunction-caused rash of the subject may be relieved. In some embodiments, the relief may be evaluated in accordance with the grading standard of NCI-CTCAE V5.0. For example, the severity grading of the epithelial diseases of the subject was decreased, e.g., from Grade 5 to Grade 1 (e.g., from Grade 5 to Grade 4, from Grade 5 to Grade 3, from Grade 5 to Grade 2, from Grade 4 to Grade 3, from Grade 4 to Grade 2, from Grade 4 to Grade 1, from Grade 3 to Grade 2, from Grade 3 to Grade 1 or from Grade 2 to Grade 1). In some embodiments, the relief generally means that the occurrence or development of the EGFR dysfunction-induced rash in the subject is delayed.

In some embodiments, by administering to a subject in need thereof an effective amount of the JAK inhibitor of the present application, the severity grading of the rash of the subject may be decreased from Grade 5 to Grade 1 (e.g., from Grade 5 to Grade 4, from Grade 5 to Grade 3, from Grade 5 to Grade 2, from Grade 4 to Grade 3, from Grade 4 to Grade 2, from Grade 4 to Grade 1, from Grade 3 to Grade 2, from Grade 3 to Grade 1 or from Grade 2 to Grade 1).

In some embodiments, the JAK inhibitor in the method of the present application may be a compound selected from the following group:

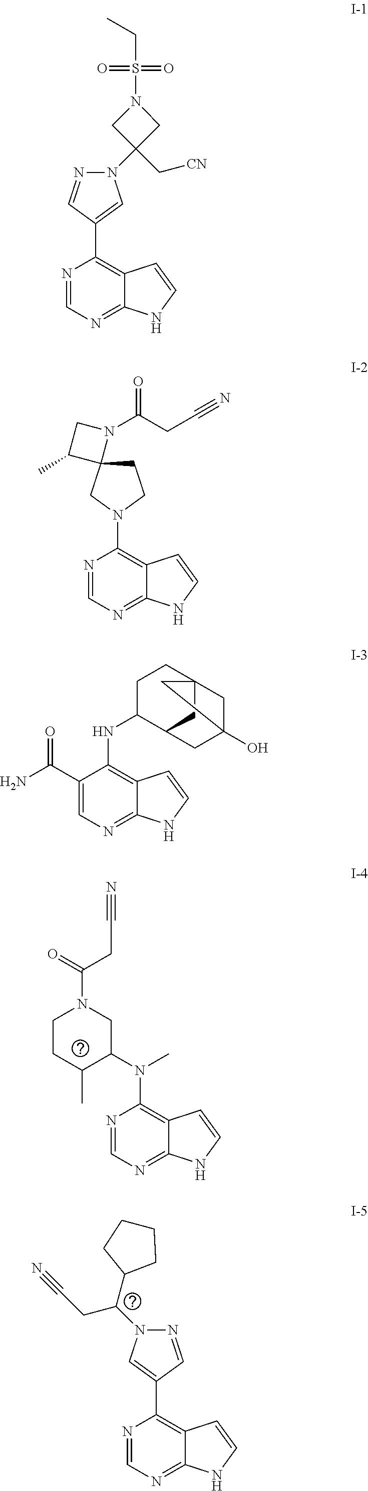 [SMILES: CCS(=O)(=O)N1CC(CC#N)(n2cc(-c3ncnc4[nH]ccc34)cn2)C1 ; C[C@@H]1CN(C(=O)CC#N)[C@@]12CCN(c1ncnc3[nH]ccc13)C2 ; NC(=O)c1cnc2[nH]ccc2c1NC1CCC23C[C@@H]1CC(O)(C2)C3 ; CC1CCN(C(=O)CC#N)CC1N(C)c1ncnc2[nH]ccc12 ; N#CCC(C1CCCC1)n1cc(-c2ncnc3[nH]ccc23)cn1]

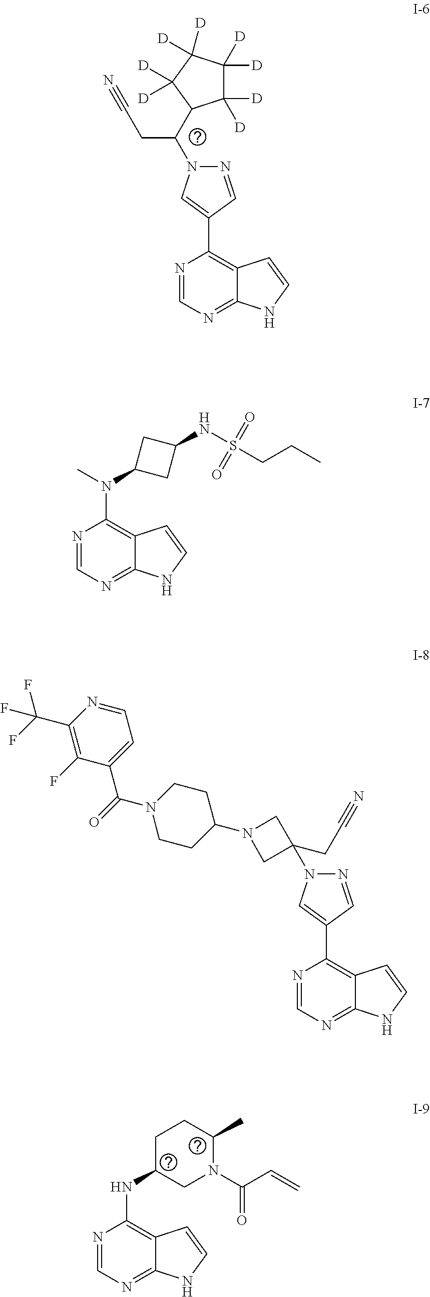 [SMILES: [2H]C1([2H])C(C(CC#N)n2cc(-c3ncnc4[nH]ccc34)cn2)C([2H])([2H])C([2H])([2H])C1([2H])[2H] ; CCCS(=O)(=O)N[C@H]1C[C@@H](N(C)c2ncnc3[nH]ccc23)C1 ; N#CCC1(n2cc(-c3ncnc4[nH]ccc34)cn2)CN(C2CCN(C(=O)c3ccnc(C(F)(F)F)c3F)CC2)C1 ; C=CC(=O)N1C[C@@H](Nc2ncnc3[nH]ccc23)CC[C@H]1C]

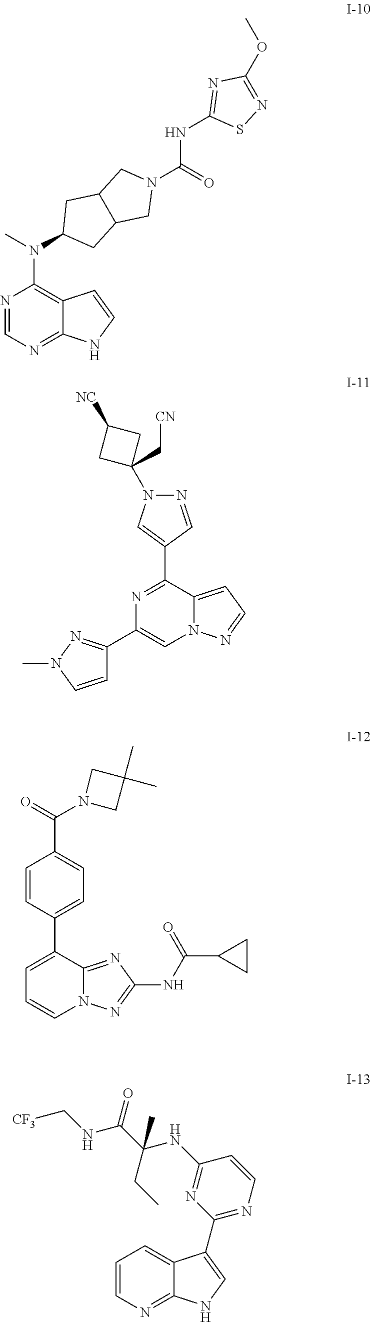

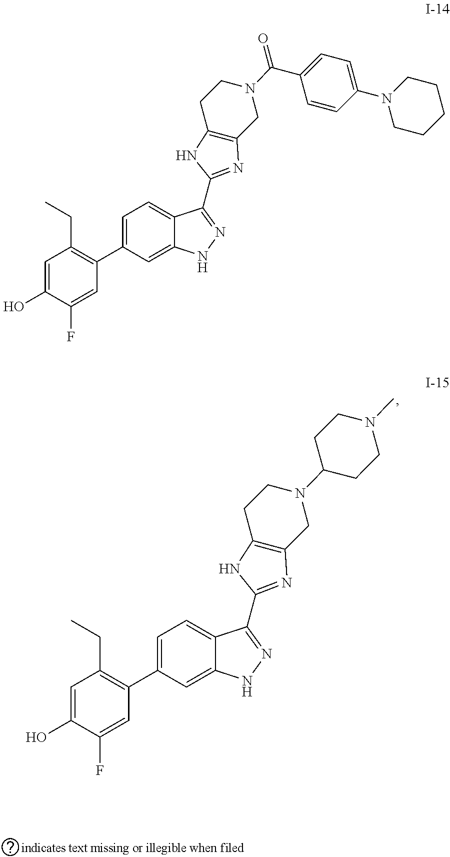 [SMILES: CCc1cc(O)c(F)cc1-c1ccc2c(-c3nc4c([nH]3)CCN(C(=O)c3ccc(N5CCCCC5)cc3)C4)n[nH]c2c1 ; CCc1cc(O)c(F)cc1-c1ccc2c(-c3nc4c([nH]3)CCN(C3CCN(C)CC3)C4)n[nH]c2c1]

which can be used for preventing or treating EGFR dysfunction-associated rash.

In some embodiments, the aforesaid JAK inhibitors can be used to prevent or treat diseases or disorders associated with anti-tumor agents.

In some embodiments, by administering to a subject in need thereof an effective amount of one or more of compound I-1 to compound I-15, the severity grading of the rash of the subject may be decreased from Grade 5 to Grade 1 (e.g., Grade 5 to Grade 4, Grade 5 to Grade 3, Grade 5 to Grade 2, Grade 4 to Grade 3, Grade 4 to Grade 2, Grade 4 to Grade 1, Grade 3 to Grade 2, Grade 3 to Grade 1 or Grade 2 to Grade 1).

In some embodiments, the JAK inhibitor in the method of the present application may be a compound selected from the following group:

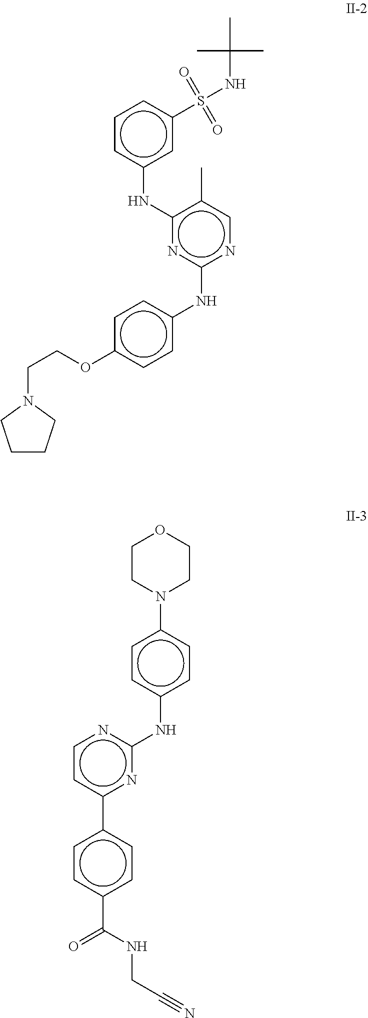

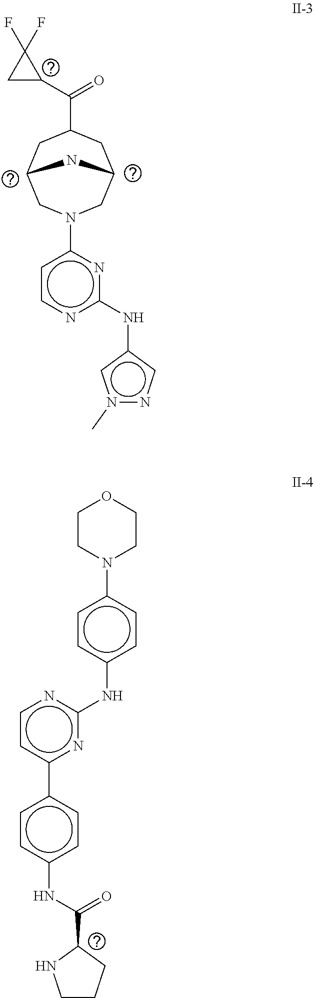

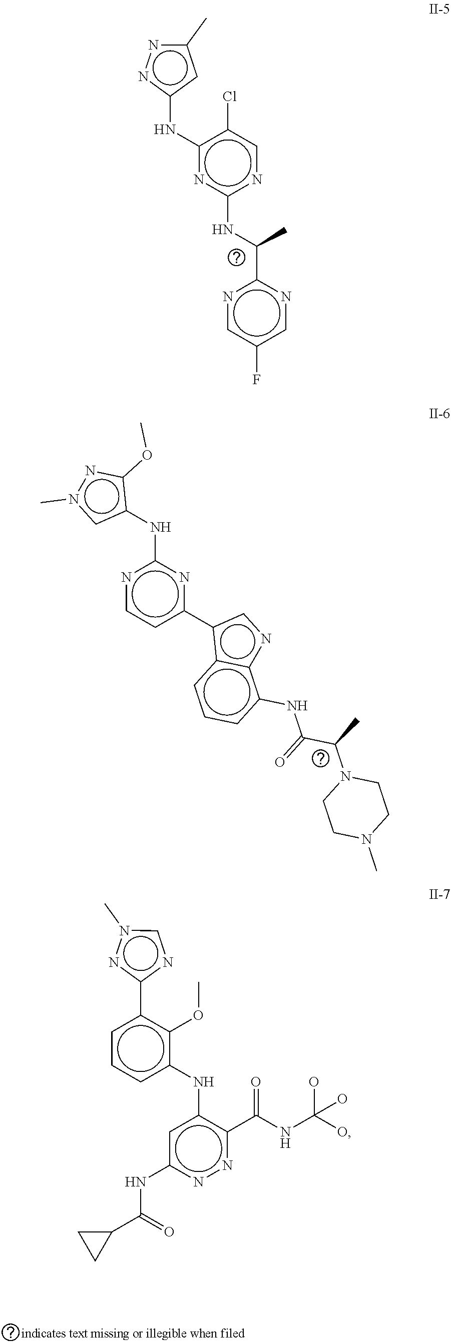

which can be used for preventing or treating EGFR dysfunction-associated rash.

In some embodiments, the aforesaid JAK inhibitors can be used to prevent or treat diseases or disorders associated with anti-tumor agents.

In some embodiments, by administering to a subject in need thereof an effective amount of one or more of compound II-1 to compound II-7, the severity grading of the rash of the subject may be decreased from Grade 5 to Grade 1 (e.g., Grade 5 to Grade 4, Grade 5 to Grade 3, Grade 5 to Grade 2, Grade 4 to Grade 3, Grade 4 to Grade 2, Grade 4 to Grade 1, Grade 3 to Grade 2, Grade 3 to Grade 1 or Grade 2 to Grade 1).

In some embodiments, the JAK inhibitor in the method of the present application may be the compound

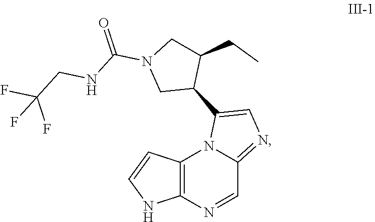 [SMILES: CC[C@@H]1CN(C(=O)NCC(F)(F)F)C[C@@H]1c1cnc2cnc3[nH]ccc3n12]

which can be used for preventing or treating EGFR dysfunction-associated rash.

In some embodiments, the aforesaid JAK inhibitors can be used to prevent or treat diseases or disorders associated with anti-tumor agents.

In some embodiments, by administering to a subject in need thereof an effective amount of the compound III-1, the severity grading of the rash of the subject may be decreased from Grade 5 to Grade 1 (e.g., Grade 5 to Grade 4, Grade 5 to Grade 3, Grade 5 to Grade 2, Grade 4 to Grade 3, Grade 4 to Grade 2, Grade 4 to Grade 1, Grade 3 to Grade 2, Grade 3 to Grade 1 or Grade 2 to Grade 1).

In some embodiments, the JAK inhibitor in the method of the present application may be the compound

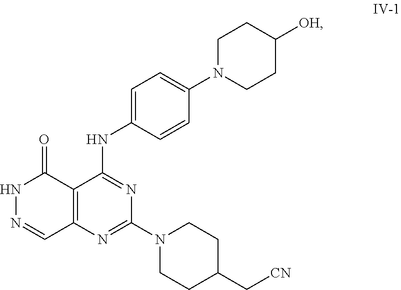 [SMILES: N#CCC1CCN(c2nc(Nc3ccc(N4CCC(O)CC4)cc3)c3c(=O)[nH]ncc3n2)CC1]

which can be used for preventing or treating EGFR dysfunction-associated rash.

In some embodiments, the aforesaid JAK inhibitors can be used to prevent or treat diseases or disorders associated with anti-tumor agents.

In some embodiments, by administering to a subject in need thereof an effective amount of the compound IV-1, the severity grading of the rash of the subject may be decreased from Grade 5 to Grade 1 (e.g., Grade 5 to Grade 4, Grade 5 to Grade 3, Grade 5 to Grade 2, Grade 4 to Grade 3, Grade 4 to Grade 2, Grade 4 to Grade 1, Grade 3 to Grade 2, Grade 3 to Grade 1 or Grade 2 to Grade 1).

The term “effective amount” as used herein generally refers to an amount of medicament capable of ameliorating or eliminating diseases or symptoms of the subject, or preventively inhibiting or prohibiting the occurrence of diseases or symptoms. The effective amount may be an amount of medicament capable of ameliorating one or more diseases or symptoms to some extent in the subject; an amount of medicament capable of partially or completely restoring one or more physiological or biochemical parameters associated with the causes of diseases or symptoms to normal; and/or an amount of medicament capable of reducing the possibility of the occurrence of diseases or symptoms.

The JAK inhibitor as described in the present application may be administered in a manner well known in the art, e.g., by injection (e.g., subcutaneous, intraperitoneal, intraarticular, intraarterial, intrathecal, intrastemal, intrathecal, intralesional, intracranial, intramuscular, intracutaneous and intravenous injection or infusion) or non-injection (e.g., oral, nasal, sublingual, vaginal, rectal, or topical administration). The JAK inhibitor of the present application may be administered in a form of a pharmaceutical combination or a kit.

In the present application, the JAK inhibitor may be prepared for transdermal administration.

In some embodiments, the concentration of the JAK inhibitor as provided in the present application may be about 0.01% (w/w) to about 10% (w/w), e.g., about 0.01% (w/w) to about 9% (w/w), about 0.01% (w/w) to about 8% (w/w), about 0.01% (w/w) to about 7% (w/w), about 0.01% (w/w) to about 6% (w/w), about 0.01% (w/w) to about 5% (w/w), about 0.01% (w/w) to about 4% (w/w), about 0.01% (w/w) to about 3% (w/w), about 0.01% (w/w) to about 2% (w/w), about 0.01% (w/w) to about 1% (w/w), about 0.01% (w/w) to about 0.5% (w/w), about 0.01% (w/w) to about 0.1% (w/w) or about 0.01% (w/w) to about 0.05% (w/w). As another example, the concentration of the JAK inhibitor as provided in the present application may be about 0.02% (w/w) to about 0.05% (w/w), about 0.02% (w/w) to about 1% (w/w), about 0.02% (w/w) to about 2% (w/w), about 0.02% (w/w) to about 5% (w/w), about 0.02% (w/w) to about 0.75% (w/w) or about 0.02% (w/w) to about 1.5% (w/w).

In the present application, the JAK inhibitor may be prepared for local administration.

In some embodiments, the site of the local administration may not be the occurrence site of cancer or potential metastatic site of cancer. For example, the administration site may not be the primary site of cancer. As another example, the administration site may not be a metastatic site of cancer. For example, the metastatic site may comprise sites of cancer metastasis occurrence resulting from lymphatic metastasis, vascular metastasis, and/or implantative metastasis. In some embodiments, the metastatic site may comprise bone, brain, liver, stomach, and/or lung. As another example, the administration site may not be a recurrence site of cancer.

In the present application, the medicament or the JAK inhibitor may be prepared for transdermal administration. In the present application, the medicament or the JAK inhibitor may be prepared for local administration. In some embodiments, the medicament or the JAK inhibitor is prepared for local skin administration. For example, in the present application, the medicament or the JAK inhibitor can be prepared as cream, lotion, gel, ointment, oleamen, spray, liposome preparation, liniment and/or aerosol. For example, in the present application, the transdermal dosage forms prepared from the medicament or the JAK inhibitor may be transdermal preparations in solution (cream, gel, ointment, paste, etc.), and may also be transdermal preparations in suspension (cream, gel, ointment, paste, etc.).

In some embodiments, the JAK inhibitor of the present application may be administered together with an EGFR inhibitor. In some embodiments, the JAK inhibitor may be administered before, simultaneously with, or after the administration of an EGFR inhibitor to the subject. In some embodiments, the JAK inhibitor may be separately administered from the EGFR inhibitor as a part of a multi-dose regimen. In some embodiments, the JAK inhibitor may be simultaneously administered with the EGFR inhibitor. In the embodiments of simultaneous administration, these JAK inhibitors may be a part of a single dosage form, which is mixed with the currently disclosed EGFR inhibitor to form a single composition. In some other embodiments, these JAK inhibitors may be approximately simultaneously administered with the EGFR inhibitor as a separate dose.

In the embodiments in which the JAK inhibitor and the EGFR inhibitor are administered at intervals, the JAK inhibitor may be administered before or after the administration of the EGFR inhibitor at intervals. The time intervals may be 1 min, 2 min, 5 min, 10 min, 20 min, 30 min, 45 min, 1 hr, 2 hrs, 3 hrs, 4 hrs, 5 hrs, 6 hrs, 12 hrs, 18 hrs, 1 day, 2 days, 3 days, 1 week, 2 weeks, 3 weeks, 1 month, 2 months, 3 months or longer.

In some embodiments, the EGFR inhibitor of the present application may be administered in the same administration route as the JAK inhibitor or administered in a different route.

Pharmaceutical Composition and Kit

In some embodiments, the JAK inhibitor or a pharmaceutically acceptable salt thereof may be administered as a part of a medicament or a pharmaceutical combination.

In some embodiments, the medicament may comprise a JAK inhibitor or a pharmaceutically acceptable salt thereof and one or more pharmaceutically acceptable carriers.

In some embodiments, the pharmaceutical combination or kit may comprise 1) an EGFR inhibitor; and 2) a JAK inhibitor or a pharmaceutically acceptable salt thereof. In some embodiments, the EGFR inhibitor and the JAK inhibitor or the pharmaceutically acceptable salt thereof may not be mixed with each other. For example, the EGFR inhibitor and the JAK inhibitor or the pharmaceutically acceptable salt thereof may be each independently present in a separate container. For example, the EGFR inhibitor may be packaged in one reagent bottle, and the JAK inhibitor or the pharmaceutically acceptable salt thereof may be packaged in another reagent bottle.

In the pharmaceutical combination or kit of the present application, the JAK inhibitor in 2) or a pharmaceutically acceptable salt thereof can prevent or treat diseases or disorders caused by the EGFR inhibitor in 1).

In the pharmaceutical combination or kit of the present application, the JAK inhibitor in 2) or a pharmaceutically acceptable salt thereof does not substantially affect the therapeutic effect of the EGFR inhibitor in 1).

In the present application, the term “does not substantially affect” may mean that as compared to the therapeutic effect of using the EGFR inhibitor alone, the use of the JAK inhibitor or a pharmaceutically acceptable salt thereof in 2) of the pharmaceutical combination or kit and the EGFR inhibitor or a pharmaceutically acceptable salt thereof in 1) has a comparable therapeutic effect, or does not produce a significant disadvantage. For example, for any subject, as compared to the therapeutic effect of using the JAK inhibitor or a pharmaceutically acceptable salt thereof alone, the use of the JAK inhibitor in 2) or a pharmaceutically acceptable salt thereof and the EGFR inhibitor in 1) or a pharmaceutically acceptable salt thereof in the pharmaceutical combination or kit contribute to the same degree of tumor size reduction, or the reduction degree is not less than about 5%, not less than about 4%, not less than about 3%, not less than about 2%, not less than about 1%, not less than about 0.5%, not less than about 0.1%, not less than about 0.01%, not less than about 0.001% or less.

In the pharmaceutical combination or kit of the present application, the JAK inhibitor or a pharmaceutically acceptable salt thereof in 2) is administered before, simultaneously with, or after the administration of the EGFR inhibitor in 1).

In another aspect, the present application provides a method, including the following steps:
1) monitoring rashes of a subject who has been administered with an EGFR inhibitor;
2) when the monitoring shows that the subject develops rashes associated with administration of the EGFR inhibitor, administering the JAK inhibitor of the present application or a pharmaceutically acceptable salt thereof to the subject.

Without being limited by any theory, the following examples are only for the purpose of illustrating the fusion protein, preparation method, and use of the present application, and not intended to limit the invention scope of the present application.

### EXAMPLES

### Examples 1: Experiment for Demonstrating the Ability of the JAK Inhibitor in Preventing the Small Molecular EGFR Inhibitor-Induced Rash in Rat Animal Models

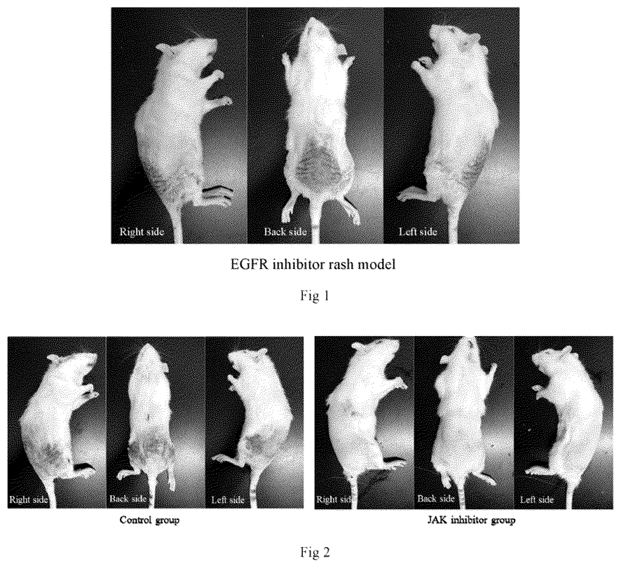

Construction of rat models: small molecular EGFR inhibitors were administered to 6-week female SD rats by daily gavage, and after several days, the rat developed a large area of rash on the back (the photographs are shown in FIG. 1). There was no difference between the left and right sides of the rash area, and the rash degree was similar on both sides. Like in humans, the rats developed rashes on their faces and bodies after oral administration of small molecular EGFR inhibitors. Both have the same cause, and exhibit similar symptoms. Thus, rats are excellent animal models to mimic the rashes induced by the EGFR inhibitors.

After a week of acclimatizing (about 200 g), the SD rats were divided into 10 rats per group. The hair on the back of the rats was gently shaved with an electric shaver on the day before the experiment, and then the intragastric administration was performed. The EGFR inhibitors were dissolved in a sterile aqueous solution and diluted with a PBS buffer solution. The dose was no more than 2 mL per gavage, and the dosages were shown in Table 1. The experiments were divided into a JAK inhibitor group and a control group. After gavage, the back of the rats in the JAK inhibitor group (about 3 cm×3 cm area) was applied with JAK inhibitor ointment (the type and concentration as shown in Table 1); the back of the rats in the control group (about 3 cm×3 cm area) was applied with blank ointment (about 0.5 g). After administration, the rats were fixed by a cylinder for about 4 hrs. Then, the rats were released, wiped with clean water to remove the residual medicament at the application site, and returned to the cage. The gavage frequency of the EGFR inhibitors was shown in Table 1, while the JAK inhibitors and the blank ointment were applied only once a day. The gavage and application tests were repeated every day, until the rats in the control group developed apparent rashes. At this point, the number of rats in the JAK inhibitor group on which the skin kept normal or remarkably milder than the rash in the control group was calculated as the number of rats on which rashes had been effectively inhibited.

Table 1 lists various animal experiment combinations of a small molecular EGFR inhibitors and a JAK inhibitor ointment, and the corresponding experimental results (wherein, the numerical values in the column of control rate=the number of rats in the JAK inhibitor group on which rashes were milder than those of rats in the control group/the total number of rats in the JAK inhibitor group x 100%).

TABLE 1
Experimental conditions and results of Examples 1

| EGFR | Type of the | | | | Concentration | | Control |
|---|---|---|---|---|---|---|---|
| inhibitor | inhibitor | Dosage | Frequency | Administration | wt % | Days | rate |
| Afatinib | Second-generation | 40 mg/kg | Once a day | Tofacitinib | 0.02% | 14 | 20% |
| | EGFR small | | | ointment | | | |
| | molecular | | | | | | |
| | inhibitor | | | | | | |
| Afatinib | Second-generation | 40 mg/kg | Once a day | Tofacitinib | 0.2% | 14 | 40% |
| | EGFR small | | | ointment | | | |
| | molecular | | | | | | |
| | inhibitor | | | | | | |
| Afatinib | Second-generation | 40 mg/kg | Once a day | Tofacitinib | 2% | 14 | 100% |
| | EGFR small | | | ointment | | | |
| | molecular | | | | | | |
| | inhibitor | | | | | | |
| Afatinib | Second-generation | 40 mg/kg | Once a day | Tofacitinib | 5% | 14 | 100% |
| | EGFR small | | | ointment | | | |
| | molecular | | | | | | |
| | inhibitor | | | | | | |
| Afatinib | Second-generation | 40 mg/kg | Once a day | Tofacitinib | 10% | 14 | 100% |
| | EGFR small | | | ointment | | | |
| | molecular | | | | | | |
| | inhibitor | | | | | | |
| Dacomitinib | Second-generation | 10 mg/kg | Once a day | Tofacitinib | 0.02% | 12 | 100% |
| | EGFR small | | | ointment | | | |
| | molecular | | | | | | |
| | inhibitor | | | | | | |
| Dacomitinib | Second-generation | 10 mg/kg | Once a day | Tofacitinib | 0.2% | 12 | 40% |
| | EGFR small | | | ointment | | | |
| | molecular | | | | | | |
| | inhibitor | | | | | | |
| Dacomitinib | Second-generation | 10 mg/kg | Once a day | Tofacitinib | 1% | 12 | 100% |
| | EGFR small | | | ointment | | | |
| | molecular | | | | | | |
| | inhibitor | | | | | | |
| Dacomitinib | Second-generation | 10 mg/kg | Once a day | Tofacitinib | 2% | 12 | 100% |
| | EGFR small | | | ointment | | | |
| | molecular | | | | | | |
| | inhibitor | | | | | | |
| Dacomitinib | Second-generation | 10 mg/kg | Once a day | Tofacitinib | 5% | 12 | 100% |
| | EGFR | | | ointment | | | |
| | small | | | | | | |
| | molecular | | | | | | |
| | inhibitor | | | | | | |
| Dacomitinib | Second-generation | 10 mg/kg | Once a day | Tofacitinib | 10% | 12 | 80% |
| | EGFR small | | | ointment | | | |
| | molecular | | | | | | |
| | inhibitor | | | | | | |
| Afatinib | Second-generation | 40 mg/kg | Once a day | Itacitinib | 0.5% | 14 | 80% |
| | EGFR small | | | ointment | | | |
| | molecular | | | | | | |
| | inhibitor | | | | | | |
| Afatinib | Second-generation | 40 mg/kg | Once a day | Itacitinib | 1.5% | 14 | 100% |
| | EGFR small | | | ointment | | | |
| | molecular | | | | | | |
| | inhibitor | | | | | | |
| Afatinib | Second-generation | 40 mg/kg | Once a day | Itacitinib | 5% | 14 | 80% |
| | EGFR small | | | ointment | | | |
| | molecular | | | | | | |
| | inhibitor | | | | | | |
| Osimertinib | Third-generation | 60 mg/kg | Twice a day | Itacitinib | 0.5% | 10 | 100% |
| | EGFR small | | | ointment | | | |
| | molecular | | | | | | |
| | inhibitor | | | | | | |
| Osimertinib | Third-generation | 60 mg/kg | Twice a day | Itacitinib | 1.5% | 10 | 100% |
| | EGFR small | | | ointment | | | |
| | molecular | | | | | | |
| | inhibitor | | | | | | |
| Osimertinib | Third-generation | 60 mg/kg | Twice a day | Itacitinib | 5% | 10 | 100% |
| | EGFR small | | | ointment | | | |
| | molecular | | | | | | |
| | inhibitor | | | | | | |
| Afatinib | Second-generation | 40 mg/kg | Once a day | Filgotinib | 0.5% | 14 | 100% |
| | EGFR small | | | ointment | | | |
| | molecular | | | | | | |
| | inhibitor | | | | | | |
| Afatinib | Second-generation | 40 mg/kg | Once a day | Filgotinib | 1.5% | 14 | 100% |
| | EGFR small | | | ointment | | | |
| | molecular | | | | | | |
| | inhibitor | | | | | | |
| Afatinib | Second-generation | 40 mg/kg | Once a day | Filgotinib | 5% | 14 | 100% |
| | EGFR | | | ointment | | | |
| | small | | | | | | |
| | molecular | | | | | | |
| | inhibitor | | | | | | |
| EAI045 | Fourth-generation | 80 mg/kg | Once a day | Filgotinib | 0.5% | 11 | 80% |
| | EGFR small | | | ointment | | | |
| | molecular | | | | | | |
| | inhibitor | | | | | | |
| EAI045 | Fourth-generation | 80 mg/kg | Once a day | Filgotinib | 1.5% | 11 | 100% |
| | EGFR small | | | ointment | | | |
| | molecular | | | | | | |
| | inhibitor | | | | | | |
| EAI045 | Fourth-generation | 80 mg/kg | Once a day | Filgotinib | 5% | 11 | 60% |
| | EGFR small | | | ointment | | | |
| | molecular | | | | | | |
| | inhibitor | | | | | | |
| Afatinib | Second-generation | 40 mg/kg | Once a day | Ruxolitinib | 0.05% | 14 | 40% |
| | EGFR small | | | ointment | | | |
| | molecular | | | | | | |
| | inhibitor | | | | | | |
| Afatinib | Second-generation | 40 mg/kg | Once a day | Ruxolitinib | 0.2% | 14 | 40% |
| | EGFR small | | | ointment | | | |
| | molecular | | | | | | |
| | inhibitor | | | | | | |
| Afatinib | Second-generation | 40 mg/kg | Once a day | Ruxolitinib | 0.75% | 14 | 50% |
| | EGFR small | | | ointment | | | |
| | molecular | | | | | | |
| | inhibitor | | | | | | |
| Afatinib | Second-generation | 40 mg/kg | Once a day | Ruxolitinib | 1.5% | 14 | 100% |
| | EGFR small | | | ointment | | | |
| | molecular | | | | | | |
| | inhibitor | | | | | | |
| Afatinib | Second-generation | 40 mg/kg | Once a day | Ruxolitinib | 5% | 14 | 100% |
| | EGFR small | | | ointment | | | |
| | molecular | | | | | | |
| | inhibitor | | | | | | |
| Afatinib | Second-generation | 40 mg/kg | Once a day | Ruxolitinib | 10% | 14 | 80% |
| | EGFR small | | | ointment | | | |
| | molecular | | | | | | |
| | inhibitor | | | | | | |
| Gefitinib | First-generation | 80 mg/kg | Twice a day | Ruxolitinib | 0.05% | 15 | 80% |
| | EGFR small | | | ointment | | | |
| | molecular | | | | | | |
| | inhibitor | | | | | | |
| Gefitinib | First-generation | 80 mg/kg | Twice a day | Ruxolitinib | 0.2% | 15 | 80% |
| | EGFR small | | | ointment | | | |
| | molecular | | | | | | |
| | inhibitor | | | | | | |
| Gefitinib | First-generation | 80 mg/kg | Twice a day | Ruxolitinib | 0.75% | 15 | 100% |
| | EGFR small | | | ointment | | | |
| | molecular | | | | | | |
| | inhibitor | | | | | | |
| Gefitinib | First-generation | 80 mg/kg | Twice a day | Ruxolitinib | 1.5% | 15 | 100% |
| | EGFR small | | | ointment | | | |
| | molecular | | | | | | |
| | inhibitor | | | | | | |
| Gefitinib | First-generation | 80 mg/kg | Twice a day | Ruxolitinib | 5% | 15 | 100% |
| | EGFR small | | | ointment | | | |
| | molecular | | | | | | |
| | inhibitor | | | | | | |
| Gefitinib | First-generation | 80 mg/kg | Twice a day | Ruxolitinib | 10% | 10 | 100% |
| | EGFR small | | | ointment | | | |
| | molecular | | | | | | |
| | inhibitor | | | | | | |
| Erlotinib | First-generation | 70 mg/kg | Once a day | Cerdulatinib | 2% | 15 | 100% |
| | EGFR small | | | ointment | | | |
| | molecular | | | | | | |
| | inhibitor | | | | | | |
| Erlotinib | First-generation | 70 mg/kg | Once a day | Baricitinib | 2% | 15 | 66.7% |
| | EGFR small | | | ointment | | | |
| | molecular | | | | | | |
| | inhibitor | | | | | | |
| Afatinib | Second-generation | 40 mg/kg | Once a day | Fedratinib | 2% | 14 | 75% |
| | EGFR small | | | ointment | | | |
| | molecular | | | | | | |
| | inhibitor | | | | | | |
| Afatinib | Second-generation | 40 mg/kg | Once a day | Upadacitinib | 2% | 14 | 80% |
| | EGFR small | | | ointment | | | |
| | molecular | | | | | | |
| | inhibitor | | | | | | |
| Afatinib | Second-generation | 40 mg/kg | Once a day | Peficitinib | 1% | 14 | 60% |
| | EGFR small | | | ointment | | | |
| | molecular | | | | | | |
| | inhibitor | | | | | | |
| Afatinib | Second-generation | 40 mg/kg | Once a day | TD-1473 | 1% | 14 | 100% |
| | EGFR small | | | ointment | | | |
| | molecular | | | | | | |
| | inhibitor | | | | | | |
| Dacomitinib | Second-generation | 10 mg/kg | Once a day | BMS-986165 | 2% | 12 | 80% |
| | EGFR small | | | ointment | | | |
| | molecular | | | | | | |
| | inhibitor | | | | | | |
| Dacomitinib | Second-generation | 10 mg/kg | Once a day | TYK2 | 2% | 12 | 100% |
| | EGFR small | | | inhibitor | | | |
| | molecular | | | ointment | | | |
| | inhibitor | | | | | | |
| Dacomitinib | Second-generation | 10 mg/kg | Once a day | Ritlecitinib | 1% | 12 | 75% |
| | EGFR small | | | ointment | | | |
| | molecular | | | | | | |
| | inhibitor | | | | | | |
| Osimertinib | Third-generation | 60 mg/kg | Twice a day | Abrocitinib | 2% | 10 | 100% |
| | EGFR small | | | ointment | | | |
| | molecular | | | | | | |
| | inhibitor | | | | | | |
| Osimertinib | Third-generation | 60 mg/kg | Twice a day | Momelotinib | 2% | 10 | 100% |
| | EGFR small | | | ointment | | | |
| | molecular | | | | | | |
| | inhibitor | | | | | | |
| Osimertinib | Third-generation | 60 mg/kg | Twice a day | Brepocitinib | 2% | 10 | 80% |
| | EGFR small | | | ointment | | | |
| | molecular | | | | | | |
| | inhibitor | | | | | | |

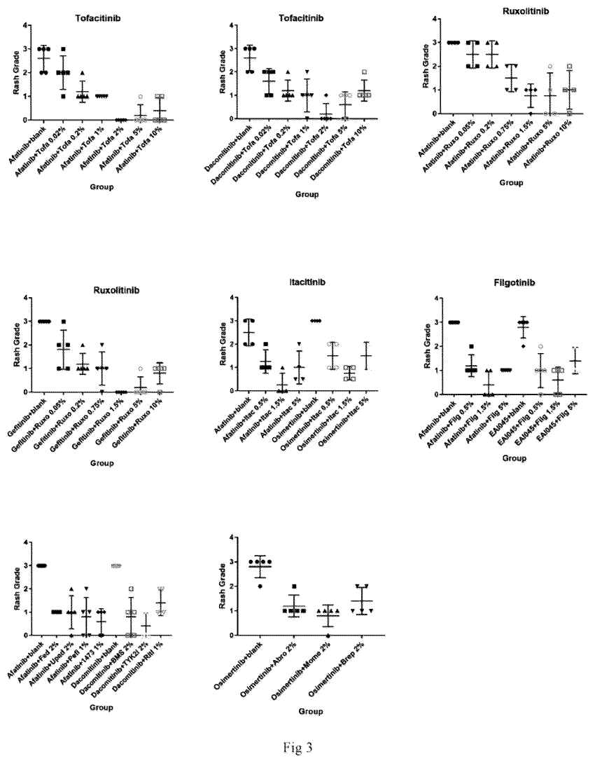

FIG. 2 shows the photographs of left side, back side, and right side of typical rats in the control group and the JAK inhibitor group in Table 1. FIG. 3 shows the rash grading of the JAK inhibitor group and the control group at the end of the experiment.

It can be seen from the results in Table 1 and FIGS. 2-3 that: the JAK inhibitor ointment can effectively prevent rashes caused by a small molecular EGFR inhibitor.

### Examples 2: Experiment for Demonstrating the Ability of the JAK Inhibitor in Preventing the Monoclonal Antibody EGFR Inhibitors-Induced Rashes in Rat Models

After a week of acclimatizing (about 200 g), the SD rats were divided into 10 rats per group. The hair on the back of the rats was gently shaved with an electric shaver on the day before the experiment, and then the administration was performed. The experiments were divided into a JAK inhibitor group and a control group. An EGFR monoclonal antibody solution diluted with normal saline was injected twice a week into the tail vein of rats, for which the injection rate and time were shown in Table 2. After injection, the back of the rats in the JAK inhibitor group (about 3 cm×3 cm area) was applied with JAK inhibitor ointment every day; the back of the rats in the control group (about 3 cm×3 cm area) was applied with blank ointment (about 0.5 g). After applying the medicament, the rats were fixed by a cylinder for about 4 hrs. Then, the rats were released, wiped with clean water to remove the residual medicament at the application site, and returned to the cage. The rats were subjected to tail vein injection twice a week, and were applied with the ointment once a day, until the rats in the control group developed apparent rashes. The number of rats in the JAK inhibitor group on which the skin kept normal or remarkably milder than the rashes of the rats in the control group 10-14 days after the application was recorded and calculated as the number of rats on which rashes had been effectively inhibited.

Table 2 lists various animal experiment combinations of a monoclonal antibody EGFR inhibitor and a JAK inhibitor ointment, and the corresponding experimental results (wherein, the numerical values in the column of control rate=the number of rats in the JAK inhibitor group on which rashes were milder than those of rats in the control group/the total number of rats in the JAK inhibitor group x 100%).

TABLE 2
Experimental conditions and results of Examples 2

| | Type | | | | | | |
|---|---|---|---|---|---|---|---|
| | of the | | | | | | Control |
| EGFR inhibitor | inhibitor | Dosage | Frequency | Administration | Concentration | Days | rate |
| Cetuximab | Monoclonal | 100 mg/kg, 15 min | Tail vein | Ruxolitinib | 5% | 10 | 66.7% |
| | antibody | with an injection rate | injection | ointment | | | |
| | | of 1.3 ml/kg/min for | twice a | | | | |
| | | each time | week | | | | |
| Panitumumab | Monoclonal | 10 mg/kg | Tail vein | Tofacitinib | 2% | 14 | 80% |
| | antibody | | injection | ointment | | | |
| | | | twice a | | | | |
| | | | week | | | | |

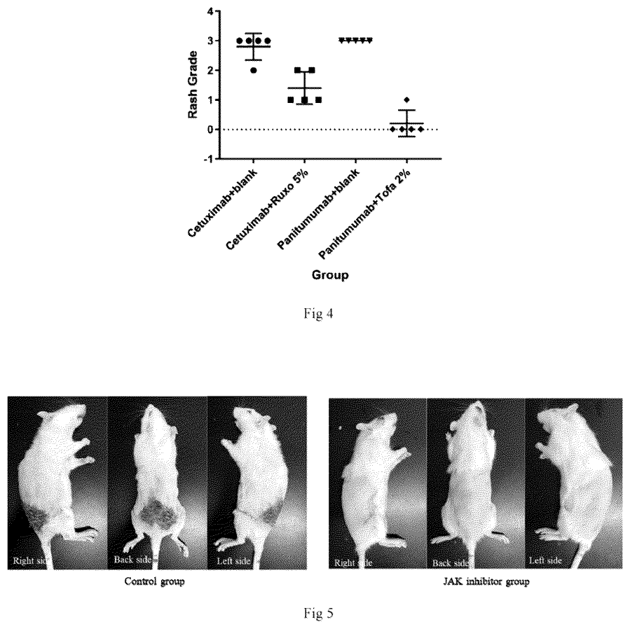

FIG. 4 shows the rash grading of the JAK inhibitor group and the control group (a monoclonal antibody EGFR inhibitor) at the end of the experiment.

It can be seen from the results in Table 2 and FIG. 4 that: the JAK inhibitor ointment can effectively prevent rashes caused by monoclonal antibody EGFR inhibitors.

### Examples 3: Experiment for Demonstrating the Ability of the JAK Inhibitor in Treating the Small Molecular EGFR Inhibitor-Induced Rash in Rat Models

After a week of acclimatizing (about 200 g), the SD rats were divided into 10 rats per group. The hair on the back of the rats was gently shaved with an electric shaver on the day before the experiment, and then the intragastric administration was initiated. The EGFR inhibitors were dissolved in a sterile aqueous solution and diluted with a PBS buffer solution. The dose was no more than 2 mL per gavage, and the dosages were shown in Table 3. The gavage was performed every day, until the rats developed the symptom of rashes, and at this time the therapeutic experiments were performed. The experiments were divided into a JAK inhibitor group and a control group. During the therapeutic experiment, the rats were continuously subjected to gavage with the EGFR inhibitor every day. After gavage, the back of the rats in the JAK inhibitor group (about 3 cm×3 cm area) was applied with JAK inhibitor ointment; the back of the rats in the control group (about 3 cm×3 cm area) was applied with blank ointment. After administration, the rats were fixed by a cylinder for about 4 hrs. Then, the rats were released, wiped with clean water to remove the residual medicament at the application site, and returned to the cage. The gavage frequency of the EGFR inhibitors was shown in Table 3, while the JAK inhibitors and the blank ointment were administered only once a day. The oral gavage of EGFR inhibitors was repeated every day. The number of rats in the JAK inhibitor group on which the skin returned to normal or remarkably milder than the rashes of the rats in the control group was calculated as the number of rats on which rashes had been effectively treated.

Table 3 lists various animal experiment combinations of a small molecular EGFR inhibitor and a JAK inhibitor ointment, and the corresponding experimental results (wherein, the numerical values in the column of control rate=the number of rats in the JAK inhibitor group on which rashes were milder than those of rats in the control group/the total number of rats in the JAK inhibitor group x 100%).

TABLE 3
Experimental conditions and results of Examples 3

| | | | | Modeling | | | |
|---|---|---|---|---|---|---|---|
| EGFR | Type of the | | | days for | | Concentration | Control |
| inhibitor | inhibitor | Dosage | Frequency | Administration | Administration | wt % | rate |
| Afatinib | Second-generation | 40 mg/kg | Once a day | 6 | Tofacitinib | 0.02% | 40% |
| | EGFR small | | | | ointment | | |
| | molecular | | | | | | |
| | inhibitor | | | | | | |
| Afatinib | Second-generation | 40 mg/kg | Once a day | 6 | Tofacitinib | 0.2% | 60% |
| | EGFR small | | | | ointment | | |
| | molecular | | | | | | |
| | inhibitor | | | | | | |
| Afatinib | Second-generation | 40 mg/kg | Once a day | 6 | Tofacitinib | 1% | 80% |
| | EGFR small | | | | ointment | | |
| | molecular | | | | | | |
| | inhibitor | | | | | | |
| Afatinib | Second-generation | 40 mg/kg | Once a day | 6 | Tofacitinib | 2% | 80% |
| | EGFR small | | | | ointment | | |
| | molecular | | | | | | |
| | inhibitor | | | | | | |
| Afatinib | Second-generation | 40 mg/kg | Once a day | 6 | Tofacitinib | 5% | 80% |
| | EGFR small | | | | ointment | | |
| | molecular | | | | | | |
| | inhibitor | | | | | | |
| Afatinib | Second-generation | 40 mg/kg | Once a day | 6 | Tofacitinib | 10% | 75% |
| | EGFR small | | | | ointment | | |
| | molecular | | | | | | |
| | inhibitor | | | | | | |
| Dacomitinib | Second-generation | 10 mg/kg | Once a day | 5 | Tofacitinib | 0.02% | 80% |
| | EGFR small | | | | ointment | | |
| | molecular | | | | | | |
| | inhibitor | | | | | | |
| Dacomitinib | Second-generation | 10 mg/kg | Once a day | 5 | Tofacitinib | 0.2% | 80% |
| | EGFR small | | | | ointment | | |
| | molecular | | | | | | |
| | inhibitor | | | | | | |
| Dacomitinib | Second-generation | 10 mg/kg | Once a day | 5 | Tofacitinib | 1% | 80% |
| | EGFR small | | | | ointment | | |
| | molecular | | | | | | |
| | inhibitor | | | | | | |
| Dacomitinib | Second-generation | 10 mg/kg | Once a day | 5 | Tofacitinib | 2% | 90% |
| | EGFR | | | | ointment | | |
| | small | | | | | | |
| | molecular | | | | | | |
| | inhibitor | | | | | | |
| Dacomitinib | Second-generation | 10 mg/kg | Once a day | 5 | Tofacitinib | 5% | 90% |
| | EGFR small | | | | ointment | | |
| | molecular | | | | | | |
| | inhibitor | | | | | | |
| Dacomitinib | Second-generation | 10 mg/kg | Once a day | 5 | Tofacitinib | 10% | 75% |
| | EGFR small | | | | ointment | | |
| | molecular | | | | | | |
| | inhibitor | | | | | | |
| Afatinib | Second-generation | 40 mg/kg | Once a day | 6 | Ruxolitinib | 0.05% | 20% |
| | EGFR small | | | | ointment | | |
| | molecular | | | | | | |
| | inhibitor | | | | | | |
| Afatinib | Second-generation | 40 mg/kg | Once a day | 6 | Ruxolitinib | 0.2% | 40% |
| | EGFR small | | | | ointment | | |
| | molecular | | | | | | |
| | inhibitor | | | | | | |
| Afatinib | Second-generation | 40 mg/kg | Once a day | 6 | Ruxolitinib | 0.75% | 60% |
| | EGFR small | | | | ointment | | |
| | molecular | | | | | | |
| | inhibitor | | | | | | |
| Afatinib | Second-generation | 40 mg/kg | Once a day | 6 | Ruxolitinib | 1.5% | 100% |
| | EGFR small | | | | ointment | | |
| | molecular | | | | | | |
| | inhibitor | | | | | | |
| Afatinib | Second-generation | 40 mg/kg | Once a day | 6 | Ruxolitinib | 5% | 100% |
| | EGFR small | | | | ointment | | |
| | molecular | | | | | | |
| | inhibitor | | | | | | |
| Afatinib | Second-generation | 40 mg/kg | Once a day | 6 | Ruxolitinib | 10% | 100% |
| | EGFR small | | | | ointment | | |
| | molecular | | | | | | |
| | inhibitor | | | | | | |
| Gefitinib | First-generation | 80 mg/kg | Twice a day | 10 | Ruxolitinib | 0.05% | 33.3% |
| | EGFR small | | | | ointment | | |
| | molecular | | | | | | |
| | inhibitor | | | | | | |
| Gefitinib | First-generation | 80 mg/kg | Twice a day | 10 | Ruxolitinib | 0.2% | 50% |
| | EGFR small | | | | ointment | | |
| | molecular | | | | | | |
| | inhibitor | | | | | | |
| Gefitinib | First-generation | 80 mg/kg | Twice a day | 10 | Ruxolitinib | 0.75% | 80% |
| | EGFR small | | | | ointment | | |
| | molecular | | | | | | |
| | inhibitor | | | | | | |
| Gefitinib | First-generation | 80 mg/kg | Twice a day | 10 | Ruxolitinib | 1.5% | 100% |
| | EGFR small | | | | ointment | | |
| | molecular | | | | | | |
| | inhibitor | | | | | | |
| Gefitinib | First-generation | 80 mg/kg | Twice a day | 10 | Ruxolitinib | 5% | 100% |
| | EGFR small | | | | ointment | | |
| | molecular | | | | | | |
| | inhibitor | | | | | | |
| Gefitinib | First-generation | 80 mg/kg | Twice a day | 10 | Ruxolitinib | 10% | 80% |
| | EGFR small | | | | ointment | | |
| | molecular | | | | | | |
| | inhibitor | | | | | | |
| Afatinib | Second-generation | 40 mg/kg | Once a day | 6 | Itacitinib | 0.5% | 66.7% |
| | EGFR small | | | | ointment | | |
| | molecular | | | | | | |
| | inhibitor | | | | | | |
| Afatinib | Second-generation | 40 mg/kg | Once a day | 6 | Itacitinib | 1.5% | 66.7% |
| | EGFR small | | | | ointment | | |
| | molecular | | | | | | |
| | inhibitor | | | | | | |
| Afatinib | Second-generation | 40 mg/kg | Once a day | 6 | Itacitinib | 5% | 80% |
| | EGFR small | | | | ointment | | |
| | molecular | | | | | | |
| | inhibitor | | | | | | |
| Osimertinib | Third-generation | 60 mg/kg | Twice a day | 7 | Itacitinib | 0.5% | 33.3% |
| | EGFR small | | | | ointment | | |
| | molecular | | | | | | |
| | inhibitor | | | | | | |
| Osimertinib | Third-generation | 60 mg/kg | Twice a day | 7 | Itacitinib | 1.5% | 50% |
| | EGFR small | | | | ointment | | |
| | molecular | | | | | | |
| | inhibitor | | | | | | |
| Osimertinib | Third-generation | 60 mg/kg | Twice a day | 7 | Itacitinib | 5% | 66.7% |
| | EGFR small | | | | ointment | | |
| | molecular | | | | | | |
| | inhibitor | | | | | | |
| Afatinib | Second-generation | 40 mg/kg | Once a day | 6 | Filgotinib | 0.5% | 66.7% |
| | EGFR small | | | | ointment | | |
| | molecular | | | | | | |
| | inhibitor | | | | | | |
| Afatinib | Second-generation | 40 mg/kg | Once a day | 6 | Filgotinib | 1.5% | 66.7% |
| | EGFR small | | | | ointment | | |
| | molecular | | | | | | |
| | inhibitor | | | | | | |
| Afatinib | Second-generation | 40 mg/kg | Once a day | 6 | Filgotinib | 5% | 100% |
| | EGFR small | | | | ointment | | |
| | molecular | | | | | | |
| | inhibitor | | | | | | |
| EAI045 | Fourth-generation | 80 mg/kg | Once a day | 8 | Filgotinib | 0.5% | 75% |
| | EGFR small | | | | ointment | | |
| | molecular | | | | | | |
| | inhibitor | | | | | | |
| EAI045 | Fourth-generation | 80 mg/kg | Once a day | 8 | Filgotinib | 1.5% | 75% |
| | EGFR small | | | | ointment | | |
| | molecular | | | | | | |
| | inhibitor | | | | | | |
| EAI045 | Fourth-generation | 80 mg/kg | Once a day | 8 | Filgotinib | 5% | 90% |
| | EGFR small | | | | ointment | | |
| | molecular | | | | | | |
| | inhibitor | | | | | | |
| Erlotinib | First-generation | 70 mg/kg | Once a day | 10 | Cerdulatinib | 2% | 60% |
| | EGFR small | | | | ointment | | |
| | molecular | | | | | | |
| | inhibitor | | | | | | |
| Erlotinib | First-generation | 70 mg/kg | Once a day | 10 | Baricitinib | 2% | 75% |
| | EGFR small | | | | ointment | | |
| | molecular | | | | | | |
| | inhibitor | | | | | | |
| Afatinib | Second-generation | 40 mg/kg | Once a day | 6 | Fedratinib | 2% | 66.7% |
| | EGFR small | | | | ointment | | |
| | molecular | | | | | | |
| | inhibitor | | | | | | |
| Afatinib | Second-generation | 40 mg/kg | Once a day | 6 | Upadacitinib | 2% | 80% |
| | EGFR small | | | | ointment | | |
| | molecular | | | | | | |
| | inhibitor | | | | | | |
| Afatinib | Second-generation | 40 mg/kg | Once a day | 6 | Peficitinib | 1% | 75% |
| | EGFR small | | | | ointment | | |
| | molecular | | | | | | |
| | inhibitor | | | | | | |
| Afatinib | Second-generation | 40 mg/kg | Once a day | 6 | TD-1473 | 1% | 66.7% |
| | EGFR small | | | | ointment | | |
| | molecular | | | | | | |
| | inhibitor | | | | | | |
| Dacomitinib | Second-generation | 10 mg/kg | Once a day | 5 | BMS-986165 | 2% | 80% |
| | EGFR small | | | | ointment | | |
| | molecular | | | | | | |
| | inhibitor | | | | | | |
| Dacomitinib | Second-generation | 10 mg/kg | Once a day | 5 | TYK2 | 2% | 66.7% |
| | EGFR small | | | | inhibitor | | |
| | molecular | | | | ointment | | |
| | inhibitor | | | | | | |
| Dacomitinib | Second-generation | 10 mg/kg | Once a day | 5 | Ritlecitinib | 1% | 66.7% |
| | EGFR small | | | | ointment | | |
| | molecular | | | | | | |
| | inhibitor | | | | | | |
| Osimertinib | Third-generation | 60 mg/kg | Twice a day | 5 | Abrocitinib | 2% | 80% |
| | EGFR small | | | | ointment | | |
| | molecular | | | | | | |
| | inhibitor | | | | | | |
| Osimertinib | Third-generation | 60 mg/kg | Twice a day | 7 | Momelotinib | 2% | 66.7% |
| | EGFR small | | | | ointment | | |
| | molecular | | | | | | |
| | inhibitor | | | | | | |
| Osimertinib | Third-generation | 60 mg/kg | Twice a day | 7 | Brepocitinib | 2% | 60% |
| | EGFR small | | | | ointment | | |
| | molecular | | | | | | |
| | inhibitor | | | | | | |

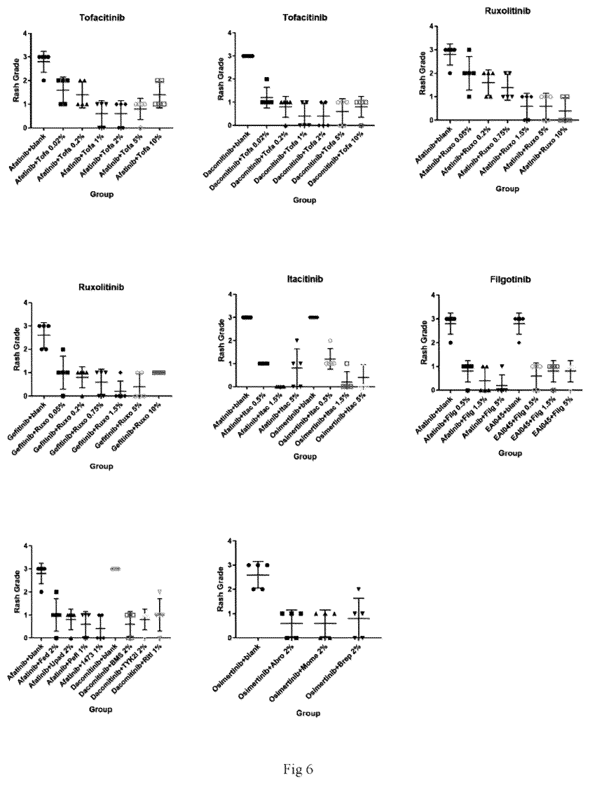

FIG. 5 shows the photographs of left side, back side, and right side of typical rats in the control group and the JAK inhibitor group in Table 3. FIG. 6 shows the rash grading of the JAK inhibitor group and the control group at the end of the experiment.

It can be seen from the results in Table 3 and FIGS. 5-6 that: the JAK inhibitor ointment can effectively treat rashes caused by a small molecular EGFR inhibitor.

### Examples 4: Experiment for Demonstrating the Ability of the JAK Inhibitor in Treating the Monoclonal Antibody EGFR Inhibitors-Induced Rashes in Rat Models

After a week of acclimatizing (about 200 g), the SD rats were divided into 10 rats per group. The hair on the back of the rats was gently shaved with an electric shaver on the day before the experiment, and then the administration was performed. An EGFR monoclonal antibody solution diluted with normal saline was injected twice a week into the tail vein of rats, for which the injection rate and time were shown in Table 4. The rats were continuously administered for 1 to 2 weeks, until the rats developed rashes, and at this time the therapeutic experiments were initiated. The experiments were divided into a JAK inhibitor group and a control group. During the therapeutic experiment, the rats were continuously subjected to injection with the monoclonal antibody EGFR inhibitor twice a week. The back of the rats in the JAK inhibitor group (about 3 cm×3 cm area) was applied with JAK inhibitor ointment every day; and the back of the rats in the control group (about 3 cm×3 cm area) was applied with blank ointment every day. After administration, the rats were fixed by a cylinder for about 4 hrs. Then, the rats were released, wiped with clean water to remove the residual medicament at the application site, and returned to the cage. After administration for 8-10 days, the number of rats in the JAK inhibitor group on which the skin kept normal or remarkably milder than the rashes of the rats in the control group was calculated as the number of rats on which rashes had been effectively inhibited.

Table 4 lists various animal experiment combinations of a monoclonal antibody EGFR inhibitor and a JAK inhibitor ointment, and the corresponding experimental results (wherein, the numerical values in the column of control rate=the number of rats in the JAK inhibitor group on which rashes were milder than those of rats in the control group/the total number of rats in the JAK inhibitor group x 100%).

TABLE 4
Experimental conditions and results of Examples 4

| | | | | Adminis- | | | Days for | |
|---|---|---|---|---|---|---|---|---|
| EGFR | Type of the | | | tration | Adminis- | Concen- | Adminis- | Ameliorating |
| inhibitor | inhibitor | Dosage | Frequency | period | tration | tration | tration | Rate |
| Cetuximab | Monoclonal | 100 mg/kg | Tail | 3-4 | Ruxolitinib | 0.75% | 10 | 100% |
| | antibody | with an injection rate | vein | weeks | ointment | | | |
| | | of 1.3 ml/kg/min for | injection | (6-8 | | | | |
| | | each time | twice | times) | | | | |
| | | | a week | | | | | |
| Cetuximab | Monoclonal | 100 mg/kg | Tail | 3-4 | | | | |
| | antibody | with an injection rate | vein | weeks | Baricitinib | 2% | 10 | 80% |
| | | of 1.3 ml/kg/min for | injection | (6-8 | ointment | | | |
| | | each time | twice | times) | | | | |
| | | | a week | | | | | |
| Panitumumab | Monoclonal | 6 mg/kg, 90 min | Tail | 3-4 | Filgotinib | 5% | 8 | 80% |
| | antibody | | vein | weeks | ointment | | | |
| | | | injection | (6-8 | | | | |
| | | | twice | times) | | | | |
| | | | a week | | | | | |

FIG. 7 shows the rash grading of the JAK inhibitor group and the control group (a monoclonal antibody EGFR inhibitor) at the end of the experiment.

It can be seen from the results in Table 4 and FIG. 7 that: the JAK inhibitor ointment can effectively treat rashes caused by monoclonal antibody EGFR inhibitors.

### Examples 5: Comparison of the JAK Inhibitor Ointment with Other Currently Clinically Available Dermatological Medicaments in the Experiment of Preventing Small Molecular EGFR Inhibitors-Induced Rashes

After a week of acclimatizing (about 200 g), the rats were divided into 10 rats per group. The hair on the back of the rats was gently shaved with an electric shaver on the day before the experiment, and then the intragastric administration was performed. The EGFR inhibitors were dissolved in a sterile aqueous solution and diluted with a PBS buffer solution. The dose was no more than 2 mL per gavage, and the dosages were shown in Table 5. The experiments were divided into a JAK inhibitor group and other dermatologic medicament group. After gavage, the back of the rats in the JAK inhibitor group (about 3 cm×3 cm area) was applied with JAK inhibitor ointment; the back of the rats in the other dermatologic medicament group (about 3 cm×3 cm area) was applied with currently clinically available dermatological medicaments respectively (examples 5). After administration, the rats were fixed by a cylinder for about 4 hrs. Then, the rats were released, wiped with clean water to remove the residual medicament at the application site, and returned to the cage. The gavage frequency of the EGFR inhibitors was shown in Table 5, while the currently clinically available dermatological medicaments and the JAK inhibitors were administered only once. The oral gavage of EGFR inhibitors and topical administration of medicaments on the back were repeated every day, until the rats in the other dermatologic medicament group developed apparent rashes. The number of rats in the JAK inhibitor group on which the rashes were remarkably milder than the rashes of the rats in the other dermatologic medicament group was calculated.

Table 5 lists various animal experiment combinations of a JAK inhibitor ointment and currently clinically available dermatological medicaments, and the corresponding experimental results (wherein, the numerical values in the column of relative ameliorating rate=the number of rats in the JAK inhibitor group on which rashes were milder than those of rats in the other dermatologic medicament group/the total number of rats in the JAK inhibitor group x 100%).

TABLE 5
Experimental conditions and results of Examples 5

| | | | | Administration | | | |
|---|---|---|---|---|---|---|---|
| | | | | | Another | | |
| | | | | JAK | dermatologic | Days for | Relative |
| | Type of the | | | inhibitor | medicament | adminis- | Ameliorating |
| EGFR inhibitor | inhibitor | Dosage | Frequency | group | group | tration | Rate |
| Gefitinib | First-generation | 80 | Twice a | 2% | Hydrocortisone | 15 | 80% |
| | EGFR small | mg/kg | day | TD-1473 | ointment | | |
| | molecular | | | ointment | (1%) | | |
| | inhibitor | | | | | | |
| Afatinib | Second-generation | 40 | Once a day | 5% | Hydrocortisone | 14 | 100% |
| | EGFR small | mg/kg | | Tofacitinib | ointment | | |
| | molecular | | | ointment | (1%) | | |
| | inhibitor | | | | | | |
| Osimertinib | Third-generation | 60 | Twice a | 10% | Hydrocortisone | 10 | 66.7% |
| | EGFR small | mg/kg | day | Ruxolitinib | ointment | | |
| | molecular | | | ointment | (1%) | | |
| | inhibitor | | | | | | |
| EAI045 | Fourth-generation | 80 | Once a day | 5% | Hydrocortisone | 11 | 80% |
| | EGFR small | mg/kg | | Itacitinib | ointment | | |
| | molecular | | | ointment | (1%) | | |
| | inhibitor | | | | | | |
| Afatinib | Second-generation | 40 | Once a day | 0.2% | Desonide | 14 | 100% |
| | EGFR small | mg/kg | | Tofacitinib | ointment | | |
| | molecular | | | ointment | (0.05%) | | |
| | inhibitor | | | | | | |
| Afatinib | Second-generation | 40 | Once a day | 0.75% | Clindamycin | 14 | 60% |
| | EGFR small | mg/kg | | Ritlecitinib | ointment (1%) | | |
| | molecular | | | ointment | | | |
| | inhibitor | | | | | | |
| Afatinib | Second-generation | 40 | Once a day | 2% | Fluocinonide | 14 | 75% |
| | EGFR small | mg/kg | | Momelotinib | ointment | | |
| | molecular | | | ointment | (0.05%) | | |
| | inhibitor | | | | | | |
| Afatinib | Second-generation | 40 | Once a day | 2% | Alclometasone | 14 | 75% |
| | EGFR small | mg/kg | | BMS-9 | ointment | | |
| | molecular | | | 86165 | (0.05%) | | |
| | inhibitor | | | ointment | | | |

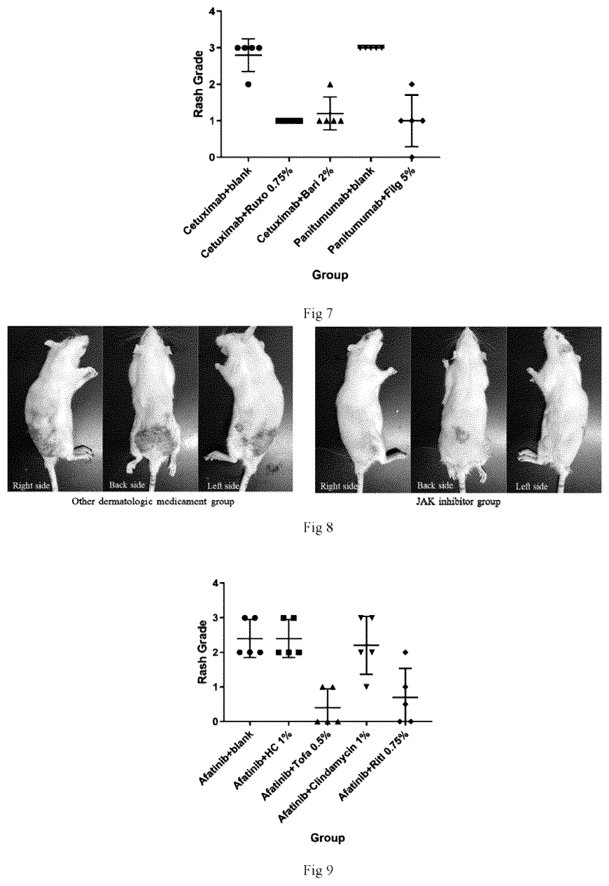

FIG. 8 shows the photographs of left side, back side, and right side of typical rats in another dermatologic medicament group and the JAK inhibitor group in Table 5. FIG. 9 shows the rash grading of the JAK inhibitor group and other dermatologic medicament group at the end of the experiment.

It can be seen from the results in Table 5 that: compared with the currently clinically available dermatological medicaments (almost having no therapeutic effects on rashes caused by EGFR inhibitors), the JAK inhibitor ointment can effectively control rashes caused by EGFR inhibitors.

### Examples 6: Experiment for Demonstrating the Ability of the JAK Inhibitor in Preventing the Diseases or Disorders Caused by Antitumor Agent in Rat Animal Models

Construction of rat models: antitumor agents were administered to 6-week female SD rats by daily gavage, and after several days, the rat developed a large area of rash on the back. There was no difference between the left and right sides of the rash area, and the rash degree was similar on both sides. Like in humans, the rats developed rashes on their faces and bodies after oral administration of antitumor agents. Both have the same cause, and exhibit similar symptoms. Thus, rats are excellent animal models to mimic the rashes induced by the antitumor agents.

After a week of acclimatizing (about 200 g), the SD rats were divided into 10 rats per group. The hair on the back of the rats was gently shaved with an electric shaver on the day before the experiment, and then the intragastric administration was performed. The antitumor agents were dissolved in corresponding solutions and diluted with a PBS buffer solution. The dose was no more than 2 mL per gavage, and the dosages were shown in Table 6. The experiments were divided into a JAK inhibitor group and a control group. After gavage, the back of the rats in the JAK inhibitor group (about 3 cm×3 cm area) was applied with JAK inhibitor ointment (the type and concentration as shown in Table 6); the back of the rats in the control group (about 3 cm×3 cm area) was applied with blank ointment (about 0.5 g). After administration, the rats were fixed by a cylinder for about 4 hrs. Then, the rats were released, wiped with clean water to remove the residual medicament at the application site, and returned to the cage. The gavage frequency of the EGFR inhibitors was shown in Table 6, while the JAK inhibitors and the blank ointment were applied only once a day. The gavage and application tests were repeated every day, until the rats in the control group developed apparent rashes. At this point, the number of rats in the JAK inhibitor group on which the skin kept normal or remarkably milder than the rash in the control group was calculated as the number of rats on which rashes had been effectively inhibited.

Table 6 lists various animal experiment combinations of an antitumor agent and a JAK inhibitor ointment, and the corresponding experimental results (wherein, the numerical values in the column of control rate=the number of rats in the JAK inhibitor group on which rashes were milder than those of rats in the control group/the total number of rats in the JAK inhibitor group x 100%).

TABLE 6
Experimental conditions and results of Example 6

| | | Dosage | | | Concentration | | Control |
|---|---|---|---|---|---|---|---|
| EGFR Inhibitors | Type of the inhibitor | (mg/kg) | Frequency | Administration | wt % | Days | rate |
| Mobocertinib | Small molecule | 15 | Once a | Filgotinib | 1.0% | 10 | 50% |
| | inhibitor of EGFR | | day | ointment | | | |
| Mobocertinib | Small molecule | 15 | Once a | Filgotinib | 3.0% | 10 | 40% |
| | inhibitor of EGFR | | day | ointment | | | |
| Mobocertinib | Small molecule | 15 | Once a | Filgotinib | 5.0% | 10 | 70% |
| | inhibitor of EGFR | | day | ointment | | | |
| Savolitinib | Small molecule | 60 | Once a | Filgotinib | 2.0% | 12 | 50% |
| | inhibitor of EGFR | | day | ointment | | | |
| Savolitinib | Small molecule | 60 | Once a | Filgotinib | 5.0% | 12 | 80% |
| | inhibitor of EGFR | | day | ointment | | | |
| Vandetanib | Small molecule | 50 | Once a | Filgotinib | 0.5% | 12 | 30% |
| | inhibitor of EGFR | | day | ointment | | | |
| Vandetanib | Small molecule | 50 | Once a | Filgotinib | 3.0% | 12 | 50% |
| | inhibitor of EGFR | | day | ointment | | | |
| Lapatinib | Small molecule | 120 | Twice a | Filgotinib | 1.0% | 16 | 60% |
| | inhibitor of EGFR | | day | ointment | | | |
| Lapatinib | Small molecule | 120 | Twice a | Filgotinib | 5.0% | 16 | 90% |
| | inhibitor of EGFR | | day | ointment | | | |
| Almonertinib | Small molecule | 60 | Once a | Peficitinib | 1.0% | 14 | 20% |
| | inhibitor of EGFR | | day | ointment | | | |
| Almonertinib | Small molecule | 60 | Once a | Peficitinib | 5.0% | 14 | 60% |
| | inhibitor of EGFR | | day | ointment | | | |
| Irbinitinib | Small molecule | 80 | Once a | Peficitinib | 0.5% | 16 | 20% |
| | inhibitor of EGFR | | day | ointment | | | |
| Irbinitinib | Small molecule | 80 | Once a | Peficitinib | 2.0% | 16 | 40% |
| | inhibitor of EGFR | | day | ointment | | | |
| BPI-7711 | Small molecule | 60 | Once a | Peficitinib | 3.0% | 12 | 70% |
| | inhibitor of EGFR | | day | ointment | | | |
| Pyrotinib | Small molecule | 100 | Once a | Fedratinib | 1.0% | 14 | 40% |
| | inhibitor of EGFR | | day | ointment | | | |
| Pyrotinib | Small molecule | 100 | Once a | Fedratinib | 2.0% | 14 | 30% |
| | inhibitor of EGFR | | day | ointment | | | |
| Pyrotinib | Small molecule | 100 | Once a | Fedratinib | 5.0% | 14 | 60% |
| | inhibitor of EGFR | | day | ointment | | | |
| Brigatinib | Small molecule | 25 | Once a | Fedratinib | 0.5% | 14 | 20% |
| | inhibitor of EGFR | | day | ointment | | | |
| Brigatinib | Small molecule | 25 | Once a | Fedratinib | 2.0% | 14 | 60% |
| | inhibitor of EGFR | | day | ointment | | | |
| Brigatinib | Small molecule | 25 | Once a | Fedratinib | 5.0% | 14 | 60% |
| | inhibitor of EGFR | | day | ointment | | | |
| Neratinib | Small molecule | 30 | Once a | Baricitinib | 1.0% | 14 | 50% |
| | inhibitor of EGFR | | day | ointment | | | |
| Neratinib | Small molecule | 30 | Once a | Baricitinib | 2.0% | 14 | 50% |
| | inhibitor of EGFR | | day | ointment | | | |
| Olmutinib | Small molecule | 100 | Once a | Baricitinib | 1.0% | 18 | 70% |
| | inhibitor of EGFR | | day | ointment | | | |
| Olmutinib | Small molecule | 100 | Once a | Baricitinib | 3.0% | 18 | 50% |
| | inhibitor of EGFR | | day | ointment | | | |
| Olmutinib | Small molecule | 100 | Once a | Baricitinib | 5.0% | 18 | 80% |
| | inhibitor of EGFR | | day | ointment | | | |
| Bosutinib | Small molecule | 20 | Once a | Tasocitinib | 1.0% | 12 | 30% |
| | inhibitor of EGFR | | day | ointment | | | |
| Bosutinib | Small molecule | 20 | Once a | Tasocitinib | 2.0% | 12 | 50% |
| | inhibitor of EGFR | | day | ointment | | | |
| Neratinib | Small molecule | 30 | Once a | Tasocitinib | 5.0% | 14 | 70% |
| | inhibitor of EGFR | | day | ointment | | | |
| Icotinib | Small molecule | 150 | Twice a | Ruxolitinib | 0.5% | 12 | 30% |
| | inhibitor of EGFR | | day | ointment | | | |
| Icotinib | Small molecule | 150 | Twice a | Ruxolitinib | 2.0% | 12 | 70% |
| | inhibitor of EGFR | | day | ointment | | | |
| Vandetanib | Small molecule | 50 | Once a | Ruxolitinib | 1.0% | 12 | 50% |
| | inhibitor of EGFR | | day | ointment | | | |
| Vandetanib | Small molecule | 50 | Once a | Ruxolitinib | 5.0% | 12 | 100% |
| | inhibitor of EGFR | | day | ointment | | | |
| Lazertinib | Small molecule | 80 | Once a | Ruxolitinib | 3.0% | 16 | 70% |
| | inhibitor of EGFR | | day | ointment | | | |
| Alflutinib | Small molecule | 50 | Once a | Ruxolitinib | 3.0% | 14 | 90% |
| | inhibitor of EGFR | | day | ointment | | | |
| Lapatinib | Small molecule | 120 | Twice a | Abrocitinib | 3.0% | 16 | 50% |
| | inhibitor of EGFR | | day | ointment | | | |
| Lapatinib | Small molecule | 120 | Twice a | Abrocitinib | 5.0% | 16 | 50% |
| | inhibitor of EGFR | | day | ointment | | | |
| Poziotinib | Small molecule | 10 | Once a | Abrocitinib | 0.5% | 14 | 20% |
| | inhibitor of EGFR | | day | ointment | | | |
| Poziotinib | Small molecule | 10 | Once a | Abrocitinib | 3.0% | 14 | 60% |
| | inhibitor of EGFR | | day | ointment | | | |
| Irbinitinib | Small molecule | 80 | Once a | Abrocitinib | 5.0% | 16 | 80% |
| | inhibitor of EGFR | | day | ointment | | | |
| BPI-7711 | Small molecule | 60 | Once a | Deucravacitinib | 1.0% | 12 | 30% |
| | inhibitor of EGFR | | day | ointment | | | |
| BPI-7711 | Small molecule | 60 | Once a | Deucravacitinib | 3.0% | 12 | 50% |
| | inhibitor of EGFR | | day | ointment | | | |
| Dovitinib | Small molecule | 50 | Once a | Deucravacitinib | 3.0% | 14 | 40% |
| | inhibitor of EGFR | | day | ointment | | | |
| Famitinib | Small molecule | 60 | Once a | Deucravacitinib | 5.0% | 16 | 70% |
| | inhibitor of EGFR | | day | ointment | | | |
| Pyrotinib | Small molecule | 100 | Once a | Itacitinib | 3.0% | 14 | 70% |
| | inhibitor of EGFR | | day | ointment | | | |
| Brigatinib | Small molecule | 25 | Once a | Itacitinib | 3.0% | 14 | 30% |
| | inhibitor of EGFR | | day | ointment | | | |
| Almonertinib | Small molecule | 60 | Once a | Itacitinib | 1.0% | 14 | 50% |
| | inhibitor of EGFR | | day | ointment | | | |
| Almonertinib | Small molecule | 60 | Once a | Itacitinib | 5.0% | 14 | 70% |
| | inhibitor of EGFR | | day | ointment | | | |
| Bosutinib | Small molecule | 20 | Once a | Momelotinib | 1.0% | 12 | 30% |
| | inhibitor of EGFR | | day | ointment | | | |
| Bosutinib | Small molecule | 20 | Once a | Momelotinib | 2.0% | 12 | 60% |
| | inhibitor of EGFR | | day | ointment | | | |
| Neratinib | Small molecule | 30 | Once a | Momelotinib | 5.0% | 14 | 70% |
| | inhibitor of EGFR | | day | ointment | | | |
| Famitinib | Small molecule | 60 | Once a | Izencitinib | 1.0% | 16 | 70% |
| | inhibitor of EGFR | | day | ointment | | | |
| Famitinib | Small molecule | 60 | Once a | Izencitinib | 2.0% | 12 | 50% |
| | inhibitor of EGFR | | day | ointment | | | |
| Lapatinib | Small molecule | 120 | Twice a | Izencitinib | 3.0% | 16 | 70% |
| | inhibitor of EGFR | | day | ointment | | | |
| Poziotinib | Small molecule | 10 | Once a | Izencitinib | 3.0% | 14 | 70% |
| | inhibitor of EGFR | | day | ointment | | | |
| Dovitinib | Small molecule | 50 | Once a | Ritlecitinib | 1.0% | 14 | 30% |
| | inhibitor of EGFR | | day | ointment | | | |
| Dovitinib | Small molecule | 50 | Once a | Ritlecitinib | 3.0% | 14 | 30% |
| | inhibitor of EGFR | | day | ointment | | | |
| Bosutinib | Small molecule | 20 | Once a | Ritlecitinib | 2.0% | 6 | 40% |
| | inhibitor of EGFR | | day | ointment | | | |
| Irbinitinib | Small molecule | 80 | Once a | Ritlecitinib | 5.0% | 16 | 40% |
| | inhibitor of EGFR | | day | ointment | | | |
| Zorifertinib | Small molecule | 20 | Once a | upadacitinib | 0.5% | 14 | 30% |
| | inhibitor of EGFR | | day | ointment | | | |
| Zorifertinib | Small molecule | 20 | Once a | upadacitinib | 3.0% | 14 | 70% |
| | inhibitor of EGFR | | day | ointment | | | |
| Mobocertinib | Small molecule | 15 | Once a | upadacitinib | 1.0% | 10 | 70% |
| | inhibitor of EGFR | | day | ointment | | | |
| Mobocertinib | Small molecule | 15 | Once a | upadacitinib | 3.0% | 10 | 70% |
| | inhibitor of EGFR | | day | ointment | | | |
| Savolitinib | Small molecule | 60 | Once a | upadacitinib | 2.0% | 12 | 50% |
| | inhibitor of EGFR | | day | ointment | | | |
| Savolitinib | Small molecule | 60 | Once a | upadacitinib | 5.0% | 12 | 100% |
| | inhibitor of EGFR | | day | ointment | | | |
| Varlitinib | Small molecule | 80 | Twice a | Brepocitinib | 1.0% | 16 | 50% |
| | inhibitor of EGFR | | day | ointment | | | |
| Varlitinib | Small molecule | 80 | Twice a | Brepocitinib | 2.0% | 16 | 50% |
| | inhibitor of EGFR | | day | ointment | | | |
| Bosutinib | Small molecule | 20 | Once a | Brepocitinib | 3.0% | 12 | 70% |
| | inhibitor of EGFR | | day | ointment | | | |
| Bosutinib | Small molecule | 20 | Once a | Brepocitinib | 5.0% | 12 | 90% |
| | inhibitor of EGFR | | day | ointment | | | |
| Neratinib | Small molecule | 30 | Once a | Brepocitinib | 5.0% | 14 | 90% |
| | inhibitor of EGFR | | day | ointment | | | |
| Lazertinib | Small molecule | 80 | Once a | Entrectinib | 0.5% | 16 | 20% |
| | inhibitor of EGFR | | day | ointment | | | |
| Lazertinib | Small molecule | 80 | Once a | Entrectinib | 2.0% | 16 | 60% |
| | inhibitor of EGFR | | day | ointment | | | |
| Alflutinib | Small molecule | 50 | Once a | Entrectinib | 1.0% | 14 | 40% |
| | inhibitor of EGFR | | day | ointment | | | |
| Alflutinib | Small molecule | 50 | Once a | Entrectinib | 5.0% | 14 | 80% |
| | inhibitor of EGFR | | day | ointment | | | |
| Olmutinib | Small molecule | 100 | Once a | Entrectinib | 0.5% | 18 | 30% |
| | inhibitor of EGFR | | day | ointment | | | |
| Olmutinib | Small molecule | 100 | Once a | Entrectinib | 2.0% | 18 | 70% |
| | inhibitor of EGFR | | day | ointment | | | |
| Afatinib | Small molecule | 40 | Once a | Pacritinib | 2.0% | 14 | 40% |
| | inhibitor of EGFR | | day | ointment | | | |
| Afatinib | Small molecule | 40 | Once a | Jaktinib ointment | 2.0% | 14 | 60% |
| | inhibitor of EGFR | | day | | | | |
| Afatinib | Small molecule | 40 | Once a | Ivarmacitinib | 2.0% | 14 | 20% |
| | inhibitor of EGFR | | day | ointment | | | |
| Afatinib | Small molecule | 40 | Once a | Deuruxolitinib | 2.0% | 14 | 90% |
| | inhibitor of EGFR | | day | ointment | | | |
| Afatinib | Small molecule | 40 | Once a | Adelatinib | 2.0% | 14 | 90% |
| | inhibitor of EGFR | | day | ointment | | | |
| Afatinib | Small molecule | 40 | Once a | NDI-034858 | 2.0% | 14 | 40% |
| | inhibitor of EGFR | | day | ointment | | | |
| Dacomitinib | Small molecule | 10 | Once a | Nezulcitinib | 2.0% | 12 | 10% |
| | inhibitor of EGFR | | day | ointment | | | |
| Dacomitinib | Small molecule | 10 | Once a | ATI-1777 | 2.0% | 12 | 90% |
| | inhibitor of EGFR | | day | ointment | | | |
| Dacomitinib | Small molecule | 10 | Once a | TD-8236 | 1.0% | 12 | 100% |
| | inhibitor of EGFR | | day | ointment | | | |
| Dacomitinib | Small molecule | 10 | Once a | INCB-054707 | 2.0% | 12 | 50% |
| | inhibitor of EGFR | | day | ointment | | | |
| Dacomitinib | Small molecule | 10 | Once a | Ropsacitinib | 2.0% | 12 | 30% |
| | inhibitor of EGFR | | day | ointment | | | |
| Dacomitinib | Small molecule | 10 | Once a | AGA-201 | 2.0% | 12 | 70% |
| | inhibitor of EGFR | | day | ointment | | | |
| Dacomitinib | Small molecule | 10 | Once a | ATI-50001 | 2.0% | 12 | 20% |
| | inhibitor of EGFR | | day | ointment | | | |
| Erlotinib | Small molecule | 70 | Once a | Gusacitinib | 2.0% | 15 | 30% |
| | inhibitor of EGFR | | day | ointment | | | |
| Erlotinib | Small molecule | 70 | Once a | Cerdulatinib | 2.0% | 15 | 70% |
| | inhibitor of EGFR | | day | ointment | | | |
| Erlotinib | Small molecule | 70 | Once a | Solcitinib | 2.0% | 15 | 40% |
| | inhibitor of EGFR | | day | ointment | | | |
| Erlotinib | Small molecule | 70 | Once a | Gandotinib | 2.0% | 15 | 90% |
| | inhibitor of EGFR | | day | ointment | | | |
| Erlotinib | Small molecule | 70 | Once a | Roniciclib | 2.0% | 15 | 30% |
| | inhibitor of EGFR | | day | ointment | | | |
| Afatinib | Small molecule | 40 | Once a | R-348 ointment | 5.0% | 14 | 50% |
| | inhibitor of EGFR | | day | | | | |
| Afatinib | Small molecule | 40 | Once a | AT-9283 | 3.0% | 14 | 90% |
| | inhibitor of EGFR | | day | ointment | | | |
| Afatinib | Small molecule | 40 | Once a | FMX-114 | 2.0% | 14 | 10% |
| | inhibitor of EGFR | | day | ointment | | | |
| Afatinib | Small molecule | 40 | Once a | OST-122 | 2.0% | 14 | 30% |
| | inhibitor of EGFR | | day | ointment | | | |
| Afatinib | Small molecule | 40 | Once a | TQ-05105 | 2.0% | 14 | 20% |
| | inhibitor of EGFR | | day | ointment | | | |
| Afatinib | Small molecule | 40 | Once a | ATI-1777 | 2.0% | 14 | 70% |
| | inhibitor of EGFR | | day | ointment | | | |
| Gefitinib | Small molecule | 80 | Twice a | TT-00420 | 2.0% | 15 | 90% |
| | inhibitor of EGFR | | day | ointment | | | |
| Gefitinib | Small molecule | 80 | Twice a | AZD-4205 | 3.0% | 15 | 30% |
| | inhibitor of EGFR | | day | ointment | | | |
| Gefitinib | Small molecule | 80 | Twice a | Repotrectinib | 2.0% | 15 | 70% |
| | inhibitor of EGFR | | day | ointment | | | |
| Gefitinib | Small molecule | 80 | Twice a | INCB-052793 | 2.0% | 15 | 40% |
| | inhibitor of EGFR | | day | ointment | | | |
| Osimertinib | Small molecule | 60 | Twice a | CT-340 ointment | 2.0% | 10 | 90% |
| | inhibitor of EGFR | | day | | | | |
| Osimertinib | Small molecule | 60 | Twice a | BMS-911543 | 2.0% | 10 | 80% |
| | inhibitor of EGFR | | day | ointment | | | |
| Osimertinib | Small molecule | 60 | Twice a | Ilginatinib | 2.0% | 10 | 50% |
| | inhibitor of EGFR | | day | ointment | | | |
| Osimertinib | Small molecule | 60 | Twice a | Zotiraciclib | 2.0% | 10 | 80% |
| | inhibitor of EGFR | | day | ointment | | | |
| Osimertinib | Small molecule | 60 | Twice a | BGB-23339 | 0.5% | 10 | 70% |
| | inhibitor of EGFR | | day | ointment | | | |
| Osimertinib | Small molecule | 60 | Twice a | ICP-332 | 2.0% | 10 | 90% |
| | inhibitor of EGFR | | day | ointment | | | |
| Osimertinib | Small molecule | 60 | Twice a | ESK-001 | 2.0% | 10 | 50% |
| | inhibitor of EGFR | | day | ointment | | | |
| Osimertinib | Small molecule | 60 | Twice a | SYHX-1901 | 2.0% | 10 | 70% |
| | inhibitor of EGFR | | day | ointment | | | |
| Dacomitinib | Small molecule | 10 | Once a | VTX-958 | 2.0% | 12 | 90% |
| | inhibitor of EGFR | | day | ointment | | | |
| Dacomitinib | Small molecule | 10 | Once a | TLL-018 | 2.0% | 12 | 90% |
| | inhibitor of EGFR | | day | ointment | | | |
| Dacomitinib | Small molecule | 10 | Once a | CEE-321 | 3.0% | 12 | 30% |
| | inhibitor of EGFR | | day | ointment | | | |
| Dacomitinib | Small molecule | 10 | Once a | CJ-15314 | 2.0% | 12 | 100% |
| | inhibitor of EGFR | | day | ointment | | | |
| Dacomitinib | Small molecule | 10 | Once a | TD-5202 | 2.0% | 12 | 80% |
| | inhibitor of EGFR | | day | ointment | | | |
| EAI045 | Small molecule | 80 | Once a | ABBV-712 | 2.0% | 11 | 50% |
| | inhibitor of EGFR | | day | ointment | | | |
| EAI046 | Small molecule | 80 | Once a | GLPG-3667 | 2.0% | 11 | 30% |
| | inhibitor of EGFR | | day | ointment | | | |
| EAI047 | Small molecule | 80 | Once a | CPL-116 | 2.0% | 11 | 40% |
| | inhibitor of EGFR | | day | ointment | | | |
| EAI048 | Small molecule | 80 | Once a | AZD-4604 | 2.0% | 11 | 80% |
| | inhibitor of EGFR | | day | ointment | | | |
| EAI049 | Small molecule | 80 | Once a | TAS-8274 | 2.0% | 11 | 70% |
| | inhibitor of EGFR | | day | ointment | | | |
| EAI050 | Small molecule | 80 | Once a | MAX-40279 | 5.0% | 11 | 60% |
| | inhibitor of EGFR | | day | ointment | | | |
| EAI045 | Small molecule | 80 | Once a | TD-3504 | 2.0% | 11 | 40% |
| | inhibitor of EGFR | | day | ointment | | | |
| Erlotinib | Small molecule | 70 | Once a | KN-002 ointment | 2.0% | 15 | 40% |
| | inhibitor of EGFR | | day | | | | |
| Erlotinib | Small molecule | 70 | Once a | AZD-0449 | 2.0% | 15 | 80% |
| | inhibitor of EGFR | | day | ointment | | | |
| Erlotinib | Small molecule | 70 | Once a | R-548 ointment | 2.0% | 15 | 70% |
| | inhibitor of EGFR | | day | | | | |
| Erlotinib | Small molecule | 70 | Once a | AC-410 ointment | 0.5% | 15 | 70% |
| | inhibitor of EGFR | | day | | | | |
| Erlotinib | Small molecule | 70 | Once a | Spebrutinib | 2.0% | 15 | 70% |
| | inhibitor of EGFR | | day | ointment | | | |
| Osimertinib | Small molecule | 60 | Twice a | ONX-0805 | 2.0% | 10 | 30% |
| | inhibitor of EGFR | | day | ointment | | | |
| Osimertinib | Small molecule | 60 | Twice a | AEG-41174 | 2.0% | 10 | 40% |
| | inhibitor of EGFR | | day | ointment | | | |
| Osimertinib | Small molecule | 60 | Twice a | XL-019 ointment | 2.0% | 10 | 50% |
| | inhibitor of EGFR | | day | | | | |
| Osimertinib | Small molecule | 60 | Twice a | CR-4 ointment | 2.0% | 10 | 30% |
| | inhibitor of EGFR | | day | | | | |
| Osimertinib | Small molecule | 60 | Twice a | WP-1066 | 2.0% | 10 | 100% |
| | inhibitor of EGFR | | day | ointment | | | |
| Osimertinib | Small molecule | 60 | Twice a | GDC-0214 | 2.0% | 10 | 80% |
| | inhibitor of EGFR | | day | ointment | | | |
| Osimertinib | Small molecule | 60 | Twice a | ATI-1777 | 2.0% | 10 | 70% |
| | inhibitor of EGFR | | day | ointment | | | |
| Dacomitinib | Small molecule | 10 | Once a | INCB-047986 | 3.0% | 12 | 40% |
| | inhibitor of EGFR | | day | ointment | | | |
| Dacomitinib | Small molecule | 10 | Once a | PF-06263276 | 2.0% | 12 | 60% |
| | inhibitor of EGFR | | day | ointment | | | |
| Dacomitinib | Small molecule | 10 | Once a | R-333 ointment | 2.0% | 12 | 40% |
| | inhibitor of EGFR | | day | | | | |
| Dacomitinib | Small molecule | 10 | Once a | AZD-1480 | 2.0% | 12 | 60% |
| | inhibitor of EGFR | | day | ointment | | | |
| Dacomitinib | Small molecule | 10 | Once a | Tozasertib | 2.0% | 12 | 30% |
| | inhibitor of EGFR | | day | ointment | | | |
| Dacomitinib | Small molecule | 10 | Once a | CS-12192 | 2.0% | 12 | 60% |
| | inhibitor of EGFR | | day | ointment | | | |
| Dacomitinib | Small molecule | 10 | Once a | AC-1101 | 2.0% | 12 | 70% |
| | inhibitor of EGFR | | day | ointment | | | |
| Acalabrutinib | Small molecule | 30 | Once a | Tofacitinib | 2.0% | 14 | 40% |
| | inhibitor of BTK | | day | ointment | | | |
| Acalabrutinib | Small molecule | 30 | Once a | Itacitinib | 0.50% | 14 | 60% |
| | inhibitor of BTK | | day | ointment | | | |
| Acalabrutinib | Small molecule | 30 | Once a | Filgotinib | 5.0% | 14 | 50% |
| | inhibitor of BTK | | day | ointment | | | |
| Dasatinib | Small molecule | 100 | Once a | Tofacitinib | 2.0% | 14 | 40% |
| | inhibitor of BTK | | day | ointment | | | |
| Dasatinib | Small molecule | 100 | Once a | Itacitinib | 0.50% | 14 | 40% |
| | inhibitor of BTK | | day | ointment | | | |
| Dasatinib | Small molecule | 100 | Once a | Filgotinib | 5.0% | 14 | 20% |
| | inhibitor of BTK | | day | ointment | | | |
| Ibrutinib | Small molecule | 30 | Once a | Upadacitinib | 2.0% | 14 | 20% |
| | inhibitor of BTK | | day | ointment | | | |
| Ibrutinib | Small molecule | 30 | Once a | Peficitinib | 1.0% | 14 | 20% |
| | inhibitor of BTK | | day | ointment | | | |
| Ibrutinib | Small molecule | 30 | Once a | TD-1473 | 1.0% | 14 | 60% |
| | inhibitor of BTK | | day | ointment | | | |
| Zanubrutinib | Small molecule | 25 | Twice a | Abrocitinib | 2.0% | 14 | 30% |
| | inhibitor of BTK | | day | ointment | | | |
| Zanubrutinib | Small molecule | 25 | Twice a | Momelotinib | 2.0% | 14 | 70% |
| | inhibitor of BTK | | day | ointment | | | |
| Zanubrutinib | Small molecule | 25 | Twice a | Brepocitinib | 2.0% | 14 | 20% |
| | inhibitor of BTK | | day | ointment | | | |
| GSK-1120212 | Small molecule | 10 | Once a | Brepocitinib | 3.0% | 12 | 90% |
| | inhibitor of MEK | | day | ointment | | | |
| GSK-1120212 | Small molecule | 10 | Once a | Brepocitinib | 5.0% | 12 | 100% |
| | inhibitor of MEK | | day | ointment | | | |
| GSK-1120212 | Small molecule | 10 | Once a | Itacitinib | 2.0% | 12 | 20% |
| | inhibitor of MEK | | day | ointment | | | |
| GSK-1120212 | Small molecule | 10 | Once a | Momelotinib | 2.0% | 12 | 30% |
| | inhibitor of MEK | | day | ointment | | | |
| GSK-1120212 | Small molecule | 10 | Once a | Ruxolitinib | 2.0% | 12 | 100% |
| | inhibitor of MEK | | day | ointment | | | |
| GSK-1120212 | Small molecule | 10 | Once a | Tasocitinib | 2.0% | 12 | 80% |
| | inhibitor of MEK | | day | ointment | | | |
| GSK-1120212 | Small molecule | 10 | Once a | TD-8236 | 2.0% | 12 | 60% |
| | inhibitor of MEK | | day | ointment | | | |
| GSK-1120212 | Small molecule | 10 | Once a | INCB-054707 | 2.0% | 12 | 90% |
| | inhibitor of MEK | | day | ointment | | | |
| GSK-1120212 | Small molecule | 10 | Once a | Ropsacitinib | 2.0% | 12 | 90% |
| | inhibitor of MEK | | day | ointment | | | |
| GSK-1120212 | Small molecule | 10 | Once a | AGA-201 | 2.0% | 15 | 90% |
| | inhibitor of MEK | | day | ointment | | | |
| Selumetinib | Small molecule | 80 | Twice a | Brepocitinib | 0.5% | 15 | 50% |
| | inhibitor of MEK | | day | ointment | | | |
| Selumetinib | Small molecule | 80 | Twice a | Brepocitinib | 2.0% | 15 | 70% |
| | inhibitor of MEK | | day | ointment | | | |
| Selumetinib | Small molecule | 80 | Twice a | Entrectinib | 2.0% | 15 | 90% |
| | inhibitor of MEK | | day | ointment | | | |
| Selumetinib | Small molecule | 80 | Twice a | Izencitinib | 2.0% | 15 | 100% |
| | inhibitor of MEK | | day | ointment | | | |
| Selumetinib | Small molecule | 80 | Twice a | Momelotinib | 2.0% | 15 | 60% |
| | inhibitor of MEK | | day | ointment | | | |
| Selumetinib | Small molecule | 80 | Twice a | Ruxolitinib | 2.0% | 15 | 50% |
| | inhibitor of MEK | | day | ointment | | | |
| Selumetinib | Small molecule | 80 | Twice a | upadacitinib | 2.0% | 15 | 70% |
| | inhibitor of MEK | | day | ointment | | | |
| Selumetinib | Small molecule | 80 | Twice a | ATI-50001 | 2.0% | 15 | 100% |
| | inhibitor of MEK | | day | ointment | | | |
| Selumetinib | Small molecule | 80 | Twice a | Gusacitinib | 2.0% | 15 | 70% |
| | inhibitor of MEK | | day | ointment | | | |
| Selumetinib | Small molecule | 80 | Twice a | Cerdulatinib | 2.0% | 15 | 90% |
| | inhibitor of MEK | | day | ointment | | | |
| Selumetinib | Small molecule | 80 | Twice a | Solcitinib | 2.0% | 15 | 60% |
| | inhibitor of MEK | | day | ointment | | | |
| Trametinib | Small molecule | 20 | iv, 3 times | Deucravacitinib | 1.0% | 15 | 40% |
| | inhibitor of MEK | | a week | ointment | | | |
| Trametinib | Small molecule | 20 | iv, 3 times | Deucravacitinib | 2.0% | 15 | 30% |
| | inhibitor of MEK | | a week | ointment | | | |
| Trametinib | Small molecule | 20 | iv, 3 times | Itacitinib | 2.0% | 15 | 80% |
| | inhibitor of MEK | | a week | ointment | | | |
| Trametinib | Small molecule | 20 | iv, 3 times | Momelotinib | 2.0% | 15 | 90% |
| | inhibitor of MEK | | a week | ointment | | | |
| Trametinib | Small molecule | 20 | iv, 3 times | Ritlecitinib | 2.0% | 15 | 40% |
| | inhibitor of MEK | | a week | ointment | | | |
| Trametinib | Small molecule | 20 | iv, 3 times | Tasocitinib | 2.0% | 15 | 40% |
| | inhibitor of MEK | | a week | ointment | | | |
| Trametinib | Small molecule | 20 | iv, 3 times | Gandotinib | 2.0% | 15 | 60% |
| | inhibitor of MEK | | a week | ointment | | | |
| Trametinib | Small molecule | 20 | iv, 3 times | Roniciclib | 2.0% | 15 | 30% |
| | inhibitor of MEK | | a week | ointment | | | |
| Alpelisib | Small molecule | 65 | Once a | Tofacitinib | 2.0% | 7 | 40% |
| | inhibitor of PI3K | | day | ointment | | | |
| Alpelisib | Small molecule | 65 | Once a | Itacitinib | 0.500% | 7 | 90% |
| | inhibitor of PI3K | | day | ointment | | | |
| Alpelisib | Small molecule | 65 | Once a | Filgotinib | 5.0% | 7 | 100% |
| | inhibitor of PI3K | | day | ointment | | | |
| Alpelisib | Small molecule | 65 | Once a | Izencitinib | 2.0% | 7 | 70% |
| | inhibitor of PI3K | | day | ointment | | | |
| Alpelisib | Small molecule | 65 | Once a | Izencitinib | 5.0% | 7 | 100% |
| | inhibitor of PI3K | | day | ointment | | | |
| Alpelisib | Small molecule | 65 | Once a | ATI-1777 | 5.0% | 7 | 90% |
| | inhibitor of PI3K | | day | ointment | | | |
| Alpelisib | Small molecule | 65 | Once a | OST-122 | 2.0% | 7 | 30% |
| | inhibitor of PI3K | | day | ointment | | | |
| Alpelisib | Small molecule | 65 | Once a | TQ-05105 | 2.0% | 7 | 50% |
| | inhibitor of PI3K | | day | ointment | | | |
| Parsaclisib | Small molecule | 100 | Once a | Baricitinib | 2.0% | 6 | 80% |
| | inhibitor of PI3K | | day | ointment | | | |
| Parsaclisib | Small molecule | 100 | Once a | Fedratinib | 2.0% | 6 | 100% |
| | inhibitor of PI3K | | day | ointment | | | |
| Umbralisib | Small molecule | 130 | Twice a | Tofacitinib | 2.0% | 5 | 100% |
| | inhibitor of PI3K | | day | ointment | | | |
| Umbralisib | Small molecule | 130 | Twice a | Itacitinib | 0.5% | 5 | 20% |
| | inhibitor of PI3K | | day | ointment | | | |
| Ipatasertib | small molecule | 126 | Once a | Tofacitinib | 2% | 13 | 60% |
| | inhibitor of AKT | | day | ointment | | | |
| Ipatasertib | small molecule | 126 | Once a | Itacitinib | 0.50% | 13 | 50% |
| | inhibitor of AKT | | day | ointment | | | |
| Ipatasertib | small molecule | 123 | Once a | Filgotinib | 5.0% | 10 | 50% |
| | inhibitor of AKT | | day | ointment | | | |
| temsirolimus | Small molecule | 50 | iv, Once a | Tofacitinib | 2.0% | 12 | 90% |
| | inhibitor of mORC1, | | day | ointment | | | |
| | mTORC2 | | | | | | |
| temsirolimus | Small molecule | 50 | iv, Once a | Itacitinib | 0.500% | 12 | 50% |
| | inhibitor of mORC1, | | day | ointment | | | |
| | mTORC2 | | | | | | |
| temsirolimus | Small molecule | 50 | iv, Once a | Filgotinib | 5.0% | 12 | 80% |
| | inhibitor of mORC1, | | day | ointment | | | |
| | mTORC2 | | | | | | |
| Apatinib | Small molecule | 90 | Once a | CT-340 ointment | 2.0% | 12 | 70% |
| | inhibitor of VEGFR | | day | | | | |
| Apatinib | Small molecule | 90 | Once a | BMS-911543 | 2.0% | 12 | 80% |
| | inhibitor of VEGFR | | day | ointment | | | |
| Axitinib | Small molecule | 50 | Once a | Zotiraciclib | 2.0% | 16 | 20% |
| | inhibitor of VEGFR | | day | ointment | | | |
| Axitinib | Small molecule | 50 | Once a | BGB-23339 | 0.5% | 16 | 60% |
| | inhibitor of VEGFR | | day | ointment | | | |
| Lenvatinib | Small molecule | 65 | Once a | TLL-018 | 2.0% | 16 | 90% |
| | inhibitor of VEGFR | | day | ointment | | | |
| Lenvatinib | Small molecule | 65 | Once a | CEE-321 | 3.0% | 16 | 60% |
| | inhibitor of VEGFR | | day | ointment | | | |
| Sorafenib | Small molecule | 60 | Twice a | Abrocitinib | 2.0% | 10 | 90% |
| | inhibitor of VEGFR | | day | ointment | | | |
| Sorafenib | Small molecule | 60 | Twice a | Deucravacitinib | 2.0% | 10 | 70% |
| | inhibitor of VEGFR | | day | ointment | | | |
| Dabrafenib + | Small molecule | 100 + 10 | Once a | Baricitinib | 1.0% | 13 | 90% |
| Trametinib | inhibitor of BRAF | | day | ointment | | | |
| Dabrafenib + | Small molecule | 100 + 10 | Once a | upadacitinib | 2.0% | 13 | 80% |
| Trametinib | inhibitor of BRAF | | day | ointment | | | |
| Dabrafenib + | Small molecule | 100 + 10 | Once a | ATI-1777 | 2.0% | 13 | 50% |
| Trametinib | inhibitor of BRAF | | day | ointment | | | |
| Vemurafenib | Small molecule | 110 | Twice a | Baricitinib | 2.0% | 16 | 40% |
| | inhibitor of BRAF | | day | ointment | | | |
| Vemurafenib | Small molecule | 110 | Twice a | Fedratinib | 2.0% | 16 | 80% |
| | inhibitor of BRAF | | day | ointment | | | |
| Vemurafenib | Small molecule | 110 | Twice a | Izencitinib | 2.0% | 16 | 60% |
| | inhibitor of BRAF | | day | ointment | | | |
| Vemurafenib | Small molecule | 110 | Twice a | upadacitinib | 1.0% | 16 | 90% |
| | inhibitor of BRAF | | day | ointment | | | |

It can be seen from the results in Table 6 that: the JAK inhibitor ointment can effectively prevent rashes caused by a small molecular antitumor agent.

### Examples 7: Experiment for Demonstrating the Ability of the JAK Inhibitor in Treating the Diseases or Disorders Caused by Antitumor Agent in Rat Models

After a week of acclimatizing (about 200 g), the SD rats were divided into 10 rats per group. The hair on the back of the rats was gently shaved with an electric shaver on the day before the experiment, and then the intragastric administration was initiated. The antitumor agents were dissolved in corresponding solution and diluted with a PBS buffer solution. The dose was no more than 2 mL per gavage, and the dosages were shown in Table 7. The gavage was performed every day, until the rats developed the symptom of rashes, and at this time the therapeutic experiments were performed. The experiments were divided into a JAK inhibitor group and a control group. During the therapeutic experiment, the rats were continuously subjected to gavage with the antitumor agent every day. After gavage, the back of the rats in the JAK inhibitor group (about 3 cm×3 cm area) was applied with JAK inhibitor ointment; the back of the rats in the control group (about 3 cm×3 cm area) was applied with blank ointment. After administration, the rats were fixed by a cylinder for about 4 hrs. Then, the rats were released, wiped with clean water to remove the residual medicament at the application site, and returned to the cage. The gavage frequency of the antitumor agents was shown in Table 7, while the JAK inhibitors and the blank ointment were administered only once a day. The oral gavage of antitumor agents was repeated every day. The number of rats in the JAK inhibitor group on which the skin returned to normal or remarkably milder than the rashes of the rats in the control group was calculated as the number of rats on which rashes had been effectively treated.

Table 7 lists various animal experiment combinations of an antitumor agent and a JAK inhibitor ointment, and the corresponding experimental results (wherein, the numerical values in the column of control rate=the number of rats in the JAK inhibitor group on which rashes were milder than those of rats in the control group/the total number of rats in the JAK inhibitor group x 100%).

TABLE 7
Experimental conditions and results of Examples 7

| | | | | Modeling | | Concen- | |
|---|---|---|---|---|---|---|---|
| | | | | days for | | tration | |
| | | Dosage | | Adminis- | | wt | Control |
| Inhibitor | Type of the inhibitor | (mg/kg) | Frequency | tration | Administration | % | rate |
| Zanubrutinib | Small molecule | 25 | Twice a day | 10 | Tofacitinib | 0.2% | 10% |
| | inhibitor of BTK | | | | ointment | | |
| Zanubrutinib | Small molecule | 25 | Twice a day | 10 | Tofacitinib | 2.0% | 20% |
| | inhibitor of BTK | | | | ointment | | |
| Zanubrutinib | Small molecule | 25 | Twice a day | 10 | Tofacitinib | 5.0% | 50% |
| | inhibitor of BTK | | | | ointment | | |
| Zanubrutinib | Small molecule | 25 | Twice a day | 10 | Ruxolitinib | 0.2% | 30% |
| | inhibitor of BTK | | | | ointment | | |
| Zanubrutinib | Small molecule | 25 | Twice a day | 10 | Ruxolitinib | 5.0% | 60% |
| | inhibitor of BTK | | | | ointment | | |
| Zanubrutinib | Small molecule | 25 | Twice a day | 10 | Filgotinib | 1.5% | 40% |
| | inhibitor of BTK | | | | ointment | | |
| Zanubrutinib | Small molecule | 25 | Twice a day | 10 | ATI-1777 | 2.0% | 50% |
| | inhibitor of BTK | | | | ointment | | |
| Zanubrutinib | Small molecule | 25 | Twice a day | 10 | Itacitinib | 1.5% | 40% |
| | inhibitor of BTK | | | | ointment | | |
| Acalabrutinib | Small molecule | 30 | Once a day | 10 | Tofacitinib | 2.0% | 30% |
| | inhibitor of BTK | | | | ointment | | |
| Acalabrutinib | Small molecule | 30 | Once a day | 10 | Ruxolitinib | 0.2% | 40% |
| | inhibitor of BTK | | | | ointment | | |
| Acalabrutinib | Small molecule | 30 | Once a day | 10 | Filgotinib | 1.5% | 20% |
| | inhibitor of BTK | | | | ointment | | |
| Acalabrutinib | Small molecule | 30 | Once a day | 10 | Itacitinib | 1.5% | 40% |
| | inhibitor of BTK | | | | ointment | | |
| GSK-1120212 | Small molecule | 10 | Once a day | 10 | Tofacitinib | 2.0% | 40% |
| | inhibitor of MEK | | | | ointment | | |
| GSK-1120212 | Small molecule | 10 | Once a day | 10 | Filgotinib | 1.5% | 30% |
| | inhibitor of MEK | | | | ointment | | |
| Selumetinib | Small molecule | 80 | Twice a day | 10 | Tofacitinib | 0.2% | 30% |
| | inhibitor of MEK | | | | ointment | | |
| Selumetinib | Small molecule | 80 | Twice a day | 10 | Ruxolitinib | 0.2% | 70% |
| | inhibitor of MEK | | | | ointment | | |
| Selumetinib | Small molecule | 80 | Twice a day | 10 | Entrectinib | 2.0% | 40% |
| | inhibitor of MEK | | | | ointment | | |
| Trametinib | Small molecule | 20 | iv, 3 times a | 10 | Itacitinib | 1.5% | 60% |
| | inhibitor of MEK | | week | | ointment | | |
| Trametinib | Small molecule | 20 | iv, 3 times a | 10 | Tofacitinib | 2.0% | 40% |
| | inhibitor of MEK | | week | | ointment | | |
| Alpelisib | Small molecule | 65 | Once a day | 12 | Tofacitinib | 2.0% | 30% |
| | inhibitor of PI3K | | | | ointment | | |
| Alpelisib | Small molecule | 65 | Once a day | 12 | Filgotinib | 1.5% | 70% |
| | inhibitor of PI3K | | | | ointment | | |
| Alpelisib | Small molecule | 65 | Once a day | 12 | Itacitinib | 1.5% | 60% |
| | inhibitor of PI3K | | | | ointment | | |
| Alpelisib | Small molecule | 65 | Once a day | 12 | Izencitinib | 2.0% | 40% |
| | inhibitor of PI3K | | | | ointment | | |
| Apitolisib | Small molecule | 30 | Once a day | 13 | Tofacitinib | 2.0% | 60% |
| | inhibitor of PI3K | | | | ointment | | |
| Ipatasertib | Small molecule | 126 | Once a day | 13 | Tofacitinib | 5.0% | 90% |
| | inhibitor of AKT | | | | ointment | | |
| Ipatasertib | Small molecule | 126 | Once a day | 13 | Filgotinib | 1.5% | 30% |
| | inhibitor of AKT | | | | ointment | | |
| Ipatasertib | Small molecule | 123 | Once a day | 13 | Itacitinib | 5.0% | 70% |
| | inhibitor of AKT | | | | ointment | | |
| temsirolimus | Small molecule | 50 | iv, Once a | 14 | Itacitinib | 5.0% | 80% |
| | inhibitor of mTORC1, | | day | | ointment | | |
| | mTORC2 | | | | | | |
| Ponatinib | Small molecule | 70 | Once a day | 12 | Tofacitinib | 2.0% | 30% |
| | inhibitor of VEGF | | | | ointment | | |
| Ponatinib | Small molecule | 70 | Once a day | 12 | Filgotinib | 1.5% | 20% |
| | inhibitor of VEGF | | | | ointment | | |
| Ponatinib | Small molecule | 70 | Once a day | 12 | Itacitinib | 1.5% | 20% |
| | inhibitor of VEGF | | | | ointment | | |
| Sorafenib | Small molecule | 60 | Twice a day | 12 | Tofacitinib | 2.0% | 50% |
| | inhibitor of VEGF | | | | ointment | | |
| Sorafenib | Small molecule | 60 | Twice a day | 12 | Filgotinib | 1.5% | 30% |
| | inhibitor of VEGF | | | | ointment | | |
| Vandetanib | Small molecule | 40 | Once a day | 10 | Tofacitinib | 2.0% | 60% |
| | inhibitor of EGFR | | | | ointment | | |
| Vandetanib | Small molecule | 40 | Once a day | 10 | Filgotinib | 1.5% | 30% |
| | inhibitor of EGFR | | | | ointment | | |
| Vandetanib | Small molecule | 40 | Once a day | 10 | Itacitinib | 1.50% | 30% |
| | inhibitor of EGFR | | | | ointment | | |
| Dabrafenib + | Small molecule | 100 + 10 | Once a day | 9 | Filgotinib | 1.5% | 20% |
| Trametinib | inhibitor of BRAF | | | | ointment | | |
| Dabrafenib + | Small molecule | 100 + 10 | Once a day | 9 | Itacitinib | 1.50% | 60% |
| Trametinib | inhibitor of BRAF | | | | ointment | | |
| Vemurafenib | Small molecule | 110 | Twice a day | 12 | Filgotinib | 1.5% | 30% |
| | inhibitor of BRAF | | | | ointment | | |
| Vemurafenib | Small molecule | 110 | Twice a day | 12 | Itacitinib | 1.50% | 60% |
| | inhibitor of BRAF | | | | ointment | | |

It can be seen from the results in Table 7 that: the JAK inhibitor ointment can effectively treat rashes caused by an antitumor agent.

### Examples 8: Experiment for Demonstrating the Ability of the JAK Inhibitor in Preventing the Monoclonal Antibody Inhibitors-Induced Rashes in Rat Models

After a week of acclimatizing (about 200 g), the SD rats were divided into 10 rats per group. The hair on the back of the rats was gently shaved with an electric shaver on the day before the experiment, and then the administration was performed. The experiments were divided into a JAK inhibitor group and a control group. A monoclonal antibody solution diluted with normal saline was injected twice a week into the tail vein of rats, for which the injection rate and time were shown in Table 8. After injection, the back of the rats in the JAK inhibitor group (about 3 cm×3 cm area) was applied with JAK inhibitor ointment every day; the back of the rats in the control group (about 3 cm×3 cm area) was applied with blank ointment (about 0.5 g). After applying the medicament, the rats were fixed by a cylinder for about 4 hrs. Then, the rats were released, wiped with clean water to remove the residual medicament at the application site, and returned to the cage. The rats were subjected to tail vein injection twice a week, and were applied with the ointment once a day, until the rats in the control group developed apparent rashes. The number of rats in the JAK inhibitor group on which the skin kept normal or remarkably milder than the rashes of the rats in the control group 10-14 days after the application was recorded and calculated as the number of rats on which rashes had been effectively inhibited.

Table 8 lists various animal experiment combinations of a monoclonal antibody inhibitor and a JAK inhibitor ointment, and the corresponding experimental results (wherein, the numerical values in the column of control rate=the number of rats in the JAK inhibitor group on which rashes were milder than those of rats in the control group/the total number of rats in the JAK inhibitor group x 100%).

TABLE 8
Experimental conditions and results of Examples 8

| Monoclonal | | | | | Con- | | |
|---|---|---|---|---|---|---|---|
| Antibody | Type of the | | | Adminis- | cen- | | Control |
| Inhibitor | inhibitor | Dosage | Frequency | tration | tration | Days | rate |
| Cetuximab | Monoclonal | 100 mg/kg, 15 min | Tail vein | Baricitinib | 1.0% | 10 | 40% |
| | antibody | with an injection rate | injection twice a | ointment | | | |
| | | of 1.3 ml/kg/min for | week | | | | |
| | | each time | | | | | |
| Cetuximab | Monoclonal | 100 mg/kg, 15 min | Tail vein | Abrocitinib | 3.0% | 10 | 70% |
| | antibody | with an injection rate | injection twice a | ointment | | | |
| | | of 1.3 ml/kg/min for | week | | | | |
| | | each time | | | | | |
| Panitumumab | Monoclonal | 10 mg/kg | Tail vein | Itacitinib | 1.0% | 14 | 60% |
| | antibody | | injection twice a | ointment | | | |
| | | | week | | | | |
| Rituximab | CD20 | 60 mg/kg | Tail vein | Tofacitinib | 2% | 14 | 40% |
| | | | injection twice a | ointment | | | |
| | | | week | | | | |

It can be seen from the results in Table 8 that: the JAK inhibitor ointment can effectively prevent rashes caused by monoclonal antibody inhibitors.

### Example 9: Clinical Effects of JAK Inhibitors on Antitumor Agents-Induced Rash

The subjects tested were from patients who receiving targeted therapy and/or immunotherapy and developing rashes. The patient receiving targeted therapy is undergoing treatment with cetuximab, or other antibody antitumor agents; the patient receiving immunotherapy is undergoing CTLA-4 inhibitor (for example: Ipilimumab) and/or PD-1/PD-L1 inhibitors (eg: Pembrolizumab, Nivolumab, etc.) treatment. Those who meet the diagnostic criteria of rash, NCI-CTCAE v5.0 assessment grade 1 or above, and symptoms last for more than 1 week.

The rash diagnostic criteria refer to NCI-CTCAE v5.0 and ASCO guidelines, and the rash caused by targeted therapy and immunotherapy are classified as separate categories.

The experiment was divided into treatment group and control group. During the process of receiving targeted therapy and immunotherapy, the treatment group: the rash area was washed with clean water, and JAK ointment was applied to the affected area 3 times a day in the morning, noon and evening; In the control group, the rash area was cleaned with clean water, and blank ointment was applied to the affected area 3 times a day in the morning, noon and evening. The course of treatment was 4 weeks. Patients were followed up by telephone every week, clinical assessment form was completed: The assessment form consisted of 9 items: previous treatment, treatment with JAK or blank ointment, home treatment, adjuvant treatment, wound type, lesion assessment (width and length in centimeters), the skin around the lesion, the quality-of-life assessment, and whether to suspend medication assessment. Skin biopsy was performed when necessary and evaluated by an expert pathologist.

The number, area size and trend of rash lesions in the treated and non-treated sites were assessed weekly to evaluate the efficacy. The efficacy was evaluated as follows:
Clinical control: symptoms disappeared at the end of treatment;
Significantly effective: at the end of treatment, the symptom was reduced by 2 grades;
Effective: at the end of treatment, the symptom was reduced by 1 grade;
Ineffective: Patients who did not meet the above criteria.

The rash response rate (clinical control+significantly effective+effective)/the total number of cases in the group*100% was calculated by the above evaluation method.

Table 9 lists the different combinations of antitumor agents and ointments, the relative response rate=(clinical control+significantly effective+effective)/the total number of cases in this group*100%.

TABLE 9
Experimental conditions and results of Examples 9

| | | | | Relative |
|---|---|---|---|---|
| Antitumor | | | | Response |
| Agent | Target | Administration | Frequency | Rate |
| Panitumumab | EGFR | 3.2% Tofacitinib | 3 times a day | 40% |
| | | ointment | | |
| | | Blank ointment | 3 times a day | 10% |
| Ipilimumab | CTLA-4 | 3.2% Tofacitinib | 3 times a day | 50% |
| | | ointment | | |
| | | Blank ointment | 3 times a day | 10% |
| Pembrolizumab | PD-1 | 3.2% Tofacitinib | 3 times a day | 40% |
| | | ointment | | |
| | | Blank ointment | 3 times a day | 10% |
| Ipilimumab + | PD-1 + | 3.2% Tofacitinib | 3 times a day | 50% |
| Nivolumab | CTLA-4 | ointment | | |
| | | Blank ointment | 3 times a day | 10% |

The results in Table 9 showed that JAK ointment (tofacitinib ointment) have a certain relieving effect on rash in patients who treated with targeted therapies (Cetuximab, Panitumumab) and immunotherapies (CTLA-4 inhibitors, and/or PD-1/PD-L1 inhibitors).

1-161. (canceled)

162. A method of preventing or treating a cutaneous disease or disorder associated with an antitumor agent, the method comprising:
administering to a subject in need thereof a JAK inhibitor.

163. The method of claim 162, wherein the JAK inhibitor comprises ruxolitinib, tofacitinib, oclacitinib, fedratinib, peficitinib, upadacitinib, barictinib, fligotinib, decemotinib, cerdulatinib, lestaurtinib, pacritinib, momelotinib, gandotinib, abrocitinib, solcitinib, SHR-0203, itacitinib, PF-06651600, BMS-986165, Cucurbitacin I, CHZ868, TD-1473, zotiraciclib, alkotinib, jaktinib, AZD-4205, DTRMHS-07, KL130008, WXSH-0150, TQ05105, WXFL10203614, GLPG0634, CEP-33779, R-348, itacitinib, ritlecitinib, brepocitinib, tasocitinib, deucravacitinib, INCB-039110, izencitinib, entrectinib, ivarmacitinib, deuruxolitinib, adelatinib, NDI-034858, nezulcitinib, ATI-01777, TD-8236, INCB-054707, ropsacitinib, AGA-201, ATI50001, gusacitinib, cerdulatinib, roniciclib, AT-9283, FMX-114, OST-122, TT-00420, repotrectinib, INCB-052793, CT-340, BMS-911543, ilginatinib, BGB-23339, ICP-332, ESK-001, SYHX-1901, VTX-958, TLL-018, CEE-321, CJ-15314, TD-5202, ABBV-712, GLPG-3667, CPL-116, AZD-4604, TAS-8274, MAX-40279, TD-3504, KN-002, AZD-0449, R-548, AC-410, spebrutinib, ONX-0805, AEG-41174, XL-019, CR-4, WP-1066, GDC-0214, INCB-047986, PF-06263276, R-333, AZD-1480, tozasertib, CS-12192 and/or AC-1101.

164. The method of claim 162, wherein the JAK inhibitor comprises a compound of formula (I) or a pharmaceutically acceptable salt thereof: 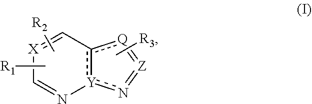
wherein
Q, X, Y, and Z are independently N or C, and
R₁, R₂ and R₃ are independently a 5-6-membered aromatic ring, 5-6-membered heteroaromatic ring, 5-6-membered cycloalkyl, 5-6-membered heterocycloalkyl, amino, or amido, the aromatic ring, heteroaromatic ring, cycloalkyl, and/or heterocycloalkyl being optionally substituted by a substituent.

165. The method of claim 164, wherein the JAK inhibitor comprises:

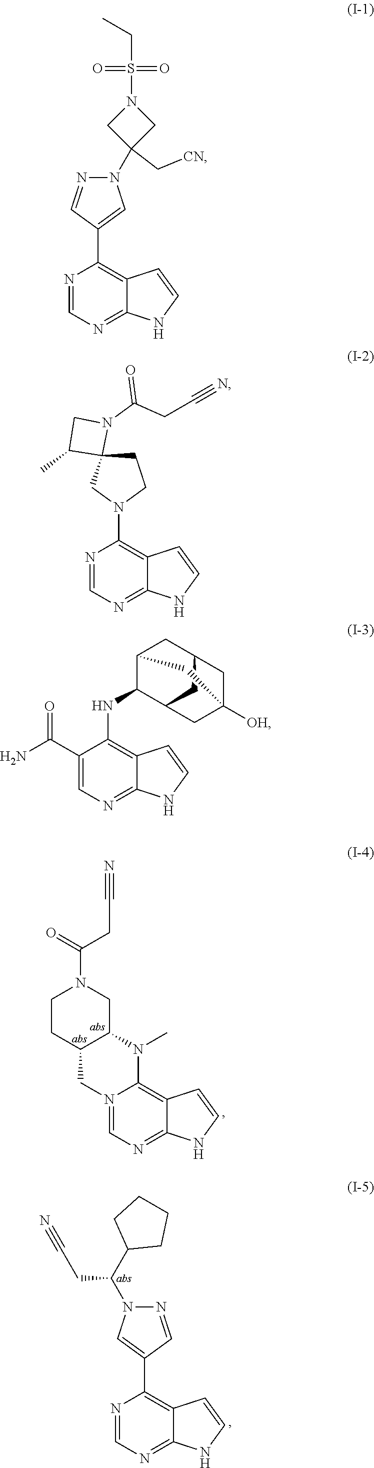

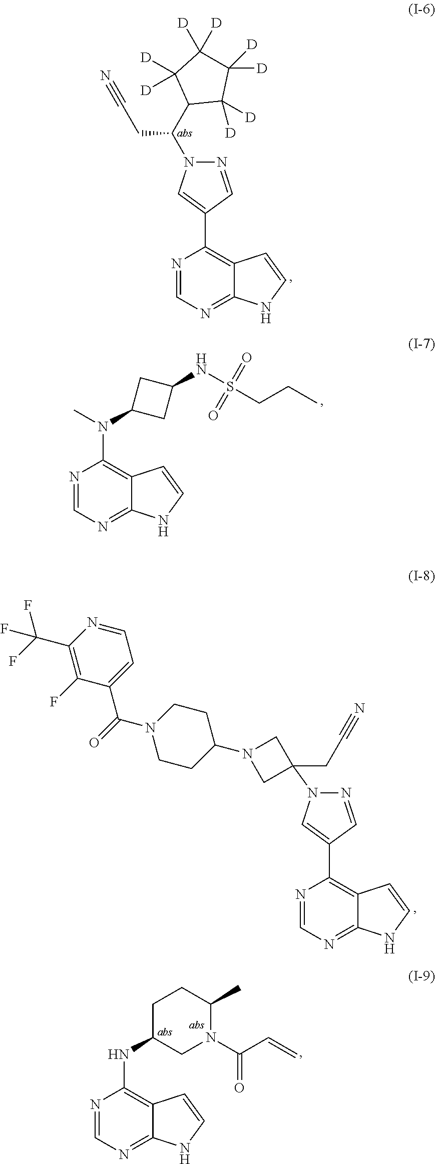 [SMILES: [2H]C1([2H])C([C@@H](CC#N)n2cc(-c3ncnc4[nH]ccc34)cn2)C([2H])([2H])C([2H])([2H])C1([2H])[2H] ; CCCS(=O)(=O)N[C@H]1C[C@@H](N(C)c2ncnc3[nH]ccc23)C1 ; N#CCC1(n2cc(-c3ncnc4[nH]ccc34)cn2)CN(C2CCN(C(=O)c3ccnc(C(F)(F)F)c3F)CC2)C1 ; C=CC(=O)N1C[C@@H](Nc2ncnc3[nH]ccc23)CC[C@H]1C]

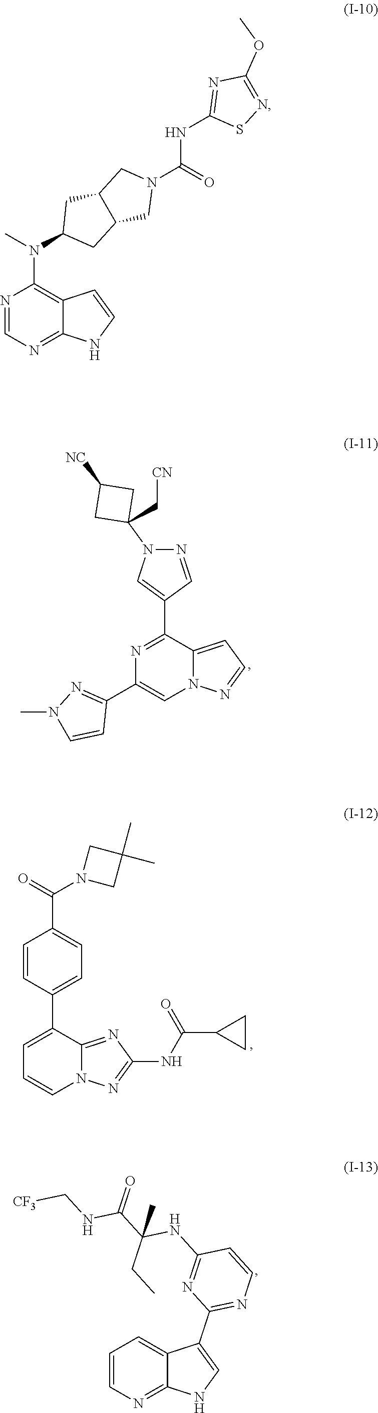 [SMILES: COc1nsc(NC(=O)N2C[C@H]3C[C@H](N(C)c4ncnc5[nH]ccc45)C[C@H]3C2)n1 ; Cn1ccc(-c2cn3nccc3c(-c3cnn([C@]4(CC#N)C[C@@H](C#N)C4)c3)n2)n1 ; CC1(C)CN(C(=O)c2ccc(-c3cccn4nc(NC(=O)C5CC5)nc34)cc2)C1 ; CC[C@@](C)(Nc1ccnc(-c2c[nH]c3ncccc23)n1)C(=O)NCC(F)(F)F]

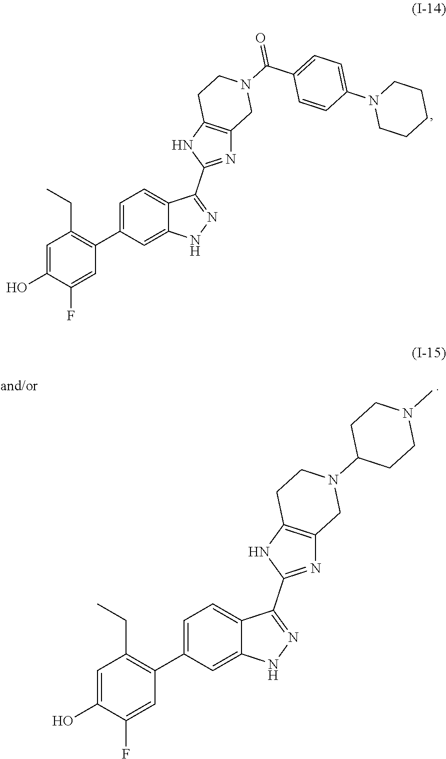 [SMILES: CCc1cc(O)c(F)cc1-c1ccc2c(-c3nc4c([nH]3)CCN(C(=O)c3ccc(N5CCCCC5)cc3)C4)n[nH]c2c1 ; CCc1cc(O)c(F)cc1-c1ccc2c(-c3nc4c([nH]3)CCN(C3CCN(C)CC3)C4)n[nH]c2c1]

166. The method of claim 162, wherein the JAK inhibitor comprises a compound of formula (II): 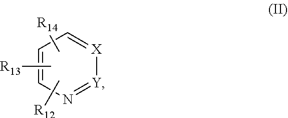
wherein
X and Y are independently C or N, and
R₁₂, R₁₃, and R₁₄ independently comprise H, protium deuterium, tritium, C₁-C₅ alkyl, halogen, alkoxy, amino, amido, sulfonamido, linear alkyl, cycloalkyl, heterocyloalkyl, alkenyl, alkynyl, aryl, or heteroaryl.

167. The method of claim 166, wherein the JAK inhibitor comprises:

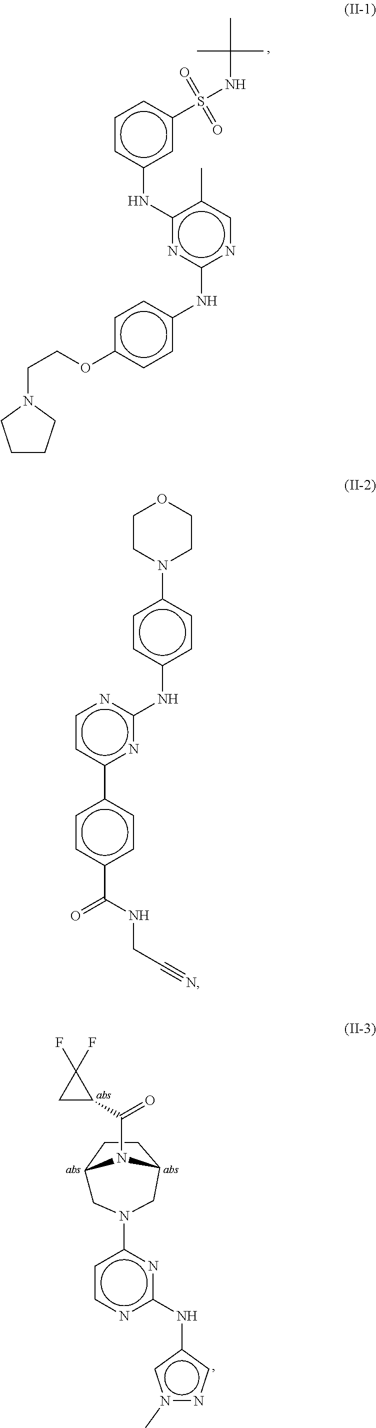

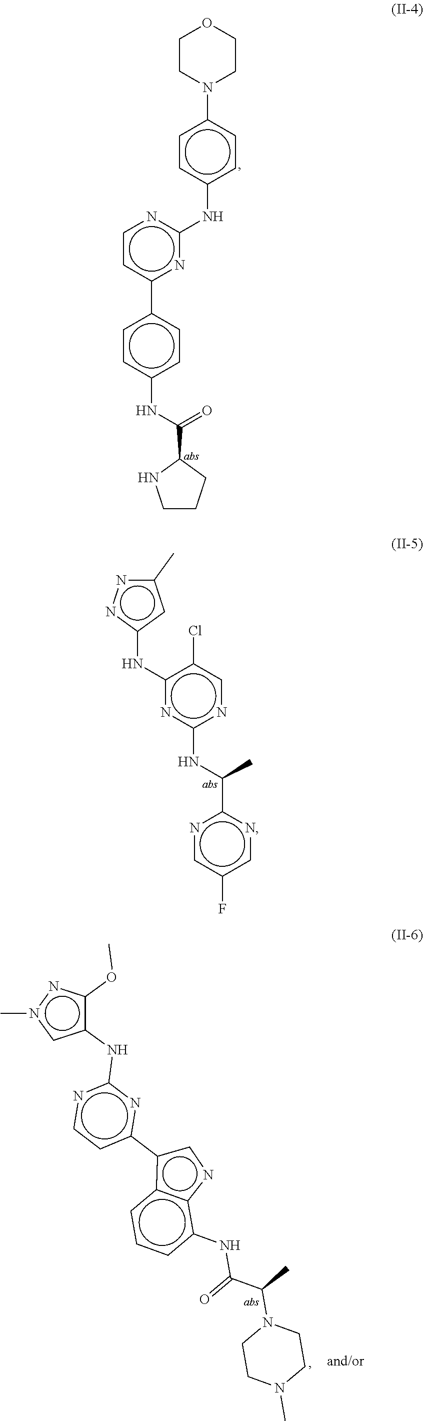

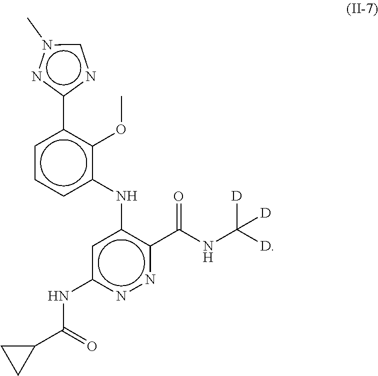

168. The method of claim 162, wherein the JAK inhibitor comprises a compound of formula (III): 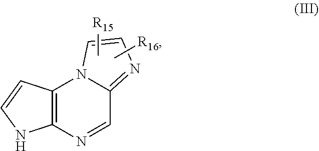
wherein
R₁₅ and R₁₆ are independently H, cycloalkyl, heterocycloalkyl, aryl, or heteroaryl, the cycloalkyl, heterocycloalkyl, aryl, and/or heteroaryl being optionally substituted by a substituent, the substituent being amido, alkyl, cycloalkyl, heterocycloalkyl, cyanogroup, amino, hydroxyl, or halogen.

169. The method of claim 168, wherein the JAK inhibitor comprises

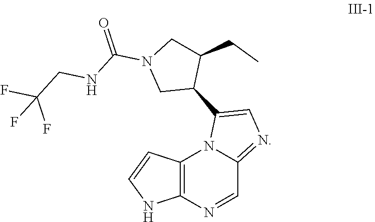 [SMILES: CC[C@@H]1CN(C(=O)NCC(F)(F)F)C[C@@H]1c1cnc2cnc3[nH]ccc3n12]

170. The method of claim 162, wherein the JAK inhibitor comprises a structure of formula (IV): 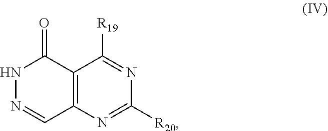
wherein
R₁₉ and R₂₀ are independently H, nitro, 4-10-membered cycloalkyl, 4-10-membered heterocycloalkyl, 4-10-membered aryl, or 4-10-membered heteroaryl, the intro, cycloakyl, heterocycloalkyl, aryl, heteroaryl optionally being substituted by cyanogroup, alkyl, cycloalkyl, heterocycloalkyl, or hydroxyl.

171. The method of claim 170, wherein the JAK inhibitor comprises

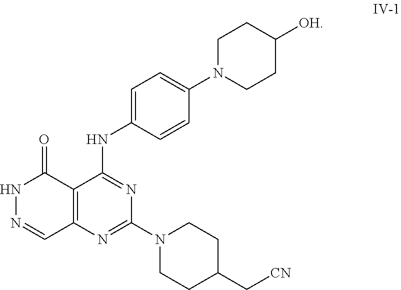 [SMILES: N#CCC1CCN(c2nc(Nc3ccc(N4CCC(O)CC4)cc3)c3c(=O)[nH]ncc3n2)CC1]

172. The method of claim 162, wherein the antitumor agent comprises a targeted therapeutic agent and/or an immunotherapeutic agent.

173. The method of claim 172, wherein the targeted therapeutic agent is an EGFR inhibitor, MEK inhibitor, ALK inhibitor, BTK inhibitor, PI3K inhibitor, AKT inhibitor, VEGFR inhibitor, mTOR inhibitor, HDAC inhibitor, KIT inhibitor, FGFR inhibitor, FAK inhibitor, BCRP inhibitor, EGFRicNIET inhibitor, SRC inhibitor, or combination thereof.

174. The method of claim 172, wherein the immunotherapeutic agent is an immune checkpoint inhibitor, a modified immune cell, and/or a vaccine.

175. The method of claim 172, wherein the immunotherapeutic agent is PD-1 inhibitor, a PD-L1 inhibitor, and/or a CTLA-4 inhibitor.

176. The method of claim 162, wherein the cutaneous disease or disorder comprises rash.

177. The method of claim 162, wherein the cutaneous disease or disorder comprises an EGFR inhibitor administration-associated rash.

178. The method of claim 177, wherein the EGFR inhibitor comprises cetuximab, gefitinib, erlotinib, icotinib, sapitinib, afatinib, lapatinib, vandetanib, neratinib, brigatinib, panitumumab, necitumumab, nimotuzumab, tesevatinib, allitinib, theliatinib, rociletinib, canertinib, AZD3759, YZJ-0318, neptinib, naquotinib, PF-06747775, SPH1188-11, poziotinib, epitinib, varlitinib, alflutinib, HM61713, CK-101, pyrotinib, larotinib, HS-10296, AP32788, simotinib, GMA204, virlitinib, yinlitinib, nazartinib, olmutinib, osimertinib, dacomitinib, abivertinib, EA1045, lazertinib, mobocertinib, savolitinib, almonertinib, trastuzumab, tepotinib, irbinitinib, cemiplimab, mereletinib, bosutinib, befotertinib, poziotinib, BPI-7711, SKLB-1028, famitinib, dovitinib, and/or zorifertinib.

179. The method of claim 162, wherein the subject is a cancer patient.

180. The method of claim 162, wherein the JAK inhibitor is prepared for transdermal administration.

181. The method of claim 162, wherein a concentration of the JAK inhibitor in the medicament is in a range of from 0.01 to 10 wt. %.
